(19) United States
(12) Patent Application Publication
(10) Pub. No.: US 2026/0335341 A1
(43) Pub. Date: Oct. 29, 2026

(54) METHODS FOR TREATING PAEDIATRIC NEUROLOGICAL DISORDERS

(71) Applicant: Neurotech International Limited, Melbourne, Victoria (AU)

(72) Inventors: Allan William Cripps, Hope Island, Queensland (AU); Esra Isikgel, Wheelers Hill, Victoria (AU); Alexandra Elizabeth Marion Andrews, Nedlands, Western Australia (AU); Thomas George Duthy, Beaumont, South Australia (AU)

(21) Appl. No.: 19/113,385

(22) PCT Filed: Oct. 5, 2023
(86) PCT No.: PCT/AU2023/050969

§ 371 Date: Mar. 19, 2025

(30) Foreign Application Priority Data
Oct. 6, 2022 (AU) 2022902914

(51) Int. Cl. A61K 31/00; A61K 31/196; A61K 31/573; A61K 31/635; A61K 31/675; A61P 25/00; A61P 25/28; A61P 29/00

(52) U.S. Cl. CPC A61K 31/658; A61K 31/196; A61K 31/573; A61K 31/635; A61K 31/675; A61P 25/00; A61P 25/28; A61P 29/00

(57) ABSTRACT

The present invention relates to compositions comprising cannabinoids. The present invention also relates to pharmaceutical compositions, dosage forms and methods of treating paediatric neurological disorders by administering the composition to a patient in need thereof.

### FIELD OF THE INVENTION

The present invention relates to compositions comprising cannabinoids. The present invention also relates to pharmaceutical compositions, dosage forms and methods of treating paediatric neurological disorders by administering the composition to a patient in need thereof.

### BACKGROUND

The following discussion of the background art is intended to facilitate an understanding of the present invention only. The discussion is not an acknowledgement or admission that any of the material referred to is or was part of the common general knowledge as at the priority date of the application.

### A. Neuroinflammation

Neuroinflammation refers to the process whereby the brain's innate immune system is triggered following an inflammatory challenge such as those posed by injury, infection, exposure to a toxin, neurodegenerative disease, or aging. Neuroinflammation is implicated in contributing to a variety of neurologic and somatic illnesses including Alzheimer's disease (AD), Parkinson's disease (PD), multiple sclerosis, amyotrophic lateral sclerosis, cerebral ischemia, traumatic brain injury, rheumatoid arthritis, chronic migraine, epilepsy, autism spectrum disorder (ASD), attention deficit hyperactivity disorder (ADHD), cerebral palsy and relevant subtypes, neuropathic pain, and depression.

In the central nervous system (CNS), the innate immune response plays a significant role in both physiological and pathological conditions. CNS diseases including traumatic brain injury, ischemic stroke, brain tumor, and cerebrovascular and neurodegenerative diseases trigger a cascade of events broadly defined as neuroinflammation, which is characterized by the activation of the microglia and astrocyte population. On the other hand, microglial and astrocyte activation, T lymphocyte infiltration, and overproduction of inflammatory cytokines have been demonstrated in association with neuronal alteration in both animal and human tissues. Neuroinflammation is therefore an important topic in contemporary neuroscience.

Inflammatory cytokines/markers or proinflammatory cytokines/markers are types of signaling molecules that are secreted from immune cells like helper T cells and macrophages and certain other cell types that promote the process of neuro-inflammation and general inflammatory processes. These include interleukin-1 (IL-1), IL-12, and IL-18, tumor necrosis factor alpha (TNF-α), interferon gamma (IFNγ) and granulocyte-macrophage colony stimulating factor (GM-CSF). These inflammatory cytokines are predominantly produced by and involved in the upregulation of inflammatory reactions and play an important role in mediating the innate immune response.

### B. Neurological Disorders and Paediatric Neurological Disorders

Examples of neurological disorders that are “neuro-inflammatory based” include: Alzheimer's disease (Alzheimer's disease is the most prevalent chronic, progressive neurodegenerative disease, and cause of dementia); Parkinson's disease; multiple sclerosis; amyotrophic lateral sclerosis; cerebral ischemia; traumatic brain injury; rheumatoid arthritis; chronic migraine; epilepsy; autism spectrum disorder; attention deficit hyperactivity disorder (ADHD); cerebral palsy and relevant subtypes; neuropathic pain; and depression.

Examples of paediatric neurological disorders that are “neuro-inflammatory based” include: Paediatric Autoimmune Neuropsychiatric Syndrome (PANS), and subsets of PANS, including Paediatric Autoimmune Neuropsychiatric Disorder Associated with Streptococcal Infections (PANDAS) and Paediatric Infection Triggered Neuropsychiatric Disorder (PITAND). Children with PANS often present with neuropsychiatric symptoms and disorders, including obsessive-compulsive disorder (OCD), Tourette syndrome and tics (involuntary, purposeless movements).

OCD and Tourette syndrome symptoms in these cases of PANS and PANDAS have been hypothesized to arise from the development of autoantibodies that cross-react with proteins normally expressed in the brain; this mechanism is known as molecular mimicry. The association of immune dysregulation with OCD and Tourette syndrome symptoms in this subset of paediatric patients is increasingly clear.

A TSPO/PK PET imaging study of microglial activation examined both noninfectious Tourette syndrome and PANDAS (Kurlan R. Tourette's syndrome. New England Journal of Medicine. 2010; 363 (24): 2332-2338; Leckman J. F. Tourette's syndrome. The Lancet. 2002; 360 (9345): 1577-1586. doi: 10.1016/s0140-6736 (02) 11526-1). Children with PANDAS had increased PK binding in the striatum with respect to adult healthy controls; this corresponds with increased striatal volumes previously described during acute illness in PANDAS patients (Leckman J. F. Tourette's syndrome. The Lancet. 2002; 360 (9345): 1577-1586. doi: 10.1016/s0140-6736 (02) 11526-1; Williams K., Bloch M. H., State M. W., Pittenger C. Tourette syndrome. In: Charney D. S., editor. Neurobiology of Mental Illness. 4th. New York, NY, USA: Oxford University Press; 2013).

Considerable evidence suggests that immune dysregulation may contribute to the pathophysiology Tourette syndrome and PANS/PANDAS (Leckman J. F. Tourette's syndrome. The Lancet. 2002; 360 (9345): 1577-1586. doi: 10.1016/s0140-6736 (02) 11526-1; Williams K., Bloch M. H., State M. W., Pittenger C. Tourette syndrome. In: Charney D. S., editor. Neurobiology of Mental Illness. 4th. New York, NY, USA: Oxford University Press; 2013; Swedo S. E. Pediatric autoimmune neuropsychiatric disorders associated with streptococcal infections (PANDAS) Molecular Psychiatry. 2002; 7 (supplement 2): S24-S25). However, two recent studies, using different methodologies, have suggested abnormalities in microglial activation in patients with Tourette syndrome and PANS/PANDAS. Both studies focused on the basal ganglia.

Recent studies have used in vivo positron emission tomography (PET) imaging with (¹¹)C-[R]-PK11195 (PK), a ligand that binds to the transporter protein (TSPO), which is expressed by activated microglia. Increased PK binding, indicative of inflammatory microglial activation, was observed in the caudate nuclei bilaterally in children with Tourette syndrome and PANS/PANDAS (Williams K. A., Swedo S. E. Post-infectious autoimmune disorders: sydenham's chorea, PANDAS and beyond. Brain Research. 2015; 1617:144-154; Frick L. R., Williams K., Pittenger C. Microglial dysregulation in psychiatric disease. Clinical and Developmental Immunology. 2013; 2013:10; Ji K., Miyauchi J., Tsirka S. E. Microglia: an active player in the regulation of synaptic activity. Neural Plasticity. 2013; 2013:9).

While evidence for microglial dysregulation in OCD, Tourette syndrome, and PANS/PANDAS is growing, the case for microglial dysregulation is strongest in the case of for these neuro-disorders; recent postmortem and PET imaging studies have produced convergent evidence for increased microglial activation in the striatum in patients with complex neuro-inflammatory disorders (Paolicelli R. C., Bolasco G., Pagani F., et al. Synaptic pruning microglia for by is necessary normal brain development. Science. 2011; 333 (6048): 1456-1458; Schafer D. P., Lehrman E. K., Kautzman A. G., et al. Microglia sculpt postnatal neural circuits in an activity and complement-dependent manner. Neuron. 2012; 74 (4): 691-705). This is complemented by studies in the Hdc-KO mouse model of Tourette syndrome.

### C. Microglia Activation|Neurodegeneration

Microglia cells are the unique residential macrophages of the central nervous system (CNS). They play an important role during CNS development and adult homeostasis. They have a major contribution to adult neurogenesis and neuroinflammation (Zhan Y., Paolicelli R. C., Sforazzini F., et al. Deficient neuron-microglia signaling results in impaired functional brain connectivity and social behavior. Nature Neuroscience. 2014; 17 (3): 400-406; Guruswamy R, EIAli A. Complex Roles of Microglial Cells in Ischemic Stroke Pathobiology: New Insights and Future Directions. Int J Mol Sci. 2017; 18:18). Thus, they participate in the pathogenesis of neurodegenerative diseases and contribute to aging. They play an important role in sustaining and breaking the blood-brain barrier. As innate immune cells, they contribute substantially to the immune response against infectious agents affecting the CNS (Xiong X Y, Liu L, Yang Q W. Functions and mechanisms of microglia/macrophages in neuroinflammation and neurogenesis after stroke. Prog Neurobiol. 2016; 142:23-44). They also play a major role in the growth of tumours of the CNS. Microglia are consequently the key cell population linking the nervous and the immune system (Xiong X Y, Liu L, Yang Q W. Functions and mechanisms of microglia/macrophages in neuroinflammation and neurogenesis after stroke. Prog Neurobiol. 2016; 142:23-44).

Under physiological conditions, ramified, resting microglia provides a neuroprotective environment (David S, Greenhalgh A D, Kroner A. Macrophage and microglial plasticity in the injured spinal cord. Neuroscience. 2015; 307:311-18; Bieber K, Autenrieth S E. Insights how monocytes and dendritic cells contribute and regulate immune defense against microbial pathogens. Immunobiology. 2015; 220:215-26). However, most CNS pathologies, as well as regenerative efforts, include activation of microglia with corresponding inflammatory events (Hoogland I C, Houbolt C, van Westerloo D J, van Gool W A, van de Beek D. Systemic inflammation and microglial activation: systematic review of animal experiments. J Neuroinflammation. 2015; 12:114; Ascoli B M, Géa LP, Colombo R, Barbé-Tuana F M, Kapczinski F, Rosa A R. The role of macrophage polarization on bipolar disorder: identifying new therapeutic targets. Aust N Z J Psychiatry. 2016; 50:618-30; Cherry J D, Olschowka J A, O'Banion M K. Neuroinflammation and M2 microglia: the good, the bad, and the inflamed. J Neuroinflammation. 2014; 11:98). Activated, inflammatory microglia are thus neurotoxic and kill neurons by engulfing them or releasing various neurotoxic molecules and factors, including reactive oxygen species (ROS), glutamate, Fas-ligand, tumour necrosis factor α (TNFα) and others (Loane D J, Kumar A. Microglia in the TBI brain: the good, the bad, and the dysregulated. Exp Neurol. 2016; 275:316-27; Nakagawa Y, Chiba K. Diversity and plasticity of microglial cells in psychiatric and neurological disorders. Pharmacol Ther. 2015; 154:21-35).

Activated microglia driving chronic neuroinflammation have also been shown to substantially contribute to aging of the CNS (Loane D J, Kumar A. Microglia in the TBI brain: the good, the bad, and the dysregulated. Exp Neurol. 2016; 275:316-27), chronic neuropathic pain (Orihuela R, McPherson C A, Harry G J. Microglial M1/M2 polarization and metabolic states. Br J Pharmacol. 2016; 173:649-65) and mental diseases (Orihuela R, McPherson C A, Harry G J. Microglial M1/M2 polarization and metabolic states. Br J Pharmacol. 2016; 173:649-65) and neurodegenerative diseases, including Alzheimer's disease (Nakagawa Y, Chiba K. Diversity and plasticity of microglial cells in psychiatric and neurological disorders. Pharmacol Ther. 2015; 154:21-35) Parkinson's disease (Orihuela R, McPherson C A, Harry G J. Microglial M1/M2 polarization and metabolic states. Br J Pharmacol. 2016; 173:649-65) amyotrophic lateral sclerosis (ALS) and multiple sclerosis. Aging goes in parallel with systemic chronic activation of the immune system and polarization towards a low-level inflammatory status (Ransohoff R M. A polarizing question: do M1 and M2 microglia exist? Nat Neurosci. 2016; 19:987-91; Tang Y, Le W. Differential Roles of M1 and M2 Microglia in Neurodegenerative Diseases. Mol Neurobiol. 2016; 53:1181-94).

### Medicinal Applications of Cannabis

Cannabis sativa L. has a tradition of medical use. Medicinal Cannabis has attracted significant interest due to its anti-inflammatory, anti-oxidative and anti-necrotic protective effects, as well as displaying a favourable safety and tolerability profile in humans, making it a promising candidate in many therapeutic avenues. However, clinical use has been restricted because of untoward effects on the central nervous system and the possibility of abuse and addiction. The plant exudes a resin containing a mix of cannabinoids with two principal components, Δ9-tetrahydrocannabinol (THC) and cannabidiol (CBD). The structure of CBD was described in the 1960s and has garnered attention due to the lack of psychotropic activity. Because of its excellent tolerability in humans, the lack of psychoactive action, and low abuse potential, it seems ideal for clinical trial.

### C. Cannabinoids

In addition to its good safety profile and the lack of psychoactive effects, CBD also presents a wide range of therapeutic effects. Several experimental in vitro and in vivo studies demonstrate anti-inflammation and immune modifying, anti-psychotic, analgesic and anti-epileptic actions. For these reasons, CBD is currently one of the most studied cannabinoids. Compared to Δ9-THC, CBD shows a low affinity for cannabinoid receptor type 1 (CB₁) and type 2 (CB₂). CB₁ receptors are mainly found in the terminals of central and peripheral neurons, and CB₂ receptors primarily in immune cells. Several in vitro studies have shown that CBD, at low concentrations, has weak CB₁ and CB₂ antagonistic effect.

Studies suggest that CBD behaves as a negative allosteric modulator of CB₁, meaning that CBD does not activate the receptor directly but alters the potency and efficacy of CBD1's orthosteric ligands: Δ9-THC and 2-arachidonoylglycerol (2-AG). These preliminary results need further validation but may explain the ability of CBD to antagonise some of the effects of Δ9-THC reported in vitro, in vivo and human clinical studies. It has also been suggested that the role of CBD as an allosteric modulator of CB₁ can explain its therapeutic role in the treatment of central and peripheral nervous system disorders. CBD has also shown to inhibit neutrophil chemotaxis and proliferation. It may also induce arachidonic acid release and reduce prostaglandin E2 (PGE2) and nitric oxide (NO) production.

However, not all physiological effects of CBD are mediated by cannabinoid receptors. CBD has numerous targets outside the endocannabinoid system, and its action independent of the cannabinoid receptor is the subject of recent pharmacological studies. Some effects, such as anti-inflammatory and immunosuppressive action, are mediated by more than one target. The anti-inflammatory, immunosuppressive effects are possibly mediated by activation of adenosine receptors, A_{1A} and A_{2A} and strychnine-sensitive α1 and α1ß glycine receptors and the inhibition of the equilibrative nucleoside transporter. Furthermore, the activity of CBD may elicit different physiological effects from the same target. For example, the same glycine receptor is implicated in both anti-inflammation and suppression of neuropathic pain. While effects on serotonin 5HT1A receptors may generate anxiolytic, panicolytic and antidepressant effects, research has showed an in-depth review of the molecular pharmacology of CBD. Despite advances in the molecular pharmacology of CBD, the many pharmacological mechanisms of CBD remain uncharacterized.

Published studies in animals demonstrated that the oral bioavailability of cannabidiol has been shown to be approximately between 13-19%. Plasma and brain concentrations are dose-dependent in animals, and bioavailability is increased with various oil formulations. Cannabinoids undergo extensive first pass metabolism and its metabolites are mostly excreted via the kidneys.

Cannabinoids are metabolized extensively by the liver, where it is hydroxylated to 7—OH-CBD by P450 enzymes, predominantly by the CYP3A (2/4) and CYP2C (8/9/19) families of isozymes. This metabolite then undergoes significant further metabolism in the liver, and the resulting metabolites are excreted in the faeces and, to a much lesser extent, in the urine.

It is known that cannabidiol acts on cannabinoid (CB) receptors (CB₁ and CB₂) of the endocannabinoid system, which are found in numerous areas of the body, including the peripheral and central nervous systems, including the brain. The endocannabinoid system regulates many physiological responses of the body including pain, memory, appetite, and mood. More specifically, CB₁ receptors can be found within the pain pathways of the brain and spinal cord where they may affect cannabidiol-induced analgesia and anxiolysis, and CB₂ receptors have an effect on immune cells, where they may affect cannabidiol-induced anti-inflammatory processes.

Cannabidiol has been shown to act as a negative allosteric modulator of the cannabinoid CB₁ receptor, the most abundant G-Protein Coupled Receptor (GPCR) in the body. Allosteric regulation of a receptor is achieved through the modulation of the activity of a receptor on a functionally distinct site from the agonist or antagonist binding site. The negative allosteric modulatory effects of cannabidiol are therapeutically important as direct agonists are limited by their psychomimetic effects while direct antagonists are limited by their depressant effects.

There has been some developments in the regulatory approval of CBD. Epidiolex® is a plant-derived, pharmaceutical grade cannabidiol (CBD) medication which attained FDA approval for use in the United States in 2018. Epidiolex® contains 100 mg of cannabidiol per milliliter (mL) of solutions and is taken orally twice daily. The Australian Therapeutic Goods Administration (TGA) approved Epidiolex in September 2020 for the treatment of seizures associated with Lennox-Gastaut syndrome (LGS) or Dravet syndrome in patients two years of age or older

There is a need in the art for improved cannabinoid compositions and effective treatments of paediatric neurological disorders. It is an objective of the invention to overcome one or more problems foreshadowed by the prior art.

### SUMMARY OF THE INVENTION

In a first aspect, the invention broadly resides in a method of treating a paediatric neurological disorder, said method comprising administering to a patient in need thereof a therapeutically effective amount of a composition including about 50 w/w % of CBDA; and wherein all other cannabinoids come to about 15 w/w %.

In a preferred embodiment, the invention comprises a method of treating a paediatric neurological disorder, said method comprising administering to a patient in need thereof a therapeutically effective amount of a composition comprising the following cannabinoids:
w/w %
CBDA 40-60%;
CBD 1-5%;
CBG 1-10%;
CBDP 1-5%;
CBDB 1-5%;
CBGA 1-10%;
CBN 1-3%; and
THC<1%.

In another preferred embodiment, the composition comprises the following cannabinoids:
w/w %
CBDA 50%;
CBD 2%;
CBG 5%;
CBDP 2%;
CBDB 2%;
CBGA 5%;
CBN 1-3%; and
THC<0.3%.

In another preferred embodiment, the composition comprises the following cannabinoids:
w/w %
CBDA 49%;
CBD 2%;
CBG 5%;
CBDP 2%;
CBDB 2%;
CBGA 5%;
CBN 3%; and
THC<0.3%.

In another preferred embodiment, the composition comprises the following cannabinoids:
w/w %
CBDA 45%;
CBD 1%;
CBG 4%;
CBDP 1%;
CBDB 2%;
CBGA 4%;
CBN 2%; and
THC<0.2%.

In another preferred embodiment, the composition comprises the following cannabinoids:
w/w %
CBDA 45%;
CBD 1%;
CBG 4%;
CBDP 1%;
CBDB 2%;
CBGA 4%;
CBN 1%; and
THC<0.2%.

In another preferred embodiment, the composition comprises the following cannabinoid (w/w %): THC (alpha and delta 8) selected from the group consisting of: THC<1%; THC<0.3%; THC<0.2%. THC>1%; THC>1% up to 10%; THC>1% up to 15%; THC>1% up to 30%.

In another preferred embodiment, the composition further comprises cannabinoids selected from the group consisting of: CBC; CBN; CBDV; Plant terpenes; and THC (alpha and delta 8).

In another preferred embodiment, the composition further comprises the following cannabinoids:
CBC;
CBN;
CBDV;
Plant terpenes; and
THC (alpha and delta 8).

In a preferred embodiment, the composition further comprises the following cannabinoids:
w/w %
CBC (1-3%)
CBN (1-3%)
CBDV (1-3%)
Plant terpenes (2-5%)
THC (alpha and delta 8)>1% up to 30%.

In a preferred embodiment, the composition comprises the following cannabinoids:
w/w %
CBDA 40-60%;
CBD 1-5%;
CBG 1-10%;
CBDP 1-5%;
CBDB 1-5%;
CBGA 1-10%;
CBN 1-3%;
CBC 1-3%;
CBN 1-3%;
CBDV 1-3%;
Plant terpenes (2-5%); and
THC<1%.

In a preferred embodiment, the composition comprises the following cannabinoids:
w/w %
CBDA 40-60%;
CBD 1-5%;
CBG 1-10%;
CBDP 1-5%;
CBDB 1-5%;
CBGA 1-10%;
CBN 1-3%;
CBC 1-3%;
CBN 1-3%;
CBDV 1-3%;
Plant terpenes (2-5%); and
THC (alpha and delta 8)>1% up to 30%.

In a preferred embodiment, the composition comprises one or more cannabinoids selected from the group consisting of:
w/w %
CBDA 40-60%;
CBD 1-5%;
CBG 1-10%;
CBDP 1-5%;
CBDB 1-5%;
CBGA 1-10%;
CBN 1-3%;
CBC 1-3%;
CBN 1-3%;
CBDV 1-3%;
Plant terpenes (2-5%); and
THC<1%.

In a preferred embodiment, the composition comprises one or more cannabinoids selected from the group consisting of:
w/w %
CBDA 40-60%;
CBD 1-5%;
CBG 1-10%;
CBDP 1-5%;
CBDB 1-5%;
CBGA 1-10%;
CBN 1-3%;
CBC 1-3%;
CBN 1-3%;
CBDV 1-3%;
Plant terpenes (2-5%); and
THC (alpha and delta 8)>1% up to 30%.

In a preferred embodiment, the composition comprises cannabinoids in amounts selected from the group consisting of any one of the above-mentioned embodiments.

In a preferred embodiment, the paediatric neurological disorder is selected from the group consisting of: PANS/PANDAS, Tourette syndrome, cerebral palsy, ataxia, attention deficit hyperactivity disorder (ADHD), Lennox Gastaut, Dravet syndrome, leukodystrophy and subtypes, Reye syndrome, Rett syndrome, Fragile-X syndrome, Phelan-McDermid syndrome, Angelman syndrome, Pitt-Hopkins syndrome, Prader-Willi syndrome, a metabolic disorder and a glycosylation disorder.

In a preferred embodiment, the PANS is selected from the group consisting of: PANDAS and PITANDS.

In a preferred embodiment, the metabolic disorder is selected from the group consisting of: Hurler syndrome, Niemann-Pick disease, Tay-Sachs disease, Gaucher disease, Fabry disease and Krabbe disease, Galactosemia, Maple syrup urine disease, Phenylketonuria, glycogen storage disease, mitochondrial disorders, Friedreich ataxia, Peroxisomal disorder, metal metabolism disorder, organic acidemias and urea cycle disorders.

In a preferred embodiment, the glycosylation disorder is selected from the group consisting of: disorders of N-glycosylation, disorders of O-glycosylation, disorders of glycosylphosphatidylinositol (GPI) anchor and glycolipid anchor, and disorders of glycosylation pathways.

In a preferred embodiment, the paediatric neurological disorder is associated with neuroinflammation.

In a preferred embodiment, the composition further comprises an oil selected from the group consisting of: a synthetic oil; plant based oil; mineral oil; canola oil; and olive oil.

In a preferred embodiment, the composition comprises less than 5% w/w terpenes.

In a preferred embodiment, the composition comprises less than 2% w/w organic plant material.

In a preferred embodiment, the composition comprises less than 2% w/w of plant phenols.

In a preferred embodiment, the cannabinoid component of the composition is selected from the group consisting of: between 1 and 500 mg/ml; between 10 and 100 mg/ml; be at a concentration of 50 mg/ml.

In a preferred embodiment, the CBDA component of the composition is selected from the group consisting of: between 1 and 500 mg/ml; between 10 and 100 mg/ml; be at a concentration of 50 mg/ml.

In a preferred embodiment, the composition has a UPLC mass chromatogram corresponding to FIG. 3 utilising the conditions described in Example 1.

In a preferred embodiment, the composition further comprises an additional active ingredient.

In a preferred embodiment, the additional active ingredient is selected from the group consisting of: diclofenac, prednisone, celecoxib and psylocibin.

In a second aspect, the invention is a pharmaceutical composition adapted for treating a paediatric neurological disorder comprising the composition discussed under the first aspect of the invention together with a pharmaceutically acceptable carrier.

In a third aspect, the invention is a dosage form adapted for treating a paediatric neurological disorder comprising the composition discussed under the first aspect of the invention.

In a preferred embodiment, the dosage form adapted for treating a paediatric neurological disorder comprises the following composition:
w/w %
CBDA 40-60%;
CBD 1-5%;
CBG 1-10%;
CBDP 1-5%;
CBDB 1-5%;
CBGA 1-10%;
CBN 1-3%
THC<1%; and
wherein the CBDA component of the composition is selected from the group consisting of: between 1 mg and 1000 mg; between 1 mg and 500 mg; between 1 and 100 mg; less than 400 mg; less than 300 mg; less than 200 mg and less than 100 mg.

In a preferred embodiment, the dosage form adapted for treating a paediatric neurological disorder comprises the following composition:
w/w %
CBDA 40-60%;
CBD 1-5%;
CBG 1-10%;
CBDP 1-5%;
CBDB 1-5%;
CBGA 1-10%;
CBN 1-3%
THC<1%; and
wherein the CBDA component of the composition is selected from the group consisting of: 600 mg; 400 mg; 300 mg; 200 mg; 100 mg; 50 mg; 10 mg; 5 mg; 2 mg; 1 mg.

In a fourth aspect, the invention is a kit adapted for treating a paediatric neurological disorder, said kit comprising the following composition:
w/w %
CBDA 40-60%;
CBD 1-5%;
CBG 1-10%;
CBDP 1-5%;
CBDB 1-5%;
CBGA 1-10%;
CBN 1-3%
THC<1%; and
together with instructions for use.

In a fifth aspect, the invention is the use of a composition in the manufacture of a medicament for the treatment of a paediatric neurological disorder, wherein the composition comprises the following cannabinoids:
w/w %
CBDA 40-60%;
CBD 1-5%;
CBG 1-10%;
CBDP 1-5%;
CBDB 1-5%;
CBGA 1-10%;
CBN 1-3%; and
THC<1%.

In a preferred embodiment, the cannabinoids are present in amounts selected from the group consisting of:
Composition 1 comprising
w/w %
CBDA 50%;
CBD 2%;
CBG 5%;
CBDP 2%;
CBDB 2%;
CBGA 5%;
CBN 3% and
THC<0.3%;
and
Composition 2 comprising
w/w %
CBDA 45%;
CBD 1%;
CBG 4%;
CBDP 1%;
CBDB 2%;
CBGA 4%;
CBN 2% and
THC<0.2%.

In a preferred embodiment, the paediatric neurological disorder is selected from the group consisting of: PANS/PANDAS, Tourette syndrome, cerebral palsy, ataxia, attention deficit hyperactivity disorder (ADHD), Lennox Gastaut, Dravet syndrome, leukodystrophy and subtypes, Reye's syndrome, Rett syndrome, Fragile-X syndrome, Phelan-McDermid syndrome, Angelman syndrome, Pitt-Hopkins syndrome, Prader-Willi syndrome, metabolomic disorders and glycosylation disorders.

Preferably, the PANS is selected from the group consisting of: PANDAS and PITAND.

In a preferred embodiment, the metabolic disorder is selected from the group consisting of: Hurler syndrome, Niemann-Pick disease, Tay-Sachs disease, Gaucher disease, Fabry disease and Krabbe disease, Galactosemia, Maple syrup urine disease, Phenylketonuria, glycogen storage disease, mitochondrial disorders, Friedreich ataxia, Peroxisomal disorder, metal metabolism disorder, organic acidemias and urea cycle disorders.

In a preferred embodiment, the glycosylation disorder is selected from the group consisting of: disorders of N-glycosylation, disorders of O-glycosylation, disorders of glycosylphosphatidylinositol (GPI) anchor and glycolipid anchor and disorders of glycosylation pathways.

In a preferred embodiment, the paediatric neurological disorder is associated with neuroinflammation.

In a preferred embodiment, the composition further comprises an oil selected from the group consisting of: a synthetic oil; plant based oil; mineral oil; canola oil; and olive oil.

In a preferred embodiment, the composition comprises less than 5% w/w terpenes.

In a preferred embodiment, the composition comprises less than 2% w/w organic plant material.

In a preferred embodiment, the composition comprises less than 2% w/w of plant phenols.

In a preferred embodiment, the cannabinoid component of the composition is selected from the group consisting of: between 1 and 500 mg/ml; between 10 and 100 mg/ml; be at a concentration of 50 mg/ml.

In a preferred embodiment, the CBDA component of the composition is selected from the group consisting of: between 1 and 500 mg/ml; between 10 and 100 mg/ml; be at a concentration of 50 mg/ml.

In a preferred embodiment, the composition has a UPLC mass chromatogram corresponding to FIG. 3 utilising the conditions described in Example 1.

In a preferred embodiment, the composition further comprises an additional active ingredient.

In a preferred embodiment, the additional active ingredient is selected from the group consisting of: diclofenac, prednisone, celecoxib and psylocibin.

In a sixth aspect, the invention is a process of extracting the composition of the invention from Cannabis plant material, said process comprising the steps of:
1) Grinding the Cannabis plant material to a sufficient grind size;
2) Contacting the grind produced by step a) with oil;
3) Mixing the grind and oil for a sufficient time period to form a mixture;
4) Pressing the mixture to reclaim the oil;
5) Centrifuging the oil to further refine the oil; and
6) Collecting the oil extract in a suitable container/steel vessel.

In a seventh aspect, the invention is a process of extracting the composition of the invention from Cannabis plant material, said process comprising the steps of:
1) Grinding the Cannabis plant material to a sufficient grind size;
2) Contacting the grind produced by step a) with an alcohol;
3) Mixing the grind and alcohol for a sufficient time period to form a mixture;
4) Sonicating the mixture;
5) Centrifuging the mixture; and
6) Collecting the alcohol extract in a suitable container.

In an eighth aspect, the invention includes a composition, method or process as described by the examples following.

Further features of the present invention are more fully described in the following description of several non-limiting embodiments thereof. This description is included solely for the purposes of exemplifying the present invention. It should not be understood as a restriction on the broad summary, disclosure or description of the invention as set out above.

### BRIEF DESCRIPTION OF THE DRAWINGS

Below is a brief description of each of the figures and drawings.

FIG. 1 is a UPLC mass chromatogram of the cannabinoid standard mixture (10 ppm each) in; a) positive; and b) negative ionization mode. It is a LC-MS chromatogram cannabinoid standard mixture (10 ppm each) in a) positive and b) negative ionization mode

FIG. 2 is an in-source fragmentation of CBD and CBG from the reference solution.

FIG. 3 presents a mass spectrometry chromatograms of NTI164.

FIG. 4 presents a mass spectrometry chromatograms of CBD Variants.

FIG. 5 presents the expression of inflammation, including the induction of iNOS in neuroronal cells.

FIG. 6 presents the neuronal viability quantified using MTT [3-(4,5-dimethylthiazol-2-yl-)-2,5-diphenyl-2H-tetrazolium bromide].

FIG. 7 demonstrates that NTI164 stimulates the maturation of immature neurons into healthy cells even without the presence of any glutamate induced insult.

FIG. 8 demonstrates that CBD is toxic in this paradigm while NTI164 is non-toxic and has positive effects on cell number and cell viability.

FIG. 9 shows the microglial responses under inflammatory conditions assessing arginase 1 expressions.

FIG. 10 is a diagram which outlines the arginine metabolism and the effects it has on the overall balance of anti-inflammatory and pro-inflammatory signals. (Reference: Review. Gonçalo S. Clemente, Aren van Waarde, Ines F. Antunes, Alexander Domling and Philip H. Elsinga. Arginase as a Potential Biomarker of Disease Progression: A Molecular Imaging Perspective. (2020)).

FIG. 11 shows the distribution of patients actively using NTI164 for Example 10.

FIG. 12 show the distribution of the severity of illness of active patients at baseline as per CGI-S severity of illness for Example 10.

FIG. 13 shows the maximum tolerated dose for active patients for Example 10.

FIG. 14 shows the CGI-S global improvement at 28 days of NTI164 treatment.

FIG. 15 shows the CGI-S severity of illness after 28 days of treatment.

FIG. 16 shows the CGI-S severity of illness after 28 days of treatment.

FIG. 17 shows the CGI-S therapeutic effect after 28 days of treatment.

FIG. 18 shows heat maps depicting the top 1000 differentially expressed genes in the whole blood transcriptomes of children with atypical OCD compared to controls. There is a baseline difference in gene expression profile between controls (pink bar) and atypical OCD pre-IVIG (purple bar). Comparing pre-IVIG (purple bar) with post-IVIG (blue, green bars) shows that IVIG can modify the gene expression profile but by 28 days this effect wanes. (Reference: Antistreptolysin-O titers: implications for adult PANDAS. Pediatric autoimmune neuropsychiatric disorders associated with streptococcal infections. Church A J, Dale R C. Am J Psychiatry. 2002 February; 159 (2): 320).

FIG. 19 shows gene ontology pathway analysis showing the top 10 enriched pathways in the whole blood transcriptomes of children with pediatric acute-onset neuropsychiatric syndrome (PANS) compared to controls. Immune pathways (e.g., neutrophil degranulation, neutrophil activation) are downregulated (blue) while gene translation pathways (e.g., protein targeting to ER, translation initiation) are upregulated (red). Abbreviations: ER, endoplasmic reticulum (Reference: Shekeeb S Mohammad and Russell C Dale. Principles and approaches to the treatment of immune-mediated movement disorders. Eur J Paediatr Neurol. 2018 March; 22 (2): 292-300).

FIG. 20 shows the age distribution of patients actively using NTI164 for Example 12.

FIG. 21 show the distribution of the severity of illness of active patients at baseline as per CGI-S severity of illness for Example 12. CGI-S refers to the Clinical Global Impression Scale-Severity of Illness.

FIG. 22 shows the CGI-S global improvement at 20 weeks of NTI164 treatment.

FIG. 23 shows the CGI-S global improvement over time up to and including 20 weeks of NTI164 treatment.

FIG. 24 shows the CGI-S severity of illness at 20 weeks of treatment.

FIG. 25 shows the CGI-S severity of illness over time up to and including 20 weeks of treatment.

FIG. 26 shows the CGI-S severity of illness at 20 weeks of treatment.

FIG. 27 shows the CGI-S therapeutic effect over time up to and including 20 weeks of treatment.

FIG. 28 shows the CGI-S therapeutic effect at 20 weeks of treatment.

FIG. 29 shows the CGI-S severity of illness over time up to and including 52 weeks of treatment. P=0.03.

FIG. 30 shows the mean severity of illness over time up to and including 52 weeks of treatment. NTI164 treatment is associated with a significant reduction in disease severity (1.3 scale change, 30% improvement). Approximately 40% of subjects were markedly or severely ill at baseline-0% from week 4 onwards.

FIG. 31 shows CGI-S global improvement over time up to and including 52 weeks of NTI164 treatment. 100% of active patients showed improvement after 20 weeks of daily treatment with NTI164. After 52 weeks of daily treatment with NTI164, 90% of active patients (n=10) had a global improvement of Much Improved and 10% (n=1) had a score of Very Much Improved.

FIG. 32 shows the CGI-S therapeutic effect over time up to and including 52 weeks of treatment. After 52 weeks of daily NTI164 treatment, 10% of active patients demonstrated the highest possible efficacy index of: 1 Markeed therapeutic effect-Vast Improvement. Complete or nearly complete remission of all symptoms. 90% of patients had an efficacy indes of either 5 or 6: Moderate therapeutic effect-Decided improvement. Partial remission of symptoms.

FIG. 33 shows the Revised Children's Anxiety and Depression Scale, Parent-rated (RCADS-P) scale, Total Score mean change over 12 weeks.

FIG. 34 shows the Clinical Global Impression-Severity (CGI-S) scale, Severity of Illness mean changes over 12 weeks.

FIG. 35 shows the Clinical Global Impression-Severity (CGI-S) scale, Severity of Illness rating over 12 weeks.

FIG. 36 shows the Clinical Global Impression-Severity (CGI-S) scale, Therapeutic Effect over 12 weeks.

FIG. 37 shows the Clinical Global Impression-Improvement (CGI-I) scale, Impression of Improvement over time.

FIG. 38 shows the Yale Global Tic Severity Scale (YGTSS) scale, Total Score mean change over time.

FIG. 39 shows the Children's Yale-Brown Obsessive-Compulsive Scale (CY-BOCS) scale, Total Score mean changes over time.

FIG. 40 shows the Conner's Scale (Assessment of Attention Deficit Hyperactivity Disorder (ADHD) Behaviours) scale, Total Score mean changes over time.

FIG. 41 shows the EQ-5D-Y (Standardised measure of health-related quality of life domains) scale, Total Score mean changes over time.

### DETAILED DESCRIPTION OF THE INVENTION

For convenience, the following sections generally outline the various meanings of the terms used herein. Following this discussion, general aspects regarding compositions, use of medicaments and methods of the invention are discussed, followed by specific examples demonstrating the properties of various embodiments of the invention and how they can be employed.

Those skilled in the art will appreciate that the invention described herein is susceptible to variations and modifications other than those specifically described. The invention includes all such variations and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness. None of the cited material or the information contained in that material should, however be understood to be common general knowledge.

Manufacturer's instructions, descriptions, product specifications, and product sheets for any products mentioned herein or in any document incorporated by reference herein, are hereby incorporated herein by reference, and can be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

### 1. DEFINITIONS

The meaning of certain terms and phrases used in the specification, examples, and appended claims, are provided below. If there is an apparent discrepancy between the usage of a term in the art and its definition provided herein, the definition provided within the specification shall prevail.

Other than in the operating examples, or where otherwise indicated, all numbers expressing quantities of ingredients or reaction conditions used herein should be understood as modified in all instances by the term “about.” The term “about” when used in connection with percentages can mean±1%.

The invention described herein may include one or more range of values (e.g., size, concentration etc.). A range of values will be understood to include all values within the range, including the values defining the range, and values adjacent to the range which lead to the same or substantially the same outcome as the values immediately adjacent to that value which defines the boundary to the range. For example, a person skilled in the field will understand that a 10% variation in upper or lower limits of a range can be totally appropriate and is encompassed by the invention. More particularly, the variation in upper or lower limits of a range will be 5% or as is commonly recognised in the art, whichever is greater.

In this application, the use of the singular also includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

Throughout this specification, unless the context requires otherwise, the word “comprise” or variations such as “comprises” or “comprising”, will be understood to imply the inclusion of a stated integer or group of integers but not the exclusion of any other integer or group of integers.

“Pediatric age” as used herein is consistent with its use by The Federal Food, Drug, and Cosmetic Act (FD&C Act). The Federal Food, Drug, and Cosmetic Act (FD&C Act) defines pediatric patients as persons aged 21 or younger at the time of their diagnosis or treatment. Pediatric subpopulations are further categorized as follows: Neonates—from birth through the first 28 days of life. Infants-29 days to less than 2 years. (https://www.fda.gov/medical-devices/products-and-medical-procedures/pediatric-medical-devices).

“Therapeutically effective amount” as used herein with respect to methods of treatment and in particular drug dosage, shall mean that dosage that provides the specific pharmacological response for which the drug is administered in a significant number of subjects in need of such treatment. It is emphasized that “therapeutically effective amount,” administered to a particular subject in a particular instance will not always be effective in treating the diseases described herein, even though such dosage is deemed a “therapeutically effective amount” by those skilled in the art. It is to be further understood that drug dosages are, in particular instances, measured as oral dosages, or with reference to drug levels as measured in blood. Amounts effective for such a use will depend on: the desired therapeutic effect; the potency of the biologically active material; the desired duration of treatment; the stage and severity of the disease being treated; the weight and general state of health of the patient; and the judgment of the prescribing physician. Treatment dosages need to be titrated to optimize safety and efficacy. One skilled in the art will appreciate that the appropriate dosage levels for treatment will thus vary depending, in part, upon the indication for which the active agent is being used, the route of administration, and the size (body weight, body surface or organ size) and condition (the age and general health) of the patient. Accordingly, the clinician may titre the dosage and modify the route of administration to obtain the optimal therapeutic effect. A typical dosage may range from about 0.1 μg/kg to up to about 100 mg/kg or more, depending on the factors mentioned above. In other embodiments, the dosage may range from 0.1 μg/kg up to about 100 mg/kg; or 1 μg/kg up to about 100 mg/kg; or 5 μg/kg up to about 100 mg/kg.

The frequency of dosing will depend upon the pharmacokinetic parameters of the active agent and the formulation used. Typically, a clinician will administer the composition until a dosage is reached that achieves the desired effect. The composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data.

As used herein “pharmaceutically acceptable carrier” includes any and all solvents, dispersion media, coatings, antibacterial and antifungal agents, isotonic and absorption delaying agents, and the like that are physiologically compatible.

As used herein the term “subject” generally includes mammals such as: humans; farm animals such as sheep, goats, pigs, cows, horses, llamas; companion animals such as dogs and cats; primates; birds, such as chickens, geese and ducks; fish; and reptiles. The subject is preferably human. In one embodiment, the human subject is an infant, child, or adolescent. In one embodiment, the human subject is a paediatric patient and aged 21 or younger at the time of their diagnosis or treatment.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Features of the invention will now be discussed with reference to the following non-limiting description and examples.

### 2. EMBODIMENTS

### Composition

The present invention provides a composition comprising the following cannabinoids:
about 50 w/w % of CBDA; and
wherein all other cannabinoids come to about 15 w/w %.

In a preferred embodiment, the invention provides a composition comprising the following cannabinoids:
w/w % about 50% of CBDA; and
about 2% CBD.

In a further preferred embodiment, the invention provides a composition comprising the following cannabinoids:
w/w % about 50% of CBDA; and
about 5% CBG.

In a further preferred embodiment, the invention provides a composition comprising the following cannabinoids:
w/w %
about 50% of CBDA; and
about 2% CBDP.

In a further preferred embodiment, the invention provides a composition comprising the following cannabinoids:
w/w %
about 50% of CBDA; and
about 2% CBDB.

In a further preferred embodiment, the invention provides a composition comprising the following cannabinoids:
w/w %
about 50% of CBDA; and
about 5% CBGA.

In a further preferred embodiment, the invention provides a composition comprising cannabinoids, wherein the ratio of CBDA to all other cannabinoids is between 4:1 and 2:1.

In a further preferred embodiment, the invention provides a composition comprising cannabinoids, wherein the ratio of CBDA to all other cannabinoids is about 3:1.

In a further preferred embodiment, the invention provides a composition comprising cannabinoids, wherein the ratio of CBDA to all other cannabinoids is about 3.21:1.

In a further preferred embodiment, the invention provides a composition comprising the following cannabinoids:
w/w %
CBDA 40-60%;
CBD 1-5%;
CBG 1-10%;
CBDP 1-5%;
CBDB 1-5%;
CBGA 1-10%;
CBN 1-3%; and
THC<1%.

In a further preferred embodiment, the invention provides a composition comprising the following cannabinoids:
w/w %
CBDA 45-55%;
CBD 1-3%;
CBG 3-7%;
CBDP 1-3%;
CBDB 1-3%;
CBGA 3-7%;
CBN 1-3%; and
THC<0.5%.

In a further preferred embodiment, the invention provides a composition comprising the following cannabinoids:
w/w %
CBDA 50%;
CBD 2%;
CBG 5%;
CBDP 2%;
CBDB 2%;
CBGA 5%;
CBN 3%; and
THC<0.3%.

In a further preferred embodiment, the invention provides a composition comprising the following cannabinoids:
w/w %
CBDA 49%;
CBD 2%;
CBG 5%;
CBDP 2%;
CBDB 2%;
CBGA 5%;
CBN 2%; and
THC<0.3%.

In a further preferred embodiment, the invention provides a composition comprising the following cannabinoids:
w/w %
CBDA 48.78%;
CBD 1.89%;
CBG 4.88%;
CBDP 1.68%;
CBDB 1.76%;
CBGA 4.76%;
CBN 1%; and
THC<0.18%.

In a further preferred embodiment, the invention provides a composition comprising the following cannabinoids:
w/w %
CBDA 45%;
CBD 1%;
CBG 4%;
CBDP 1%;
CBDB 2%;
CBGA 4%;
CBN 2%; and
THC<0.2%.

In a further preferred embodiment, the invention provides a composition comprising the following cannabinoids:
w/w %
CBDA 45.28%;
CBD 1.39%;
CBG 3.88%;
CBDP 1.18%;
CBDB 1.56%;
CBGA 3.76%;
CBN 1%; and
THC<0.18%.

In a further preferred embodiment, the invention provides a composition comprising the following cannabinoids:
w/w %
CBDA 62.78%;
CBD 5.80%;
CBG 0.44%;
CBGA 1.26%;
CBN 1.98%; and
THC<0.70%.

In a further preferred embodiment, the invention provides a composition comprising the following cannabinoids:
w/w %
CBDA 60.29%;
CBD 5.34%;
CBG 0.39%;
CBGA 1.14%;
CBN 0.85%; and
THC<0.65%.

In a further preferred embodiment, the invention provides a composition wherein the cannabinoids are present in amounts selected from the group consisting of:
Composition 1 comprising
w/w %
CBDA 50%;
CBD 2%;
CBG 5%;
CBDP 2%;
CBDB 2%;
CBGA 5%;
CBN 3%; and
THC<0.3%;
and
Composition 2 comprising
w/w %
CBDA 45%;
CBD 1%;
CBG 4%;
CBDP 1%;
CBDB 2%;
CBGA 4%;
CBN 2%; and
THC<0.2%.

In a further preferred embodiment, the invention provides a composition, wherein the quantity of the cannabinoids is determined by a method selected from the group consisting of: high performance chromatography (HPLC), proton nuclear magnetic resonance spectroscopy (H1 NMR); and mass spectrometry.

In a further preferred embodiment, the invention provides a composition derived from Cannabis plant material.

In a further preferred embodiment, the invention provides a composition wherein the said listed cannabinoids are synthetic.

In a further preferred embodiment, the invention provides a composition wherein the said listed cannabinoids are a mixture of plant derived and synthetic cannabinoids.

In a further preferred embodiment, the invention provides a composition further comprising an oil selected from the group consisting of: a synthetic oil; plant based oil; mineral oil; canola oil; and olive oil.

In a further preferred embodiment, the composition comprises less than 5% w/w terpenes.

In a further preferred embodiment, the composition comprises less than 2% w/w organic plant material.

In a further preferred embodiment, the composition comprises less than 2% w/w of plant phenols.

In a further preferred embodiment, the composition comprises components selected from the group consisting of: flavonoids, proteins, sterols and esters.

In a further preferred embodiment, the composition is substantially pure. Preferably, the purity is determined by a method selected from the group consisting of: high performance chromatography (HPLC), proton nuclear magnetic resonance spectroscopy (H1 NMR); and mass spectrometry. Preferably, the purity is selected from the group consisting of: greater than 75% purity; greater than 80% purity; greater than 85% purity; greater than 90% purity; greater than 95% purity; greater than 96% purity; greater than 97% purity; greater than 98% purity; greater than 99% purity; greater than 99.5% purity; greater than 99.6% purity; greater than 99.7% purity; greater than 99.8% purity; greater than 99.9% purity; greater than 99.95% purity; greater than 99.96% purity; greater than 99.97% purity; greater than 99.98% purity and greater than 99.99% purity.

In a further preferred embodiment, the composition comprises less than 0.1 wt % organic impurities as measured a method selected from the group consisting of: high performance chromatography (HPLC), proton nuclear magnetic resonance spectroscopy (H1 NMR); and mass spectrometry.

In a further preferred embodiment, the composition is substantially free of atmospheric oxygen.

In a further preferred embodiment, the composition is sterile. In an alternative preferred embodiment, the composition is not sterile.

In a further preferred embodiment, the invention provides a composition wherein the cannabinoid component of the composition is selected from the group consisting of: between 1 and 500 mg/ml; between 10 and 100 mg/ml; be at a concentration of 50 mg/ml.

In a further preferred embodiment, the invention provides a composition wherein the CBDA component of the composition is selected from the group consisting of: between 1 and 500 mg/ml; between 10 and 100 mg/ml; be at a concentration of 50 mg/ml.

In a further preferred embodiment, the composition is a liquid.

In a further preferred embodiment, the composition is an oil.

In a further preferred embodiment, the composition demonstrates no cannabinoid degradation or decarboxylation when measured at a time point selected from the group consisting of: at 1 day; at 2 days; at 7 days; at 14 days; at 28 days; at 5 weeks; at 6 weeks; and 32 weeks.

In a further preferred embodiment, the composition demonstrates cannabinoid stability when measured at a time point selected from the group consisting of: at 1 day; at 2 days; at 7 days; at 14 days; at 28 days; at 5 weeks; at 6 weeks; and at 32 weeks.

In a further preferred embodiment, the composition demonstrates no mutagenicity, carcinogenicity or genotoxicity when delivered at a concentration that delivers 120 mg/ml of CBDA.

In a further preferred embodiment, the composition is adapted to suppress the activity of any one of the following biomarkers: COX-2; iNOS; TNF-alpha; IL-2; IL-12 and GS-MCF.

Preferably, the composition is adapted to suppress neuroinflammation. More preferably, the composition is adapted for the treatment of a neurological disorder. More preferably, the composition is adapted for the treatment of a paediatric neurological disorder.

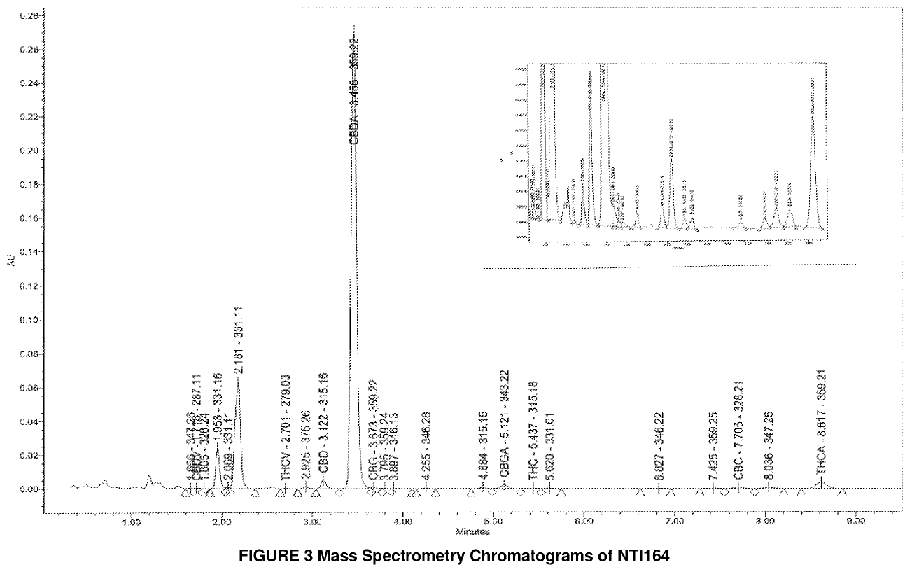

In a further preferred embodiment, the invention provides a composition having a UPLC mass chromatogram corresponding to FIG. 3 utilising the conditions described in Example 1.

In a further preferred embodiment, the composition comprises an additional active ingredient.

Preferably, the additional active ingredient is selected from the group consisting of: a polypeptide; an antibody; a NSAID; a neuro-regulator; and a neurotransmitter, steroids
all relevant classes (corticosteroids), analgesics, anti-psychotics, anti-depressants, immuno-therapy.

In one preferred embodiment, the NSAID is selected from the group consisting of: aspirin, ibuprofen, naproxen, diclofenac, celecoxib, ketorolac, meloxicam, esomeprazole, naproxen, diclofenac, misoprostol, nabumetone, indomethacin, mefenamic acid, etodolac, piroxicam, ketoprofen, diflunisal, oxaprozin, flurbiprofen, sulindac, tolmetin, prednisolone and fenoprofen.

In one embodiment, the additional active ingredient is selected from the group consisting of: diclofenac, prednisone, celecoxib and psylocibin.

In a further preferred embodiment, the ratio of cannabinoid component and the additional active ingredient is selected from the group consisting of: 1 unit w/w of cannabinoid: 1 unit w/w/of the additional active ingredient; 2:1; 3:1; 4:1; 5:1; between 10,000:1 and 1:1; between 1,000:1 and 1:1; between 500:1 and 1:1; between 100:1 and 1:1; between 50:1 and 1:1; and between 10:1 and 1:1.

In a further preferred embodiment, the ratio of the additional active ingredient and cannabinoid is selected from the group consisting of: 1 unit w/w of the additional active ingredient and 1 unit w/w/of the cannabinoid; 2:1; 3:1; 4:1; 5:1; between 10,000:1 and 1:1; between 1,000:1 and 1:1; between 500:1 and 1:1; between 100:1 and 1:1; between 50:1 and 1:1; and between 10:1 and 1:1.

In a further preferred embodiment, the ratio of CBDA and the additional active ingredient is selected from the group consisting of: 1 unit w/w of CBDA: 1 unit w/w/of the additional active ingredient; 2:1; 3:1; 4:1; 5:1; between 10,000:1 and 1:1; between 1,000:1 and 1:1; between 500:1 and 1:1; between 100:1 and 1:1; between 50:1 and 1:1; and between 10:1 and 1:1.

In a further preferred embodiment, the ratio of the additional active ingredient and CBDA is selected from the group consisting of: 1 unit w/w of the additional active ingredient and 1 unit w/w/of the CBDA; 2:1; 3:1; 4:1; 5:1; between 10,000:1 and 1:1; between 1,000:1 and 1:1; between 500:1 and 1:1; between 100:1 and 1:1; between 50:1 and 1:1; and between 10:1 and 1:1.

In one preferred embodiment, the neuroregulator is a psychedelic substance.

Preferably, the neuroregulator is selected from the group consisting of: 3,4-methylenedioxymethamphetamine; lysergic acid diethylamide; and psilocybin.

In a further preferred embodiment, the composition demonstrates synergistic biological activity.

In a further preferred embodiment, the composition demonstrates a level of biological activity that is greater than the sum of: (1) the biological activity of the cannabinoid component when delivered in absence of the additional active ingredient; and (2) the biological activity of the additional active ingredient when delivered in absence of the cannabinoid component.

In a further preferred embodiment, the biological activity is selected from the group consisting of: suppressing inflammation; suppressing neuroinflammation; treating a neurological disorder; suppressing the activity of COX-2; suppressing the activity of iNOS; suppressing the activity of TNF-alpha; suppressing the activity of IL-2; suppressing the activity of IL-12 and suppressing the activity of GS-MCF.

In a further preferred embodiment, the composition is selected from the group consisting of: a therapeutic composition; a pharmaceutical composition; a cosmetic composition; and a veterinary composition.

### Pharmaceutical Compositions

The present invention also provides a pharmaceutical composition comprising the composition of the invention together with a pharmaceutically acceptable carrier.

Therapeutic compositions are within the scope of the present invention.

Preferably the compositions are combined with a pharmaceutically acceptable carrier or diluent to produce a pharmaceutical composition (which may be for human or animal use). Suitable carriers and diluents include isotonic saline solutions, for example phosphate-buffered saline. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. See, e.g., Remington's Pharmaceutical Sciences, 2th Ed. (1995, Mack Publishing Co., Easton, Pa.) which is herein incorporated by reference.

The pharmaceutical composition can contain formulation materials for modifying, maintaining or preserving, for example, the pH, osmolarity, viscosity, clarity, colour, isotonicity, odour, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulphite or sodium hydrogen-sulphite, Vitamin E, Vitamin E phosphate-lipid soluble vitamins, nano emulsions); buffers (such as borate, bicarbonate, tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin), fillers; monosaccharides, disaccharides; and other carbohydrates (such as glucose, mannose, or dextrins); proteins (such as serum albumin, gelatin or immunoglobulins); colouring, flavouring (natural and natural derived products) and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols- and artificial sweetners (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapol); stability enhancing agents (sucrose or sorbitol); tonicity enhancing agents (such as alkali metal halides, preferably sodium or potassium chloride), delivery vehicles, diluents, excipients and/or pharmaceutical adjuvants.

The optimal pharmaceutical composition will be determined by one skilled in the art depending upon, for example, the intended route of administration, delivery format, and desired dosage. Such compositions may influence the physical state, stability, rate of in vivo release, and rate of in vivo clearance of the composition of the invention. The preferred form of the pharmaceutical composition depends on the intended mode of administration and therapeutic application.

The primary vehicle or carrier in a pharmaceutical composition is aqueous and non-aqueous in nature. For example, a suitable vehicle or carrier may be water for injection, physiological saline solution, possibly supplemented with other materials. Neutral buffered saline or saline mixed with serum albumin are further exemplary vehicles. Other exemplary pharmaceutical compositions comprise tris buffer of about pH 7.0-8.5, or acetate buffer of about pH 4.0-5.5, which may further include sorbitol or a suitable substitute thereof. In one embodiment of the present invention, pharmaceutical compositions may be prepared for storage by mixing the selected composition having the desired degree of purity with optional formulation agents in the form an aqueous solution and non-aqueous.

The formulation components are present in concentrations that are acceptable to the site of administration. For example, buffers are used to maintain the composition at physiological pH or at a slightly lower pH, typically within a pH range of from about 5 (4.5) to about 8

Additional pharmaceutical compositions will be evident to those skilled in the art, including formulations of the invention in sustained- or controlled-delivery formulations. Techniques for formulating a variety of other sustained- or controlled-delivery means, such as liposome carriers, bio-erodible microparticles or porous beads and depot injections, are also known to those skilled in the art. Additional examples of sustained-sustained-release preparations include semipermeable polymer matrices in the form of shaped articles, for example, films, or microcapsules. Sustained release matrices may include polyesters, hydrogels, polylactides, copolymers of L-glutamic acid and gamma ethyl-L-glutamate, ethylene vinyl acetate or poly-D(−)-3-hydroxybutyric acid. Sustained-release compositions may also include liposomes, which can be prepared by any of several methods known in the art.

The pharmaceutical composition to be used for in vivo administration typically must be sterile. This may be accomplished by filtration through sterile filtration membranes. In addition, the compositions generally are placed into a container having a sterile access port. Once the pharmaceutical composition has been formulated, it may be stored in sterile vials as a solution.

In yet a further preferred embodiment, the composition retains its effective biological activity for a period selected from the group consisting of; greater than 24 hours; greater than 36 hours; and greater than 48 hours. Preferably, the composition is stable for periods selected from the group consisting of: 6 months, 1 year and 2 years. In one example, the composition is stable at temperatures selected from the group consisting of: −4 °C, 4 °C, 18 °C and 25 °C.

### Dosage Form

Dosage forms are within the scope of the invention. In a preferred embodiment, the invention provides a dosage form comprising the composition as described in the first aspect of this invention.

Preferably, the cannabinoid component of the composition of the dosage form is selected from the group consisting of: between 1 mg and 1000 mg; between 1 mg and 500 mg; between 1 and 100 mg; less than 400 mg; less than 300 mg; less than 200 mg and less than 100 mg. More preferably the cannabinoid component of the composition is selected from the group consisting of: 600 mg; 400 mg; 300 mg; 200 mg; 100 mg; 50 mg; 10 mg; 5 mg; 2 mg; 1 mg. Preferably the CBDA component of the composition of the dosage form is selected from the group consisting of: between 1 mg and 1000 mg; between 1 mg and 500 mg; between 1 and 100 mg; less than 400 mg; less than 300 mg; less than 200 mg and less than 100 mg. More preferably, the CBDA component of the composition is selected from the group consisting of: 600 mg; 400 mg; 300 mg; 200 mg; 100 mg; 50 mg; 10 mg; 5 mg; 2 mg; 1 mg.

In a further embodiment, the dosage form is form selected from the group consisting of: a solution, tablet, capsule, wafer, dry power sachet and vial/freeze-dried Preferably, the dosage form is stored in a sealed and sterile container.

### Method for Treating

The invention also provides a method of treating a disorder, said method comprising administering to a patient in need thereof a therapeutically effective amount of the dosage form of the invention.

In a further preferred embodiment, the dosage form is administered at an amount to at least partially treat the disorder.

In a further preferred embodiment, the therapeutically effective amount is an amount of cannabinoid selected from the group consisting of: between 1 to 100 mg/kg/day; between 2 and 50 mg/kg/day; between 5 and 40 mg/kg/day; between 10 and 30 mg/kg/day; between 20 and 25 mg/kg/day; and 20 mg/kg/day. Preferably, the therapeutically effective amount is an amount of cannabinoid vis selected from the group consisting of: 10 mg/day; 15 mg/day; 40 mg/day; 400 mg/day; 600 mg/day; 800 mg/day; 1280 mg/day; 1500 mg/day.

In a further preferred embodiment, therapeutically effective amount is an amount of CBDA selected from the group consisting of: between 1 to 100 mg/kg/day; between 2 and 50 mg/kg/day; between 5 and 40 mg/kg/day; between 10 and 30 mg/kg/day; between 20 and 25 mg/kg/day; and 20 mg/kg/day. Preferably, the therapeutically effective amount is an amount of CBDA vis selected from the group consisting of: 10 mg/day; 15 mg/day; 40 mg/day; 400 mg/day; 600 mg/day; 800 mg/day; 1280 mg/day; 1500 mg/day.

In a further preferred embodiment, Tmax occurs between 1 and 4 hours.

In a further preferred embodiment, T1/2 occurs between 1.1 and 2.4 hours.

In a further preferred embodiment, the therapeutically effective amount is administered to the subject to treat the disorder.

Preferably the therapeutically effective amount is administered to the subject utilising a dosing regimen selected from the group consisting of: twice hourly; hourly; once every six hours; once every 8 hours; once every 12 hours; once daily; twice weekly; once weekly; once every 2 weeks; once every 6 weeks; once a month; every 2 months; every 3 months; once every 6 months; and once yearly.

Preferably the therapeutically effective amount is administered to the subject using a method selected from the group consisting of: orally, intravenously, intramuscularly, intrathecally, subcutaneously, sublingually, buccally, rectually, vaginally, topically, parentally, mucosally, by the ocular route, by the otic route, nasally, by inhalation, cutaneously, transdermally, and systemically.

In a further preferred embodiment, the disorder is caused by inflammation.

In a further preferred embodiment, the disorder is caused by neuro-inflammation.

Preferably, the disorder is a neurological disorder. More preferably, the neurological disorder is selected from the group consisting of: Alzheimer's disease; Parkinson's disease; multiple sclerosis; amyotrophic lateral sclerosis; cerebral ischemia; traumatic brain injury; rheumatoid arthritis; chronic migraine; epilepsy; autism spectrum disorder; attention deficit hyperactivity disorder; cerebral palsy and relevant subtypes; neuropathic pain; and depression.

In a further preferred embodiment, the ASD is ASD Level II/III and being either ‘Mildly ill’, ‘Moderately ill’, ‘Markedly ill’ or ‘Severely ill’ on the CGI Severity scale.

In a further preferred embodiment, the neurological disorder is a paediatric neurological disorder. For example, the paediatric neurological disorder is PANS. In one embodiment the paediatric neurological disorder is selected from the group consisting of: PANS/PANDAS, Tourette syndrome, cerebral palsy, ataxia, attention deficit hyperactivity disorder (ADHD), Lennox Gastaut, Dravet syndrome, leukodystrophy and subtypes, Reye syndrome, Rett syndrome, Fragile-X syndrome, Phelan-McDermid syndrome, Angelman syndrome, Pitt-Hopkins syndrome, Prader-Willi syndrome, a metabolic disorder and a glycosylation disorder.

In a preferred embodiment, the PANS is selected from the group consisting of: PANDAS and PITANDS.

In a preferred embodiment, the metabolic disorder is selected from the group consisting of: Hurler syndrome, Niemann-Pick disease, Tay-Sachs disease, Gaucher disease, Fabry disease and Krabbe disease, Galactosemia, Maple syrup urine disease, Phenylketonuria, glycogen storage disease, mitochondrial disorders, Friedreich ataxia, Peroxisomal disorder, metal metabolism disorder, organic acidemias and urea cycle disorders.

In a preferred embodiment, the glycosylation disorder is selected from the group consisting of: disorders of N-glycosylation, disorders of O-glycosylation, disorders of glycosylphosphatidylinositol (GPI) anchor and glycolipid anchor, and disorders of glycosylation pathways.

In a preferred embodiment, the paediatric neurological disorder is associated with neuroinflammation.

In a further preferred embodiment, the treatment reduces the neuro-inflammation. Preferably, the treatment suppresses the activity of any one of the following biomarkers: COX-2; iNOS; TNF-alpha; IL-2; IL-12 and GS-MCF.

A subject that can be treated with the invention will include humans as well as other mammals and animals.

In a further preferred embodiment, the method comprises administering to a patient in need thereof a therapeutically effective amount of the dosage form of the invention together with an additional active ingredient. In a preferred form, the additional active ingredient is administered using a dosing regimen selected from the group consisting of: at the same time as administering the dosing form of the invention; before administering the dosing form of the invention; after administering the dosing form of the invention; concurrently with administering the dosing form of the invention; sequentially before administering the dosing form of the invention; and sequentially after administering the dosing form of the invention.

Preferably, the additional active ingredient is selected from the group consisting of: a polypeptide; an antibody; a NSAID; a neuro-regulator; and a neurotransmitter, steroids—all relevant classes (corticosteroids), analgesics, anti-psychotics, anti-depressants, immuno-therapy.

In one preferred embodiment, the NSAID is selected from the group consisting of: aspirin, ibuprofen, naproxen, diclofenac, celecoxib, ketorolac, meloxicam, esomeprazole, naproxen, diclofenac, misoprostol, nabumetone, indomethacin, mefenamic acid, etodolac, piroxicam, ketoprofen, diflunisal, oxaprozin, flurbiprofen, sulindac, tolmetin, prednisolone and fenoprofen.

In one embodiment, the additional active ingredient is selected from the group consisting of: diclofenac, prednisone, celecoxib and psylocibin.

In a further preferred embodiment, the ratio of cannabinoid component and the additional active ingredient is selected from the group consisting of: 1 unit w/w of cannabinoid: 1 unit w/w/of the additional active ingredient; 2:1; 3:1; 4:1; 5:1; between 10,000:1 and 1:1; between 1,000:1 and 1:1; between 500:1 and 1:1; between 100:1 and 1:1; between 50:1 and 1:1; and between 10:1 and 1:1.

In a further preferred embodiment, the ratio of the additional active ingredient and cannabinoid is selected from the group consisting of: 1 unit w/w of the additional active ingredient and 1 unit w/w/of the cannabinoid; 2:1; 3:1; 4:1; 5:1; between 10,000:1 and 1:1; between 1,000:1 and 1:1; between 500:1 and 1:1; between 100:1 and 1:1; between 50:1 and 1:1; and between 10:1 and 1:1.

In a further preferred embodiment, the ratio of CBDA and the additional active ingredient is selected from the group consisting of: 1 unit w/w of CBDA: 1 unit w/w/of the additional active ingredient; 2:1; 3:1; 4:1; 5:1; between 10,000:1 and 1:1; between 1,000:1 and 1:1; between 500:1 and 1:1; between 100:1 and 1:1; between 50:1 and 1:1; and between 10:1 and 1:1.

In a further preferred embodiment, the ratio of the additional active ingredient and CBDA is selected from the group consisting of: 1 unit w/w of the additional active ingredient and 1 unit w/w/of the CBDA; 2:1; 3:1; 4:1; 5:1; between 10,000:1 and 1:1; between 1,000:1 and 1:1; between 500:1 and 1:1; between 100:1 and 1:1; between 50:1 and 1:1; and between 10:1 and 1:1.

In one preferred embodiment, the neuroregulator is a psychedelic substance.

Preferably, the neuroregulator is selected from the group consisting of: 3,4-methylenedioxymethamphetamine; lysergic acid diethylamide; and psilocybin.

The effect of the administered therapeutic composition can be monitored by standard diagnostic procedures.

### Use of a Composition in the Manufacture of a Medicament

Uses are within the scope of this invention. The invention also provides a use of the composition of the first aspect of the invention in the manufacture of a medicament for the treatment of a disorder.

In one preferred embodiment, the invention is the use of a composition comprising the following cannabinoids:
w/w %
CBDA 40-60%;
CBD 1-5%;
CBG 1-10%;
CBDP 1-5%;
CBDB 1-5%;
CBGA 1-10%;
CBN 1-3%
and
THC<1%;
in the manufacture of a medicament for the treatment of a paediatric neurological disorder.

In a further embodiment, the cannabinoids of the composition are present in amounts selected from the group consisting of:
Composition 1 comprising
w/w %
CBDA 50%;
CBD 2%;
CBG 5%;
CBDP 2%;
CBDB 2%;
CBGA 5%;
CBN 3% and
THC<0.3%;
and
Composition 2 comprising
w/w %
CBDA 45%;
CBD 1%;
CBG 4%;
CBDP 1%;
CBDB 2%;
CBGA 4%;
CBN 2% and
THC<0.2%;
in the manufacture of a medicament for the treatment of a paediatric neurological disorder.

In a further embodiment, the paediatric neurological disorder is PANS.

In a further embodiment, the PANS is selected from the group consisting of: PANDAS and PITAND.

In a further embodiment, the composition further comprises an oil selected from the group consisting of: a synthetic oil; plant based oil; mineral oil; canola oil; and olive oil.

In a further embodiment, the composition comprises less than 5% w/w terpenes.

In a further embodiment, the composition comprises less than 2% w/w organic plant material.

In a further embodiment, the composition comprises less than 2% w/w of plant phenols.

In a further embodiment, the cannabinoid component of the composition is selected from the group consisting of: between 1 and 500 mg/ml; between 10 and 100 mg/ml; be at a concentration of 50 mg/ml.

In a further embodiment, the CBDA component of the composition is selected from the group consisting of: between 1 and 500 mg/ml; between 10 and 100 mg/ml; be at a concentration of 50 mg/ml.

In a further embodiment, the composition has a UPLC mass chromatogram corresponding to FIG. 3 utilising the conditions described in Example 1.

### Processes

The invention also provides a process of extracting the composition of the first aspect of the invention from Cannabis plant material, said process comprising the steps of:
1) Grinding the Cannabis plant material to a sufficient grind size;
2) Contacting the grind produced by step a) with oil;
3) Mixing the grind and oil for a sufficient time period to form a mixture;
4) Pressing the mixture to reclaim the oil;
5) Centrifuging the oil to further refine the oil; and
6) Collecting the oil extract in a suitable container/steel vessel.

In a further preferred embodiment, the Cannabis plant material is derived from Cannabis sativa L.

In a further preferred embodiment, the sufficient grind size is selected from the group consisting of: between 0.1 mm and 3 mm; between 1 mm and 2 mm; and between 0.5 mm and 2.5 mm.

In a further preferred embodiment, the sufficient time period is selected from the group consisting of: between 30 minutes and 2 hours; between 45 minute and 1.5 hours; 1 hr.

In a further preferred embodiment, the ratio of grind material to oil at step (2) is selected from the group consisting of: 400 mg of grind: 1 ml of oil; 300 mg of grind: 1 ml of oil; 200 mg of grind: 1 ml of oil; 100 mg of grind: 1 ml of oil; and 333 mg of grind: 1 ml of oil.

Preferably, the oil is olive oil.

The invention also provides an alternative process of extracting the composition of the first aspect of the invention from Cannabis plant material, said process comprising the steps of:
1) Grinding the Cannabis plant material to a sufficient grind size;
2) Contacting the grind produced by step a) with an alcohol;
3) Mixing the grind and alcohol for a sufficient time period to form a mixture;
4) Sonicating the mixture;
5) Centrifuging the mixture; and
6) Collecting the alcohol extract in a suitable container.

In a further preferred embodiment, the alcohol is ethanol.

In a further preferred embodiment, the alcohol is selected from the group consisting of: ethanol, isopropyl alcohol, methyl alcohol, benzyl alcohol, 1,4-butanediol, 1,2,4-butanetriol, butanol, 1-butanol, 2-butanol, and tert-butyl alcohol.

In a further preferred embodiment, the sufficient grind size is selected from the group consisting of: between 0.1 mm and 3 mm; between 1 mm and 2 mm; and between 0.5 mm and 2.5 mm. In a further preferred embodiment, the sufficient time period is selected from the group consisting of: between 30 minutes and 2 hours; between 45 minute and 1.5 hours; 1 hr.

In a further preferred embodiment, the ratio of grind material to alcohol at step (2) is selected from the group consisting of: 400 mg of grind: 1 ml of alcohol; 300 mg of grind: 1 ml of alcohol; 200 mg of grind: 1 ml of alcohol; 100 mg of grind: 1 ml of alcohol; 100 mg of grind: 4 ml of alcohol; 100 mg of grind: 3 ml of alcohol; 100 mg of grind: 2 ml of alcohol; and 333 mg of grind: 1 ml of alcohol.

### Product of the Process

The invention also provides a product produced from the process described above.

### Kit

The invention also provides a kit comprising the dosage form of one aspect of the invention together with instructions for its use.

### Device

Devices are within the scope of the invention. In a preferred embodiment, the invention provides a device, wherein the device comprises: (1) the composition as described in the first aspect of this invention; and (2) an applicator.

### Method for Stabilising

Methods for stabilizing the composition are within the scope of the invention.

In a further preferred embodiment, the said method protects the composition against degradation.

In yet a further preferred embodiment, the composition retains its effective biological activity for a period selected from the group consisting of; greater than 24 hours; greater than 36 hours; greater than 48 hours.

The addition of approved pharmaceutical excipients to stabilise the composition is preferred from a safety standpoint, as the simpler methodology is likely to produce a less variable outcome and the choice of excipient can be limited to those with Generally Regarded as Safe (GRAS) status. Excipients for the stabilisation of protein solutions can be classified into four broad categories: salts, sugars, polymers or protein/amino acids, based on their chemical properties and mechanism of action. Salts (e.g., chlorides, nitrates) stabilise the tertiary structure of proteins by shielding charges through ionic interactions. Sugars (e.g., glycerol, sorbitol, fructose, trehalose) increase the surface tension and viscosity of the solution to prevent protein aggregation. Similarly, polymers (e.g. polyethylene glycol, cellulose derivatives) stabilise the protein tertiary structure by increasing the viscosity of the solution to prevent protein aggregation and intra- and inter-molecular electrostatic interactions between amino acids in the protein. Proteins (e.g. human serum albumin) are able to stabilise the structure of other proteins through ionic, electrostatic and hydrophobic interactions. Similarly, small amino acids with no net charge, such as alanine and glycine, stabilise proteins through the formation of weak electrostatic interactions.

As discussed above, the medicaments of the present invention may include one or more pharmaceutically acceptable carriers. The use of such media and agents for the manufacture of medicaments is well known in the art. Except insofar as any conventional media or agent is incompatible with the pharmaceutically acceptable material, use thereof in the manufacture of a pharmaceutical composition according to the invention is contemplated. Pharmaceutical acceptable carriers according to the invention may include one or more of the following examples:
a. surfactants and polymers, including, however not limited to polyethylene glycol (PEG), polyvinylpyrrolidone, polyvinylalcohol, crospovidone, polyvinylpyrrolidone-polyvinylacrylate copolymer, cellulose derivatives, HPMC, hydroxypropyl cellulose, carboxymethylethyl cellulose, hydroxypropylmethyl cellulose phthalate, polyacrylates and polymethacrylates, urea, sugars, polyols, and their polymers, emulsifiers, sugar gum, starch, organic acids and their salts, vinyl pyrrolidone and vinyl acetate; and/or
b. binding agents such as various celluloses and cross-linked polyvinylpyrrolidone, microcrystalline cellulose; and/or (3) filling agents such as lactose monohydrate, lactose anhydrous, microcrystalline cellulose and various starches; and/or
c. filling agents such as lactose monohydrate, lactose anhydrous, mannitol, microcrystalline cellulose and various starches; and/or
d. lubricating agents such as agents that act on the increased ability of the dosage form to be ejected from the packaging cavity, and/or
e. sweeteners such as any natural or artificial sweetener including sucrose, xylitol, sodium saccharin, cyclamate, aspartame, and acesulfame K; and/or
f. flavouring agents; and/or
g. preservatives such as potassium sorbate, methylparaben, propylparaben, benzoic acid and its salts, other esters of parahydroxybenzoic acid such as butylparaben, alcohols such as ethyl or benzyl alcohol, phenolic chemicals such as phenol, or quarternary compounds such as benzalkonium chloride; and/or
h. buffers; and/or
i. diluents such as pharmaceutically acceptable inert fillers, such as microcrystalline cellulose, lactose, dibasic calcium phosphate, saccharides, and/or mixtures of any of the foregoing; and/or
j. absorption enhancer such as glyceryl trinitrate; and/or
k. other pharmaceutically acceptable excipients.

Medicaments of the invention suitable for use in animals and in particular in human beings typically must be sterile and stable under the conditions of manufacture and storage.

The invention also provides a composition, methods and processes as described by the foregoing examples.

The present invention will now be described with reference to the following non-limiting Examples. The description of the Examples is in no way limiting on the preceding paragraphs of this specification, however, is provided for exemplification of the methods and compositions of the invention.

### EXAMPLES

It will be apparent to persons skilled in the milling and pharmaceutical arts that numerous enhancements and modifications can be made to the above-described processes without departing from the basic inventive concepts. For example, in some applications the biologically active material may be pretreated and supplied to the process in the pretreated form. All such modifications and enhancements are considered to be within the scope of the present invention, the nature of which is to be determined from the foregoing description and the appended claims. Furthermore, the following Examples are provided for illustrative purposes only, and are not intended to limit the scope of the processes or compositions of the invention.

### A Example 1-Extraction and Purification of NTI164

### A.1 Study Aim

To extract and identify the most desirable components from the NTI164 plant strain using an inert oil-based extraction process.

### A.2 Materials and Methods

### A.2.1 NTI164 Plant Material

The NTI164 plant is a full-spectrum medicinal Cannabis plant (genus species Cannabis sativa) which the inventors subsequently identified to contain cannabidiolic acid (CBDA), cannabidiol (CBD), cannabigerolic acid (CBGA), cannabidivarin (CBDV) and cannabinol (CBN) but which has >0.03% tetrahydrocannabinol (THC). The NTI164 plant was cultivated, dried and packaged under an Office of Drug Control (ODC) license and permit as per Good Manufacturing Processes (GMP) and TGO 93 and 100 guidelines.

### A.2.2 Extraction Method-Oil Based

Equipment: The following equipment was used: 10 mL glass scintillation bottles with lids; Cobram's Estate olive oil; plant grinder (similar to a coffee or food grade grinder) pore size up to 50 μM; Whatman paper, grade 1; pipettes; weight scale (transfer boats and spoons); Eppendorf tubes; 50 mL falcon tubes; bench top centrifuge (Eppendorf Centrifuge 5702); Oz Design Brand 6 Litre Fruit, Wine and Cider Press.

Extraction: Pressing and Centrifugation: All work is undertaken at standard lab temperatures (18-22 °C). The buds of NTI164 were stripped off hard stalks and the stalks discarded. The grinder was cleaned with 70% EtOH and the grinding compartment was filled with dried plant material. The material was ground on the finest of the three setting for 10 seconds (1-2 mm particle size). The grounds were then mixed with 100 ml of olive oil in an autoclaved Schott bottle at a ratio plant/oil of 333 mg/mL. It was then placed on a stirrer at room temperature for 1 hour, stirred with magnetic flea (50 rpm). The oil plus plant mixture is then put into the Oz Design Brand 6 Litre Fruit, Wine and Cider Press to reclaim the oil component from the plant (the mash). The reclaimed oil was then placed into 50 mL falcon tubes and spun at 300 g for 15 minutes at room temperature (Isolation 1). The oil was then removed into a clean Schott bottle and keeping track of the volume reclaimed. The recovery of the oil for Isolation 1 is approximately 40%. The mash is discarded following each isolation. To the reclaimed oil, we added a further 333 mg/mL ground plant/oil (a further 100 ml) material and repeated the 1 hour mix, and reclaimed and re-used oil, until a total of 999 μg/mL (3×100 ml) of plant/oil mixture passed through (Isolation 2). The recovery of the oil for Isolation 2 is approximately 50%. For the final time, we placed into falcon tubes and spin as discussed above (Isolation 3). The recovery of the oil at for Isolation 3 is approximately 50%. The oil only was then collected and placed the oil into Eppendorf tubes for processing. This triplicate extraction method resulted in a total volume of 50 ml of final product at a concentration of 48 mg of CBDA to 1 ml of olive oil determined using UPLC potency testing using the methods described below.

### A.2.3 Extraction Method-Ethanol Based

Extraction: Pressing and Centrifugation: An alternative method includes an extraction based on the use of ethanol. In this method, 500 milligrams of ground plant material of NTI164 is mixed with 20 ml of ethanol in a 50 ml centrifuge tube. The tubes are shaken vigorously for 60 seconds and then placed into a sonicate bath at 30 C for 10 minutes. Samples are then placed on a shaker (200 rpm) for 30 minutes. Once completed, placed in a centrifuge, and centrifuged at 4400 rcf for 5 minutes. The supernatant can then be assessed in the various preclinical models.

### A.2.4 Analytical Analysis

Ultra-performance liquid chromatography (UPLC) reverse-phase and liquid chromatography mass spec (LCMS) were used to identify the components in the NTI164 concentrate derived from the methods discussed above. The analysis was performed using an integrated (U) HPLC system and a single quadrupole mass spectrometer detector with electrospray ionization (ESI) interface.

The UPLC settings and conditions used were: Cortecs UPLC Shield RP 18, (0 A 1.6 uM, 2.1×100 mm); Analytical flow rate: 0.7 ml/min; Mobile phase A: Water 0.1% TFA; Mobile phase B: Acetonitrile; Isocratic: 41:59 mobile phase A/mobile phase B; Temp: 35 C; Detector: Acquity UPLC PDA; Injection volume: 0.7 μL for 1.0 mg/ml reference standard preparations, sample solutions scaled appropriately; Software: Empower 3CDS. Reference standard solutions were obtained from Novachem, Cerilliant Corporation (TX, USA). These were pre-dissolved solutions all previously shown to be suitable for the generation of calibration curve.

A mixture of 16 cannabinoids in methanol was prepared, containing 10 ppm each of cannabidivarin (CBDV), cannabidiol (CBD), cannabigerol (CBG), tetrahydrocannabivarin (THCV), cannabinol (CBN), Δ⁹-tetrahydrocannabinol (Δ⁹-THC), Δ⁸-tetrahydrocannabinol (Δ⁸ THC), cannabichromene (CBC), their respective acidic forms and cannabicyclol (CBL). All solvents used were LCMS grade, and standards were prepared by diluting with 90% mobile phase B and 10% deionized water. Detailed analytical conditions for the UPLC-LCMS analysis are listed in Table 1.

TABLE 1
The parameters and conditions for UPLC and LCMS analysis
HPLC

| | |
|---|---|
| Column + Guard column | Nexteaf CBX 11, 1.8 μm, 3.0 × 100 mm |
| Mobile phase A | 0.1% formic acid and 5 mM ammonium |
| | formate in H₂O |
| Mobile phase 3 | 0.1% formic acid in methanol/acetonitrile |
| | (50:50 v/v) |
| Gradient | 83% mobile phase B to 98% mobile |
| | phase B in 6.5 min |
| Flow rate | 0.5 ml/min |
| Oven temperature | 30 °C |
| Injection volume | 5 μL |

LCMS

| | |
|---|---|
| Ionization | ESI |
| Interface temperature | 350 °C |
| DL temperature | 250 °C |
| Nebulizing gas flow | 15 L/min |
| Heat block | 400 °C |
| Drying gas flow | 1.5 L/min |
| Q-array DC voltage | 55 V |

### A.3 Results

### A.3.1 UPLC and Lcms Analytical Results

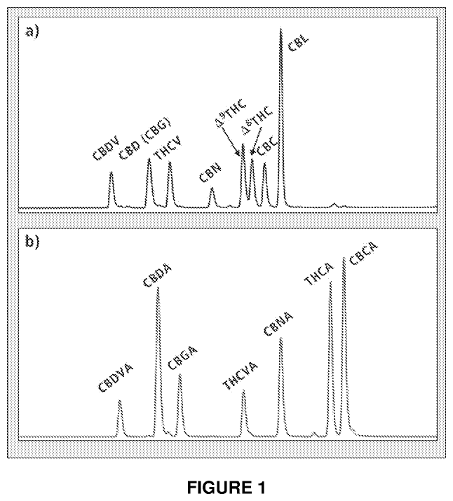

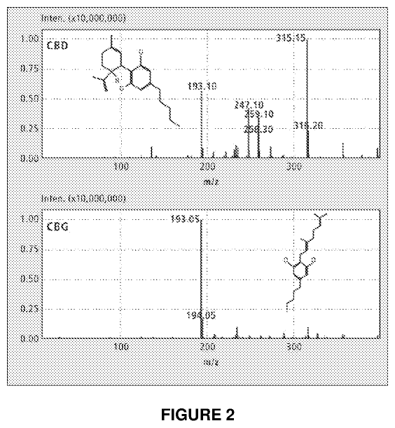

FIG. 1 shows the separation of the cannabinoids in a mixed standard solution (that is a reference solution). Under the conditions of the experiment, neutral cannabinoids such as 49-THC, CBD and CBL ionize in positive mode, while their respective acidic forms ionize in negative mode. Although CBD and CBG coelute from the column, their molecular weights differ, and they can be identified by mass spectra. In addition, FIG. 2 shows a difference between the SID fragmentation patterns obtained for CBD and CBG (that is; as further reference solution). These highly specific results show the advantage of LCMS over LC-UV for analysis and identification of cannabinoids.

FIG. 3 presents the UPLC mass chromatogram for NTI164 extracted using the oil-based method. These results found that the NTI164 extract (oil suspension) contained the following components presented in Table 2. Additional components will include flavonoids, proteins, phenols, sterols and esters. These are known components that make up 30-40% of the full plant Cannabis material. Table 4 presents the accompanying exlution times for the UPLC mass chromatograpm for FIG. 3 and area under the peaks for the CBD peaks identified.

TABLE 2
Components in NTI164 oil extracted (at two decimal places and
rounded up beyond 0.5, and rounded down below 0.5)

| Component | w/w % | w/w % |
|---|---|---|
| Cannabidiol acid (CBD-A) | 48.78% | 49% |
| Cannabidiol (CBD) | 1.89%; | 2%; |
| Cannabigerol (CBG) | 4.88%; | 5%; |
| Cannabidiphorol (CBDP) | 1.68%; | 2%; |
| Canabidibutol (CBDB) | 1.76%; | 2%; |
| Cannabigerolic acid (CBGA) | 4.76%; | 5%; |
| Tetrahydrocannabinol (THC) | <0.18% | <0.3% |
| Terpenes | <5% | <5% |
| Organic plant material - including phenols | 2% | 2% |

Table 3 presents the NTI164 composition extracted using the ethanol extraction and the components quantified using the methods herein described.

TABLE 3
Components in NTI164 ethanol extracted (at two decimal places
and rounded up beyond 0.5, and rounded down below 0.5)

| Component | w/w % | w/w % |
|---|---|---|
| Cannabidiol acid (CBD-A) | 45.28% | 45% |
| Cannabidiol (CBD) | 1.39%; | 1%; |
| Cannabigerol (CBG) | 3.88%; | 4%; |
| Cannabidiphorol (CBDP) | 1.18%; | 1%; |
| Canabidibutol (CBDB) | 1.56%; | 2%; |
| Cannabigerolic acid (CBGA) | 3.76%; | 4%; |
| Tetrahydrocannabinol (THC) | <0.18% | <0.2% |
| Terpenes | <5% | <5% |
| Organic plant material - including phenols | 2% | 2% |

Table 4 presents the accompanying elution times for the UPLC mass chromatograpm for FIG. 3 (NTI164) and area under the peaks for the CBD peaks identified.

TABLE 4
elution times and area under the peak

| | | Elution time | Area under the |
|---|---|---|---|
| Component | w/w % | (min) | peak (quantity) |
| Cannabidiol (CBD) | 1.89%; | 3.122 | 20876 |
| Cannabidiol acid (CBD-A) | 48.78% | 3.456 | 1161809 |
| Cannabigerol (CBG) | 4.88%; | 3.673 | 16332 |
| Cannabidiphorol (CBDP) | 1.68%; | See | See comments |
| | | comments | |
| Canabidibutol (CBDB) | 1.76%; | See | See comments |
| | | comments | |
| Cannabigerolic acid (CBGA) | 4.76%; | 5.121 | 13492 |
| Tetrahydrocannabinol (THC) | <0.18% | 5.437 | 1980 |
| THCA | <0.18% | 8.617 | 30141 |
| Terpenes | <5% | | |
| Organic plant material - | 2% | | |
| including phenols | | | |

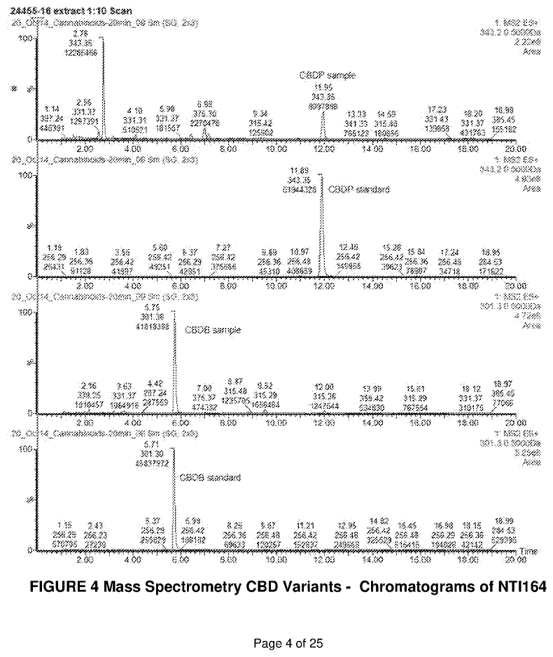

Please note that the rarer cannabinoids such as CBDB and CBDP are only detected using Quad MS (which is different to the routine HPLC used for the other cannabinoids). These results are presented in FIG. 4.

### B Example 2-Characterisation of the Stability Properties of NTI164

B.1 Study Aim

To assess the stability of the NTI164 samples suspended in oil formulation at room temperature.

### B.2 Materials and Methods

### B.2.1 Sample Preparation

Triplicate samples of NTI164 were prepared using the methods described above.

For control samples, three representative pre-prepared concentrate samples NTI164 (oil and dried flower) were obtained as follows. Oil S=samples were prepared as outlined above: For flower, a portion of homogenized plant material was added to acetonitrile or ethanol and sonicated for 20 minutes. The subsequent extract was filtered through a 0.22 μm syringe tip filter directly into a 2 mL sample vial for analysis. Concentrates were prepared similarly with isopropanol as the extraction solvent.

### B.2.2 Sampling

Samples of NTI164 were assayed on a weekly basis and CBDA was used as a main marker/stability indicator. The ACQUITY UPLC H-Class System combined with the CORTECS UPLC Shield RP18 particle chemistry was used to provide a UPLC isocratic separation of main cannabinoids in a 10.5-minute cycle time. Analytical methods using UPLC were used as described above.

Reference standard solutions were obtained from Cerilliant Corporation (Round Rock, TX). These pre-dissolved solutions have been previously shown to be suitable for the generation of calibration curves.

Preparation of standard curves were performed as follows. Linearity of primary cannabinoids (−)_{Δ}⁹-THC and CBD were determined for 10 concentrations between 0.004 mg/mL and 1.000 mg/mL, prepared via serial dilution in methanol using appropriate standards as a representative demonstration of method linearity. Table 5 outlines the cannabinoids used in the separation.

TABLE 5
The cannabinoids used in the separation

| | | Cannabinoid | CAS |
|---|---|---|---|
| No. | Cannabinoid | abbreviation | number |
| 1 | Delta-9-tetrahydrocannabinol | (-)Δ9-THC | 1972-08-03 |
| 2 | Delta-9-tetrahydrocannabinolic acid | THC-A | 23978-85-0 |
| 3 | Cannabidiol | CBD | 13956-29-1 |
| 4 | Cannabidiol acid | CBD-A | 1244-58-2 |
| 5 | Cannabinol | CBN | 521-35-7 |
| 6 | Delta-8-tetrahydrocannabinol | (-)Δ8-THC | 5957-75-5 |
| 7 | Cannabigerol | CBG | 25654-31-3 |
| 8 | Cannabigerolic acid | CBG-A | 25555-57-1 |
| 9 | Cannabichromene | CBC | 20675-51-8 |
| 10 | Cannabichromenic acid | CBC-A | 185505-15-1 |
| 11 | Tetrahydrocannabivarin | THCV | 31262-37-0 |
| 12 | Tetrahydrocannabivarinic acid | THCV-A | 39986-26-0 |
| 13 | Cannabidivarin | CBDV | 24274-48-4 |
| 14 | Cannabidivarinic acid | CBDV-A | 31932-13-5 |
| 15 | Cannabicyclol | CBL | 21366-63-2 |
| 16 | Cannabicyclolic acid | CBL-A | 40524-99-0 |

### B.3 Results

### B.3.1 UPLC Analytical Results

The ACQUITY UPLC H-Class System combined with the CORTECS UPLC Shield RP18 particle chemistry was used to provide a UPLC isocratic separation of main cannabinoids in a 10.5-minute cycle time. Samples of NTI164 were assayed on a weekly basis and CBDA was used as a main marker as a stability indicator. Results presented in Table 6 demonstrate that NTI164 is stable at room temperature within an inert oil media over 6 weeks. There is no decarboxylation or product degradation observed over this time frame.

TABLE 6
Stability of NTI164 at room temperature

| Time Point | | | Day 0 | Day 2 | Day 7 | Day 14 | Day 28 | Week 5 | Week 6 | Week 32 |
|---|---|---|---|---|---|---|---|---|---|---|
| Testing Date | | | Jul. 13, | Jul. 17, | Jul. 20, | Jul. 29, | Aug. 12, | Aug. 19, | Aug. 27, | Sep. 12, |
| | | | 2021 | 2021 | 2021 | 2021 | 2021 | 2021 | 2021 | 2022 |
| SAMPLE ID | | | 28206-7 | 28206-7 | 28206-7 | 28206-7 | 28206-7 | 28206-7 | 28206-7 | 28206-7 |
| CANNABINOIDS | Cas No's | Units | | | | | | | | |
| Cannabidiol (CBD) | 13956-29-1 | mg/mL | 0.46 | 0.56 | 0.53 | 0.56 | 0.54 | 0.60 | 0.51 | 0.48 |
| Cannabidiol acid | 1244-58-2 | mg/mL | 48.16 | 48.19 | 48.62 | 48.09 | 47.12 | 48.35 | 47.56 | 47.01 |
| (CBD-A) | | | | | | | | | | |
| Tetrahydrocannabinol | 1972-08-03 | mg/mL | <0.1 | <0.1 | <0.1 | <0.1 | <0.1 | 0.14 | <0.1 | 0.11 |
| (D9-THC) | | | | | | | | | | |
| (Tetrahydrocannabinolic | 23978-85-0 | mg/mL | 0.66 | 0.72 | 0.64 | 0.69 | 0.36 | 0.79 | 0.73 | <0.1 |
| acid) (D9-THCA) | | | | | | | | | | |
| Cannabidivarin | 24274-48-4 | mg/mL | <0.1 | <0.1 | <0.1 | <0.1 | <0.1 | 0.10 | <0.1 | 0.13 |
| (CBDV) | | | | | | | | | | |
| Cannabigerol | 25555-57-1 | mg/mL | 1.07 | 1.18 | 1.20 | 1.19 | 0.81 | 1.08 | 1.00 | 1.05 |
| Acid(CBG-A) | | | | | | | | | | |
| Cannabigerol (CBG) | 25654-31-3 | mg/mL | 5.21 | 5.23 | 5.14 | 5.1 | 5.1 | 5.30 | 5.28 | 5.12 |
| Tetrahydrocannabivarin | 28172-17-0 | mg/mL | <0.1 | <0.1 | <0.1 | <0.1 | <0.1 | <0.1 | <0.1 | <0.1 |
| (THCV) | | | | | | | | | | |
| Cannabinol (CBN) | 521-35-7 | mg/mL | 2.32 | 2.54 | 2.48 | 2.33 | 2.56 | 2.22 | 2.27 | 2.21 |
| DELTA-8-THC | 5957-75-5 | mg/mL | <0.1 | <0.1 | <0.1 | <0.1 | <0.1 | <0.1 | <0.1 | <0.1 |
| (D8-THC) | | | | | | | | | | |
| Cannabichrome(CBC) | 20675-51-8 | mg/mL | 1.23 | 1.19 | 1.34 | 1.22 | 1.27 | 1.20 | 1.15 | 1.22 |

### C Example 3-Characterisation of the Biological Properties of NTI164

C.1 Study Aim

To assess the anti-inflammatory and neuro-protective effects of NTI164 in neuronal and microglial cell lines.

Neuroinflammation is one of the main triggers of neurodegeneration. Research into the factors and pathways able to induce the first steps of the inflammatory response would lead to the identification of potential therapeutic targets through which to halt the progression of many disorders.

### C.2 Materials and Methods

### C.2.1 Sample Preparation and Dilutions

500 mg of dried plant material of NTI164 is suspended in 20 ml of absolute ethanol (using 50 ml blue top falcon tubes appropriate for centrifugation) and stirred/shaken vigorously for 60 seconds. The tubes are then placed in a sonication bath for 10 mins at 35-40 C. Upon completion of the sonication samples are then placed in a tray shaker (200 rpm) for 30 minutes at room temperature. Once completed the samples are then centrifuged at 4400 rpm for 5 minutes. The supernatant is collected for testing and development.

Units used to describe treatments for test products and concentrations for NTI164.
a. 1/1000 dilution of extract—10 UL (stock material is NTI164—10 UL, which equates to 2 μg/ml of CBDA)
b. 1/3000 dilution of extract—3 UL (stock material is NTI164—3 UL, which equates to 6 μg/ml of CBDA)
c. 1/10000 dilution of extract—1 UL (stock material is NTI164—1 UL, which equates to 0.1 μg/ml of CBDA).

For the CBD sample, a pure standard (in powder form) was used. CBD 98% isolate was purchased as a reference standard from LGC Standards (London UK) (CAS No. 13956-29-1). The CBD standard reference was prepared at concentration of 1 mg/ml (in acetonitrile). CBD dilutions were made in acetonitrile as follows: 2 μg/ml; 6 μg/ml; and 0.1 μg/ml.

The final concentration of NTI164 (CBDA equivalent) and CBD that was used in these studies was 2 μg/ml.

### C.2.2 Microglial BV2 Culture

The immortalized microglia cell line, BV2, was purchased from the American Tissue Culture Collection. BV2 were cultured in RPMI media containing gentamycin and supplemented with 10% FBS for expansion and 5% fetal bovine serum (FBS) when plated for experiments. All cells were from between passage numbers 39 and 45. Cells were plated at 45,000 cells/mm2 and treated 24 hours after plating with phosphate buffered saline (PBS, as a control) or interleurkin-1B+interferon-γ (IL-1B+IFNγ, to induce inflammation). To test the effects of NTI164 to alter the inflammatory response, NTI164 was applied one hour before inflammation (pre-treat) or one hour after (post-treat). NTI164 was applied at 10 μL, 3 μL or 1 μL from isolated obtained using the original extraction protocol: range determined from mass spec data=1.0-0.1 ug CBDA.

### C.2.3 Multiplex Cytokine/Chemokine Assay

Microglia media were harvested following treatment initiation was centrifuged briefly to remove particulates (300 g for 10 min). Cytokine and chemokine levels in the microglial media were measured using a Bio-Plex 200 with a 96-well magnetic plate assay according to the manufacturer's instructions (Bio-Rad). Cytokines and chemokines measured included IL-1α, IL-1β, IL-2, IL-6, IL-10, IL-12 (p70), IL-13, IL-17, G-CSF, GM-CSF, IFNγ, TNFα, CXCL1 (KC), CCL2 (MCP-1), and CCL5 (RANTES). All samples were run in duplicate and data were analyzed with the Bio-Plex Manager software.

### C.2.4 Immunohistochemistry (Protein Level) Assay

Cells were fixed for 10 min with 4% paraformaldehyde (PFA) in PBS. After 3×5 minutes washing with PBS, cells were incubated with primary antibodies (anti-COX2, anti-ARG1) 1:1000 overnight at 4 °C, and after 3×5 minutes washing in PBS, cells were then incubated in appropriate fluorescent secondary antibody 1:250 (Invitrogen) for 2 hours at room temperature. After a final wash, as previous, cells nuclei were stained with DAPI in the mounting media. Photomicrographs were taken of the cells in three fields of view per well from duplicate wells and analysed using Fiji for area coverage of each marker.

### C.2.5 Cell Viability (Mitochondrial Activity) Assay

Microglial viability was quantified using MTT [3-(4,5-dimethylthiazol-2-yl-)-2,5-diphenyl-2H-tetrazolium bromide; Sigma]. In this assay MTT, a tetrazolium dye, is bioreduced by the mitochondria into a formazan product that is insoluble in tissue culture medium. In brief, MTT was added to a final concentration of 250 μg/ml to cells at various time points following treatment with PBS, LPS or IL-4 with or without test product. After 30 min, formazan was dissolved in DMSO and the absorbance was measured at 490 nm using a spectrophotometer (Glomax Multi+; Promega, UK).

### C.2.6 Statistics

Data for replicates within experiments were averaged and then data from at least three independent experiments was analysed using Graph Pad Prism or students-t-test.

### C.3 Results

### C.3.1 INOS Expression

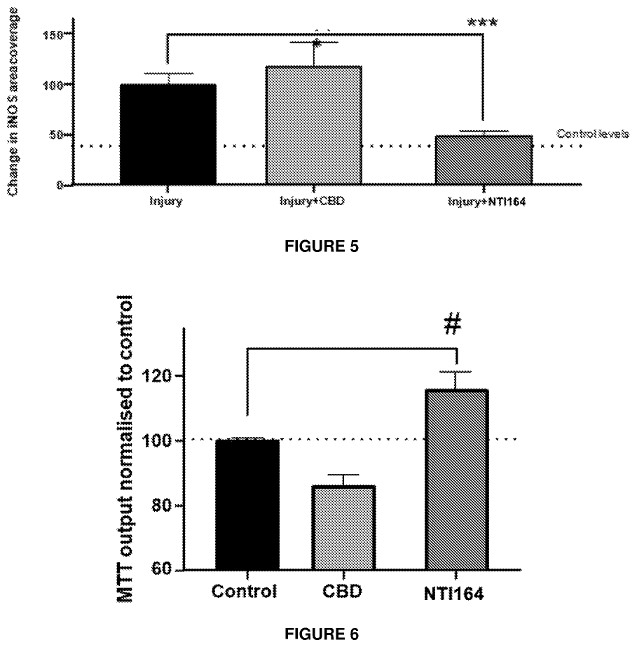

NTI164 normalised inflammation induced iNOS expression. iNOS expression is increased by inflammation and in inflammatory activated microglial cells, NTI164 normalized expression towards control levels, and therefore reduced the inflammatory process triggered by iNOS. Inducible nitric oxide synthase (iNOS) is one of three key enzymes generating nitric oxide (NO) from the amino acid L-arginine. Inducible nitric oxide synthase (iNOS) plays a critical role in the regulation of multiple sclerosis (MS) and experimental autoimmune encephalomyelitis (EAE). Previous studies have shown that iNOS plays pathogenic as well as regulatory roles in MS and EAE and many other neuro-inflammatory disorders. FIG. 5 demonstrates that NTI164 normalised inflammation induced iNOS expression.

### C.3.2 Neuronal Viability

NTI164 increased the number of viable neurons under basal conditions (short term exposure). Neuronal viability was quantified using MTT [3-(4,5-dimethylthiazol-2-yl-)-2,5-diphenyl-2H-tetrazolium bromide; Sigma]. NTI164 treated celled were able to increase the number of “healthy” cells under basal conditions following short term glutamate exposure. Cellular excitotoxicity was achieved via glutamate activation (3 mM). NTI164 was able to stimulate cell growth after short term glutamate induced “insult”.

The Cell Viability (Mitochondrial Activity) Assay (or MTT assay) was used to determine the cellular viability or metabolic activity in microcapsules within the cells. It is based on the ability of metabolically active cells to transform a water-soluble dye [3-(4,5-dimethylthiazol-2-yl)-2,5-diphenyltetrazolium bromide] into an insoluble formazan. Cell viability is a measure of the proportion of live, healthy cells within a population and cell viability assays are used to determine the overall health of cells. FIG. 6 presents the neuronal viability quantified using MTT [3-(4,5-dimethylthiazol-2-yl-)-2,5-diphenyl-2H-tetrazolium bromide].

### C.3.3 Maturation of Immature Neurons

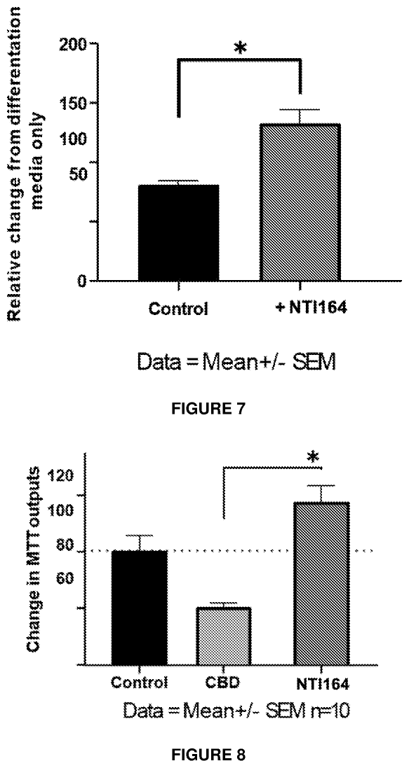

NTI164 stimulated the maturation of immature neurons into healthy cells even without the presence of any Glutamate induced insult. This study demonstrates that NTI164 can stimulate “healthy maturation” of immature neurons. This is process that may be vital after trauma, or damage. NTI164 is able to provide healthy neuronal ell development which is a vital process in recovery from neuro-inflammation, neuronal damage. FIG. 7 demonstrates that NTI164 stimulates the maturation of immature neurons into healthy cells even without the presence of any glutamate induced insult.

### C.3.4 Cell Death

NTI164 does not increase cell death in an excitotoxic cell injury paradigm.

FIG. 7 demonstrates that CBD is toxic in this paradigm while NTI164 is non-toxic and has positive effects on cell number and cell viability.

### C.3.5 ARG1 Expression

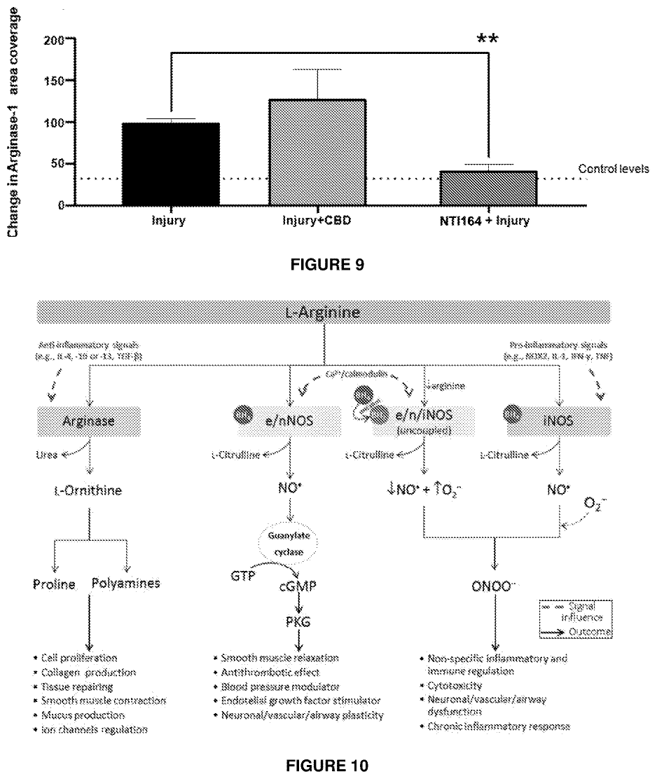

NTI164 normalises inflammation-induced (injured cells) Arg 1 expression. Macrophage specific upregulation of Arginase-1 is commonly believed to promote inflammation. Arginase 1 expression is increased by inflammation yet in inflammatory activated cells, NTI164 normalizes expression towards control levels. FIG. 9 shows the microglial responses under inflammatory conditions assessing Arginase 1 expressions.

FIG. 10 outlines the arginine metabolism and the effects it has on the overall balance of anti-inflammatory and pro-inflammatory signals.

### D Example 4-Preclinical Studies Relating to Biomarkers Involved in Neuroinflammation

D.1 Study Aim

To determine the effect of NTI164 on the levels of COX-2, IL2 and TNF-alpha in human derived microglial cells.

Many neurological disorders occur due to inflammation which is induced by dysregulation of immune responses.

For example, in Multiple Sclerosis (MS) which is a progressive inflammatory disease characterised by the loss of myelin sheath within the central nervous system. Typical symptoms include fatigue, walking difficulties and impaired speech and vision. Cyclooxygenase-2 (COX-2) is considered the main enzyme responsible for causing inflammation, the common mechanism of disease involved in MS. COX-2 is a powerful clinical biomarker in the assessment of disease progression and overall therapeutic management.

IL-2 plays an important role in immune regulation and an important role in MS progression. IL-12 is a cytokine that plays a key role in the pathogenesis of Multiple Sclerosis. Blocking this cytokine via a neutralizing antibody causes dramatic improvements in animal models of the disease, and multiple human trials.

TNF-alpha plays an important role plays an important role in dysregulation of acute inflammation involved in MS onset.

### D.2 Materials and Methods

### D.2.1 COX-2

Immunohistochemistry (Protein Level) Assay: Cells were fixed for 10 min with 4% paraformaldehyde (PFA) in PBS. After 3×5 minutes washing with PBS, cells were incubated with primary antibodies (anti-COX2) 1:1000 overnight at 4 °C, and after 3×5 minutes washing in PBS, cells were then incubated in appropriate fluorescent secondary antibody 1:250 (Invitrogen) for 2 hours at room temperature. After a final wash, as previous, cells nuclei were sained with DAPI in the mounting media. Photomicrographs were taken of the cells in three fields of view per well from duplicate wells and analysed using Fiji for area coverage of each marker.

### D.2.2 IL-2 AND TNF-Alpha

Multiplex Cytokine/Chemokine Assay: Microglia media harvested following treatment initiation was centrifuged briefly to remove particulates (300 g for 10 min). Cytokine and chemokine levels in the microglial media were measured using a Bio-Plex 200 with a 96-well magnetic plate assay according to the manufacturer's instructions (Bio-Rad). Cytokines and chemokines measured included IL-1α, IL-1β, IL-2, IL-6, IL-10, IL-12 (p70), IL-13, G-CSF, GM-CSF, IFNγ, TNFα, CXCL1 (KC), CCL2 (MCP-1), and CCL5 (RANTES). All samples were run in duplicate and data were analyzed with the Bio-Plex Manager software.

### D.2.3 Sample Preparation and Dilutions

500 mg of dried plant material of NTI164 is suspended in 20 ml of absolute ethanol (using 50 ml blue top falcon tubes appropriate for centrifugation) and stirred/shaken vigorously for 60 seconds. The tubes are then placed in a sonication bath for 10 mins at 35-40 C. Upon completion of the sonication samples are then placed in a tray shaker (200 rpm) for 30 minutes at room temperature. Once completed the samples are then centrifuged at 4400 rpm for 5 minutes. The supernatant is collected for testing and development.

Units used to describe treatments for test products and concentrations for NTI164.
a. 1/1000 dilution of extract—10 UL (stock material is NTI164—10 UL, which equates to 2 μg/ml of CBDA)
b. 1/3000 dilution of extract—3 UL (stock material is NTI164—3 UL, which equates to 6 μg/ml of CBDA)
c. 1/10000 dilution of extract—1 UL (stock material is NTI164—1 UL, which equates to 0.1 μg/ml of CBDA)

For the CBD sample, a pure standard (in powder form) was used. CBD 98% isolate was purchased as a reference standard from LGC Standards (London UK) (CAS No. 13956-29-1). The CBD standard reference was prepared at concentration of 1 mg/ml (in acetonitrile). CBD dilutions were made in acetonitrile as follows: 2 μg/ml; 6 μg/ml; and 0.1 μg/ml.

The final concentration of NTI164 (CBDA equivalent) and CBD that was used in these studies was 2 μg/ml.

### D.3 Results

### D.3.1 COX-2

The preclinical studies conducted in cells using immunohistochemistry analysis demonstrated that NTI164 can suppress and inhibit the expression of COX-2 in human derived microglial cells. When compared to CBD alone, NTI164 was up to three times more powerful in suppressing COX-2 both pre and post inflammatory insult. Refer to Table 7 below.

TABLE 7
Outlines the COX-2 suppression in cells when treated with NTI164
versus CBD alone.

| | | | | | |
|---|---|---|---|---|---|
| | | | | | Positive control |
| | | | | Positive control vs | vs CBD alone |
| N | Control Avg | NTI164 | CBD Avg | NTI164 treatment | treatment |

Pre-Inflammatory Exposure (exposure 1 hour prior to inflammatory insuff)

| | | | | | |
|---|---|---|---|---|---|
| 9 | 94.47 +/− 5.90 (SEM) | 53.67 +/− 6.41 (SEM) | 84.82 +/− 7.65 (SEM) | P = 0.0003 | P = 0.3237 |

Pre-Inflammatory Exposure (exposure 1 hour after inflammatory insult)

| | | | | | |
|---|---|---|---|---|---|
| 9 | 104.26 +/− 11.08 (SEM) | 21.10 +/− 6.82 (SEM) | 76.32 +/− 7.95 (SEM) | P < 0.0001 | P = 0.0566 |

NTI164 is more potent than CBD alone in suppressing COX-2 expression in human microglial cells.
DAPI cell viability stain: No cell death was detected and assessed as per the DAPI cell staining method.
Cells were viable throughout these in vitro studies.
Positive control/Inflammatory activation: Interleukin and Interferon gamma

### D.3.2 IL-2 and TNF-Alpha

NTI164 is statistically more potent in suppressing the key biomarkers: IL-12 and TNF-alpha when compared CBD alone and CBD|THC (1:1) mixture.

These results demonstrate that: NTI164 IL-12 P=0.0011 versus CBD alone was highly significant; NTI164 TNF-alpha P=0.0575 versus CBD alone was a positive trend; NTI164 IL-12 P=0.0069 versus CBD\THC combination Highly significant; NTI164 TNF-alpha P=0.0446 versus CBD\THC combination was significant.

TABLE 8
outlines the significance between NTI164 versus CBD alone and
CBD \ THC combinations (1:1 concentration ratio) in suppressing
TNF-alpha and IL-12.

| | | | Statistical |
|---|---|---|---|
| | | Results | Significance |
| | | Expressed as: | Reduction |
| | | Average +/− | Compared to |
| | | Standard | Control |
| Treatment | Cytokine | Deviation (SD) | Alone |
| Control: Interleukin | IL-12 | 99.91 +/− 12.88 | |
| and | TNF -alpha | | |
| Interferon Activation | IL-12 | 56.30 +/−18.24 | *P = 0.0001 |
| NTI164 (2μg/ml) | TNF -alpha | 58.28 +/− 15.08 | *P <0.0001 |
| CBD alone (2μg/ml) | IL-12 | 84.40 +/− 6.54 | P = 0.008 |
| | TNF -alpha | 71.13 +/− 12.81 | P = 0.0005 |
| CBD \| THC (1:1) | IL-12 | 80.77 +/− 12.23 | P = 0.008 |
| (2μg/ml) | TNF -alpha | 74.49 +/− 14.30 | P = 0.01 |

### D.4 Discussion

### D.4.1 COX-2, IL2 and TNF-Alpha

These results, showing suppressing COX-2, IL2 and TNF-alpha, reconfirm the potent properties of NTI164 in modulating inflammatory processes in neurological disorders where inflammation induced by immune responses is dysregulated.

### E Example 5-Further Characterisation of Nti164

### E.1 Study Aim

The compositional analysis of NTI164 extracted via oil, using UPLC/MS methods (as outlined in the above examples).

### E.2 Materials and Methods

The ACQUITY UPLC H-Class System combined with the CORTECS UPLC Shield RP18 particle chemistry was used to provide a UPLC isocratic separation of main cannabinoids in a 10.5-minute cycle time. Samples of NTI164 were assayed on a weekly basis and CBDA was used as a main marker as a stability indicator. Results presented in Table 6 demonstrate that NTI164 is stable at room temperature within an inert oil media over 6 weeks. There is no decarboxylation or product degradation observed over this time frame

Equipment: The following equipment was used: 10 mL glass scintillation bottles with lids; Cobram's Estate olive oil; plant grinder (similar to a coffee or food grade grinder) pore size up to 50 μM-80 μM; Whatman paper, grade 1; pipettes; weight scale (transfer boats and spoons); Eppendorf tubes; 50 mL falcon tubes; bench top centrifuge (Eppendorf Centrifuge 5702); Oz Design Brand 6 Litre Fruit, Wine and Cider Press.

Extraction: Pressing and Centrifugation: All work is undertaken at standard lab temperatures (18-22 °C). The buds of NTI164 were stripped off hard stalks and the stalks discarded. The grinder was cleaned with 70% EtOH and the grinding compartment was filled with dried plant material. The material was ground on the finest of the three setting for 10 seconds (1-2 mm particle size). The grounds were then mixed with 100 ml of olive oil in an autoclaved Schott bottle at a ratio plant/oil of 333 mg/ml. It was then placed on a stirrer at room temperature for 1 hour, stirred with magnetic flea (50 rpm). The oil plus plant mixture is then put into the Oz Design Brand 6 Litre Fruit, Wine and Cider Press to reclaim the oil component from the plant (the mash). The reclaimed oil was then placed into 50 mL falcon tubes and spun at 300 g for 15 minutes at room temperature (Isolation 1). The oil was then removed into a clean Schott bottle and keeping track of the volume reclaimed. The recovery of the oil for Isolation 1 is approximately 40%. The mash is discarded following each isolation. To the reclaimed oil, we added a further 333 mg/ml ground plant/oil (a further 100 ml) material and repeated the 1 hour mix, and reclaimed and re-used oil, until a total of 999 μg/mL (3×100 ml) of plant/oil mixture passed through (Isolation 2). The recovery of the oil for Isolation 2 is approximately 50%. For the final time, we placed into falcon tubes and spin as discussed above (Isolation 3). The recovery of the oil at for Isolation 3 is approximately 50%. We then collected the oil only and placed the oil into Eppendorf tubes for processing. This triplicate extraction method resulted in a total volume of 50 ml of final product at a concentration of 48 mg of CBDA to 1 ml of olive oil determined using UPLC potency testing using the methods described below.

### E.3 Results

The results of the UPLC analysis are presented below in Table 9.

TABLE 9
presents the NTI164 composition extracted using the ethanol
extraction and the components quantified using the UPLC
methods herein described

| | | Oil Extract | Oil |
|---|---|---|---|
| | | 18/11/21 | Extract |
| | | (NTI164) | 07/12/21 |
| CANNABINOIDS | Cas No's | % w/w | % w/W |
| Cannabidiol (CBD) | 13956-29-1 | 5.80 | 5.34 |
| Cannabidiol acid (CBD-A) | 1244-58-2 | 62.78 | 60.29 |
| Tetrahydrocannabinol (D9-THC) | 1972-08--03 | 0.70 | 0.65 |
| (Tetrahydrocannabinolic acid) | 23978-85-0 | 2.01 | 1.99 |
| (D9- THC-A) | | | |
| Cannabidivarin (CBDV) | 24274-48-4 | 0.49 | 0.48 |
| Cannabigerol Acid( CBG-A) | 25555-57-1 | 1.26 | 1.14 |
| Cannabigerol (CBG) | 25654-31-3 | 0.44 | 0.39 |
| Tetrahydrocannabivarin (THCV) | 28172-17-0 | 0.26 | 0.26 |
| Cannabinol (CBN) | 521-35-7 | 1.98 | 2.28 |
| DELTA-8-THC (D8-THC ) | 5957-75-5 | <0.1 | <0.1 |
| Cannabichrome (CBC) | 20675-51-8 | 0.81 | 0.85 |
| Total THC = D9-THC + | % amount | 2.45 | 2.40 |
| (D9-THC-A*0.877) | | | |
| Total CBD = CBD + | % amount | 60.85 | 58.22 |
| (CBD-A*0.877) | | | |

By expanding the pore size in the grinder system from 50 μM to 80 μM, we were able to extract cannabinol (CBN) 1-3% in the final oil extract product.

### F Example 6-Assessing the Anti-Inflammatory Properties of NTI164 in Combination with Diclofenac

### F.1 Study Aim

To assess the anti-inflammatory properties of NTI164 combined with diclofenac compared to CBD combined with diclofenac.

Diclofenac is a nonsteroidal anti-inflammatory drug (NSAID). Diclofenac is used to treat mild to moderate pain, or signs and symptoms of osteoarthritis, rheumatoid arthritis and neuropathic pain.

### F.2 Materials and Methods

### F.2.1 Immunohistochemistry (Protein Level) Assay

Human derived microglial cells were fixed for 10 min with 4% paraformaldehyde (PFA) in PBS. After 3×5 minutes washing with PBS, cells were incubated with primary antibodies (anti-COX2) 1:1000 overnight at 4 °C, and after 3×5 minutes washing in PBS, cells were then incubated in appropriate fluorescent secondary antibody 1:250 (Invitrogen) for 2 hours at room temperature. After a final wash, as previous, cells nuclei were sained with DAPI in the mounting media. Photomicrographs were taken of the cells in three fields of view per well from duplicate wells and analysed using Fiji for area coverage of each marker.

COX-2 is measured using the immunohistochemistry assay—as described above. As part of the positive control, cells are treated with Interleukins and Interferons which cause the cells to enter into an “active inflammatory state”. In the treatment groups, these cells are also treated with various treatments and COX-2 expression is then assessed following these treatments (1 hr after treatment).

### F.2.2 Sample Preparation and Dilutions

The following samples were prepared: CBD alone, NTI164 alone, Diclofenac alone, Diclofenac+NTI164 were prepared.

In this example, ethanol extraction was used. 500 mg of dried plant material NTI164 is suspended in 20 ml of absolute ethanol (using 50 ml blue top falcon tubes appropriate for centrifugation) and stirred/shaken vigorously for 60 seconds. The tubes are then placed in a sonication bath for 10 mins at 35-40 C. Upon completion of the sonication samples are then placed in a tray shaker (200 rpm) for 30 minutes at room temperature. Once completed the samples are then centrifuged at 4400 rpm for 5 minutes. The supernatant is collected for testing and development.

Units used to describe treatments for test products and concentrations for NTI164.
a. 1/1000 dilution of extract—10 UL (stock material is NTI164—10 UL, which equates to 2 μg/ml of CBDA)
b. 1/3000 dilution of extract—3 UL (stock material is NTI164—3 UL, which equates to 6 μg/ml of CBDA)
c. 1/10000 dilution of extract—1 UL (stock material is NTI164—1 UL, which equates to 0.1 μg/ml of CBDA).

For the CBD sample, a pure standard (in powder form) was used. CBD 98% isolate was purchased as a reference standard from LGC Standards (London UK) (CAS No. 13956-29-1). The CBD standard reference was prepared at concentration of 1 mg/ml (in acetonitrile). CBD dilutions were made in acetonitrile as follows: 2 μg/ml; 6 μg/ml; and 0.1 μg/ml.

Diclofenac (CAS number 15307-86-5) was purchased from Merck Chemicals (purity, 98% HPLC). Reference stock standard at 1 mg/ml was created using acetonitrile and the following dilutions were made: 2 μg/ml; 6 μg/ml; 0.1 μg/ml.

The final concentrations of NTI164 (CBDA equivalent), CBD and Diclofenac that was used in this study was 2 μg/ml.

### F.3 Results

### F.3.1 Synergistic Effects

Synergistic effects were observed with the NTI164+diclofenac combination. COX-2 inflammation was down to 8% with the NTI164+diclofenac combination versus 24% with diclofenac alone and 55% with NTI164 alone.

Treatment with NTI164 alone is very effective in reducing the cellular inflammation process but when combined with diclofenac these anti-inflammatory effects are even stronger.

TABLE 10
presents the inflammation results for the combination study
(N = 9), NTI164 plus diclofenac.

| Inflammation | | | DICLO | |
|---|---|---|---|---|
| activation (I&I)- | NTI164 | DICLO | (2 μg/ml) + | CBD |
| COX-2 | alone | Alone | NTI164 | alone |
| ACTIVITY | (2 μg/ml) | (2 μg/ml) | (2 μg/ml) | (2 μg/ml) |
| 156.06 | 62.88 | 13.12 | 4.81 | 73.8 |
| 99.58 | 74.36 | 12.78 | 1.22 | 42.4 |
| 57.35 | 92.57 | 18.17 | 11.23 | 44.92 |
| 23.07 | 29.29 | 28.761 | 9.871 | 109.99 |
| 147.5 | 53.04 | 32.01 | 6.98 | 136.99 |
| 70.58 | 73.86 | 24.92 | 4.23 | 117.84 |
| 64.92 | 43.99 | 56.19 | 18.65 | 43.14 |
| 125.6 | 25.64 | 11.9 | 9.192 | 77.81 |
| 167.89 | 47.4 | 22.19 | 8.76 | 59.02 |
| Mean 101.39 | 55.89 | 24.44 | 8.32 | 78.43 |

### G Example 7-Assessing the Anti-Inflammatory Properties of NTI164 in Combination with Celecoxib

### G.1 Study Aim

To assess the anti-inflammatory properties of NTI164 combined with diclofenac compared to CBD combined with celecoxib.

Celecoxib is a nonsteroidal anti-inflammatory drug (NSAID). Diclofenac is used to treat mild to moderate pain, or signs and symptoms of conditions such as osteoarthritis or rheumatoid arthritis.

### G.2 Materials and Methods

### G.2.1 Microglial BV2 Culture

The immortalized microglia cell line, BV2, was purchased from the American Tissue Culture Collection. BV2 were cultured in RPMI media containing gentamycin and supplemented with 10% fetal bovine serum (FBS) for expansion and 5% FBS when plated for experiments. All cells were from between passage numbers 39 and 45. Cells were plated at 45,000 cells/mm2 and treated 24 hours after plating with phosphate buffered saline (PBS, as a control) or interleurkin-1B+interferon-γ (IL-1B+IFNγ, to induce inflammation). To test the effects of NTI164 to alter the inflammatory response, NTI164 and synergy treatments (see below) were applied one hour after inflammation. NTI164 extracted were applied at 10 μL, 3 μL or 1 μL from isolated obtained using the original extraction protocol: range determined from mass spec data=1.0-0.1 ug CBDA. Treatments were celecoxib 5, 25, and 125 μM.

### G.2.2 Multiplex Cytokine/Chemokine Assay

Microglia media harvested following treatment initiation was centrifuged briefly to remove particulates (300 g for 10 min). Cytokine and chemokine levels in the microglial media were measured using a Bio-Plex 200 with a 96-well magnetic plate assay according to the manufacturer's instructions (Bio-Rad). Cytokines and chemokines measured included IL-1α, IL-1β, IL-2, IL-6, IL-10, IL-12 (p70), IL-13, G-CSF, GM-CSF, IFNγ, TNFα, CXCL1 (KC), CCL2 (MCP-1), and CCL5 (RANTES). All samples were run in duplicate that were averaged for analysis, at least three samples per group were run.

### G.2.3 Immunohistochemistry (Protein Level) Assay

Cells were fixed for 10 min with 4% paraformaldehyde (PFA) in PBS. After 3×5 minutes washing with PBS, cells were incubated with primary antibodies (anti-COX2) 1:1000 overnight at 4 °C, and after 3×5 minutes washing in PBS, cells were then incubated in appropriate fluorescent secondary antibody 1:250 (Invitrogen) for 2 hours at room temperature. After a final wash, as previous, cells nuclei were sained with DAPI in the mounting media. Photomicrographs were taken of the cells in three fields of view per well from duplicate wells and analysed using Fiji for area coverage of each marker.

### G.2.4 Cell Viability (Mitochondrial Activity) Assay

Microglial viability was quantified using MTT [3-(4,5-dimethylthiazol-2-yl-)-2,5-diphenyl-2H-tetrazolium bromide; Sigma]. In this assay MTT, a tetrazolium dye, is bioreduced by the mitochondria into a formazan product that is insoluble in tissue culture medium. In brief, MTT was added to a final concentration of 250 μg/ml to cells at various time points following treatment with PBS, LPS or IL-4 with or without test product. After 30 min, formazan was dissolved in DMSO and the absorbance was measured at 490 nm using a spectrophotometer (Glomax Multi+; Promega, UK).

### G.3 Results

### G.3.1 Synergistic Effects

Synergistic effects were observed with the NTI164+celecoxib combination. Inflammation observed by MTT assay, cytokine release (TNFalpha, G-CSF, IL1a, IL6) and COX2 protein, was down with the NTI164+celecoxib combination versus celecoxib alone and NTI164 alone.

Treatment with NTI164 alone is very effective in reducing the cellular inflammation process but when combined with celecoxib these anti-inflammatory effects are even stronger.

TABLE 11
presents the inflammation results for the combination study to
demonstrate synergistic effects of NTI164 and Celebrex.

| | | | | | | Significance | % Reduction in |
|---|---|---|---|---|---|---|---|
| | | Inflammation | | | Celebrex | Celebrex + | inflammation |
| | | only: | | | (5 uM) + | NTI164 | using |
| | Control | Interleukin & | Celebrex | NTI164 | NTI164 | versus | combination |
| Biomarker | PBS | Interferon | 5 uM | 7.5 uM | (7.5 | Celebrex | therapy versus |
| Analysis | buffer | activation | concentration | concentration | ug/ml) | Alone | Celebrex alone |
| COX-2 | 0.65 | 1 | 0.768 | 0.691 | 0.586 | P = 0.0210 | 28% |
| Protein | | | | | | Significant | |
| +/−SEM | 0.034 | 0 | 0.057 | 0.021 | 0.200 | | |
| TNF-a | 19.33 | 45 | 30.17 | 27.23 | 19.33 | P = 0.0105 | 30% |
| | | | | | | Significant | |
| +/−SEM | 7.01 | 8.35 | 1.53 | 2.23 | 2.02 | | |
| IL-6 | 10.50 | 366.33 | 128.50 | 92.12 | 31.13 | P = 0.0002 | 77% |
| | | | | | | Highly | |
| | | | | | | Significant | |
| +/−SEM | 2.77 | 60.41 | 10.53 | 11.12 | 25.27 | | |
| IL-1a | 67.5 | 154.83 | 112.5 | 87.23 | 59.00 | P = 0.0213 | 43% |
| | | | | | | Significant | |
| +/−SEM | 34.66 | 33.11 | 27.54 | 13.44 | 22.52 | | |
| GM-CSF | 188.80 | 768.13 | 511.97 | 323.45 | 268.17 | P = 0.0398 | 47% |
| | | | | | | Significant | |
| +/−SEM | 73.19 | 214.36 | 275.14 | 112.12 | 138.58 | | |

### H Example 8-Assessing the Anti-Inflammatory Properties of NTI164 in Combination with Prednisone

### H.1 Study Aim

To assess the synergistic suppression of the inflammatory response with combined prednisone and NTI164 treatment in preclinical in-vitro studies.

### H.2 Materials and Methods

Cells were fixed for 10 min with 4% paraformaldehyde (PFA) in PBS. After 3×5 minutes washing with PBS, cells were incubated with primary antibodies. (anti-COX2) 1:1000 overnight at 4 °C, and after 3×5 minutes washing in PBS, cells were then incubated in appropriate fluorescent secondary antibody 1:250 (Invitrogen) for 2 hours at room temperature. After a final wash, as previous, cells nuclei were stained with DAPI in the mounting media. Photomicrographs were taken of the cells in three fields of view per well from duplicate wells and analysed using Fiji for area coverage of each marker.

Treatment groups include:
(1) Control: PBS Buffer
(2) Positive control: Inflammatory stimulation by Interferon gamma and Interleukin-1B activation Prednisone (PDN) concentration 5 uM
(3) Combination therapy: Prednisone (PDN) 5 uM+NTI164 concentration 7.5 ug/ml

### H.3 Results

Result Analysis: Calculated as % reduction in inflammation, PDN versus Combination therapy (prednisone 5 uM+NTI164 concentration 7.5 ug/ml). Student's t-test was used for statistical analysis.

Treatment with NTI164 alone is very effective in reducing the cellular inflammation process but when combined with prednisone these anti-inflammatory effects are even stronger.

TABLE 12
presents the inflammation results for the combination study to demonstrate
synergistic effects of NTI164 and Prednisone

| | | Inflammation | | | | | % Reduction in |
|---|---|---|---|---|---|---|---|
| | | only: | | | PDN | Significance | inflammation |
| | Control | Interleukin & | | NTI164 | (5 uM)+ | PDN vs | using combination |
| Biomarker | PBS | Interferon | PDN 5 uM | 7.5 ug/ml | NTI164 | PDN + | therapy versus |
| Analysis | buffer | activation | concentration | Concentration | (7.5 ug/ml) | NTI164 | PDN alone |
| COX-2 | 0.799 | 1 | 0.888 | 0.712 | 0.586 | P = 0.0210 | 34% |
| Protein | | | | | | Significant | |
| +/−SEM | 0.075 | 0 | 0.057 | 0.091 | 0.200 | | |
| TNF-a | 20.33 | 45 | 30.17 | 24.56 | 20.33 | P = 0.0105 | 33% |
| | | | | | | Significant | |
| +/−SEM | 5.01 | 8.35 | 1.53 | 1.45 | 2.02 | | |
| IL-6 | 9.50 | 366.33 | 228.50 | 98.98 | 30.33 | P = 0.0002 | 87% |
| | | | | | | Highly | |
| | | | | | | Significant | |
| +/−SEM | 2.77 | 60.41 | 11.53 | 9.82 | 15.27 | | |
| IL-1a | 77.5 | 154.83 | 144.5 | 82.23 | 69.00 | P = 0.0213 | 53% |
| | | | | | | Significant | |
| +/−SEM | 38.66 | 33.11 | 27.54 | 18.92 | 22.52 | | |
| GM-CSF | 168.80 | 768.13 | 611.97 | 315.12 | 278.97 | P = 0.0398 | 54% |
| | | | | | | Significant | |
| +/−SEM | 83.29 | 294.36 | 375.14 | 112.12 | 138.58 | | |

### I Example 9-Assessing the Anti-Inflammatory Properties of NTI164 in Combination with Psylocibin

### I.1 Study Aim

To assess the synergistic suppression of the inflammatory response with combined psylocibin and NTI164 treatment in preclinical in-vitro studies.

### 1.2 Materials and Methods

Microglia media harvested following treatment initiation was centrifuged briefly to remove particulates (300 g for 10 min). The level of TNFα in the microglial culture media was measured using a Bio-Plex 200 with a 96-well magnetic plate assay according to the manufacturer's instructions (Bio-Rad). The samples were run in duplicate that were averaged for analysis. At least three samples per group were run.

### I.3 Results

### Result Analysis:

TABLE 13
TNF-Alpha assessments
Table 13 presents the TNF-Alpha assessments
for the psylocibin combination study

| | TNF-ALPHA |
|---|---|
| | ASSESSMENTS |
| SAMPLE | RESULTS |
| PBS Control | 1.00 |
| Positive activation I&I | 10.67 |
| NTI164 5 ug/ml | 2.07 |
| NTI164 1.75 ug/ml | 15.87 |
| SubP (Psylocibin) 5 ug/ml | 9.17 |
| SubP (Psylocibin) 1 ug/ml | 4.87 |
| NTI164 + SubP 5 ug/ml | 1.67 |
| NTI164 + SubP 1 ug/ml | 1.73 |

Treatment with NTI164 alone is very effective in reducing the cellular inflammation process but when combined with psylocibin these anti-inflammatory effects are even stronger.

### J Example 10-NTI164 for Treatment of ASD Level II/III-4 Weeks

### J.1 ASD Study Design and Methods

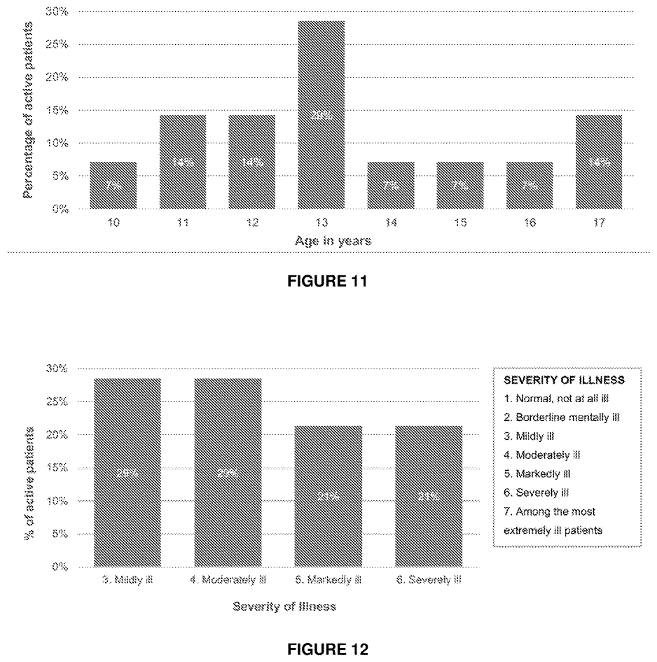

Of the 18 patients who were enrolled into the study, patients who received NTI164 made up 94% (n=17). Active patients made up 78% (n=14), patients who discontinued after receiving their first dose of NTI164 made up 16% (n=3) and patients who discontinued prior to receiving NTI164 but after enrolment made up 6% (n=1). The mean age of active patients was 13.4 years of age with the youngest patient being 10 years and the oldest being 17 years of age (FIG. 11).

All active patients were diagnosed with ASD Level II/III and were assessed at baseline as being either ‘Mildly ill’, ‘Moderately ill’, ‘Markedly ill’ or ‘Severely ill’ on the CGI Severity scale (FIG. 12).

Patients commenced treatment of NTI164 at 5 mg/kg/day and was increased weekly by 5 mg/kg/day for a period of 4 weeks until 20 mg/kg/day or the maximum tolerated dose was achieved The daily dose was calculated by multiplying the dosage by the patient's weight and then dividing by the concentration of CBDA in the oil (53 mg/mL). This returned a total daily volume in mL (Table 14) which was split into twice daily (BD) AM and PM doses.

TABLE 14
Calculation of daily dose for each patient.

| Patient | | Daily Dose in mL | | | |
|---|---|---|---|---|---|
| ID | Weight | 5 mg/kg | 10 mg/kg | 15 mg/kg | 20 mg/kg |
| 102 | 56.9 | 5.4 | 10.7 | 16.1 | 21.4 |
| 103 | 57.8 | 5.4 | 10.8 | 16.3 | 21.7 |
| 104 | 57.4 | 5.3 | 10.6 | 15.8 | 21.7 |
| 106 | 41.0 | 3.8 | 7.7 | 11.5 | 15.4 |
| 110 | 91.7 | 8.7 | 17.3 | 26.0 | 34.0 |
| 111 | 41.1 | 3.9 | 7.9 | 11.8 | 15.7 |
| 112 | 101.1 | 9.5 | 19.1 | 28.6 | 38.2 |
| 113 | 44.7 | 4.2 | 8.4 | 12.7 | 16.0 |
| 114 | 89.8 | 8.5 | 16.9 | 25.4 | 33.9 |
| 115 | 40.5 | 3.7 | 7.5 | 11.2 | 14.9 |
| 116 | 54.5 | 5.1 | 10.2 | 15.2 | 20.3 |
| 117 | 65.5 | 6.3 | 12.6 | 18.8 | 24.7 |
| 118 | 52.4 | 4.9 | 9.9 | 14.8 | 19.8 |
| 119 | 68.9 | 6.5 | 13.0 | 19.5 | 26.0 |

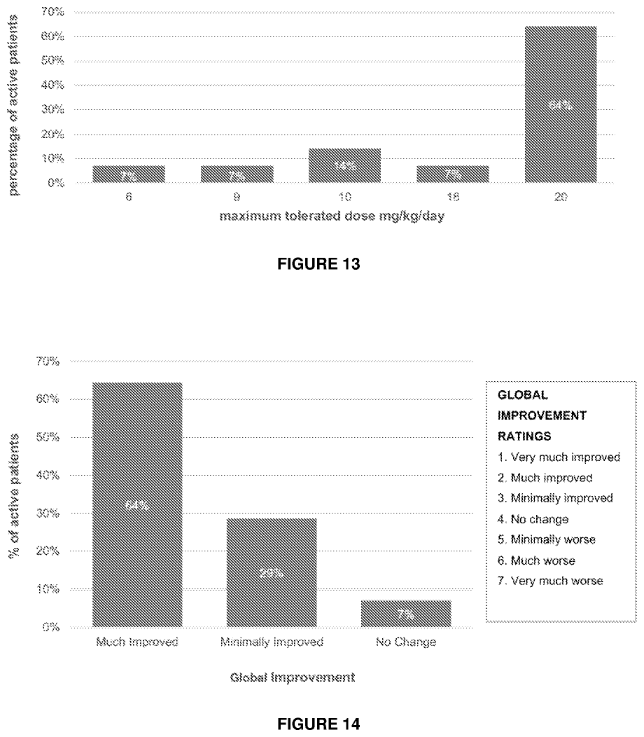

The average maximum daily dose for active patients was 16.7 mg/kg/day with 64% of patients tolerating the maximum dose of 20 mg/kg/day and 36% of patients tolerating a maximum daily dose ranging between 6 mg/kg/day to 19 mg/kg/day (FIG. 13).

### J.2 Study Results

The Clinical Global Impression-Severity (CGI-S) scale was used to assess:
Global Improvement: rates the total improvement whether or not, in the clinician's judgement, is due entirely to drug treatment;
Severity of Illness: a comparison of baseline and post-baseline (28-days NTI164 treatment); and
Efficacy Index: rated based on drug effect only. This is a calculated score based on the degrees of therapeutic effect and side effects.

### Global Improvement

93% of active patients showed improvement after 28 days of daily treatment with NTI164. 64% of these patients had a global improvement of ‘Much improved’, 29% had a global improvement of ‘Minimally improved’ and only one patient (7%) had ‘No change’ (FIG. 14). The Wilcoxon Signed-Rank Test and the Paired t-test were used to assess the statistical significance:

Paired t-test: the mean difference of CGI-S between 28 days of treatment and baseline was-0.714, 95% confidence interval=−1.332, −0.097, p value=0.027. The Wilcoxon Signed-Rank Test statistic was: −15, the corresponding p-value was 0.047.

### Severity of Illness

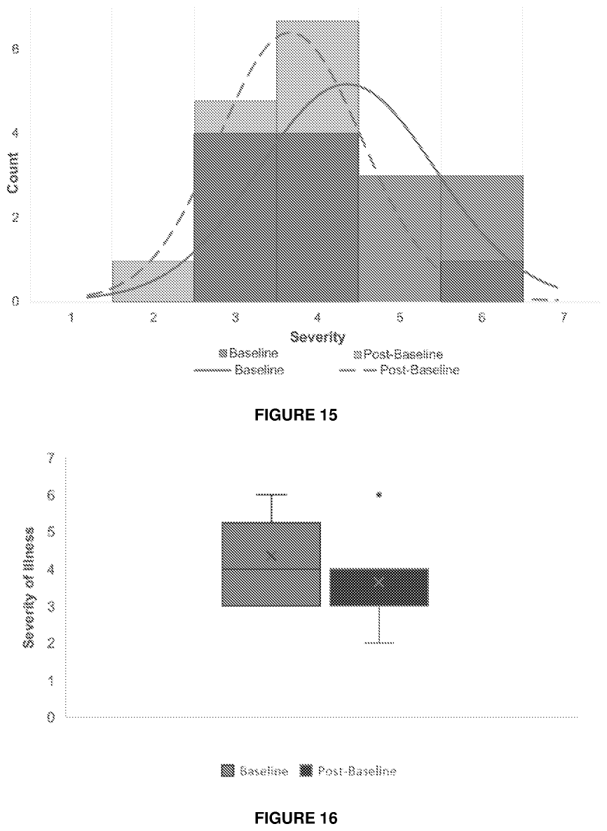

The average rating for the severity of illness at baseline was 4.4 (FIG. 12). This reduced to an average rating of 3.6 after 28 days of NTI164 treatment (FIG. 15 and FIG. 16).

### Therapeutic Effect

After 28-days of daily treatment with NTI164, 14% of active patients demonstrated the second highest possible efficacy index of 2: Marked therapeutic effect with side effects that do not significantly interfere with patient's functioning.

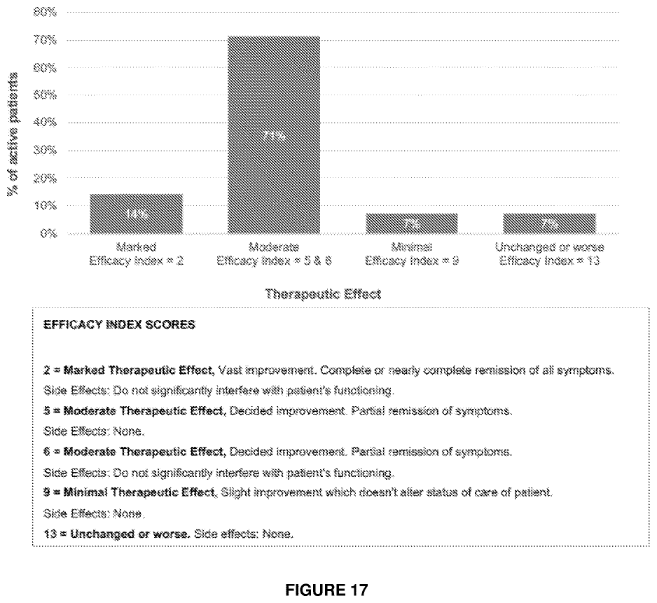

72% of active patients had an efficacy index of either 5 or 6: Moderate therapeutic effect with half of these patients having no side effects and the other half having side effects that do not significantly interfere with patient's functioning, 7% had an efficacy index of 9: Minimal therapeutic effect with no side effects and only one patient, 7%, had an efficacy index based on seeing no change in condition, 13: Unchanged or worse with no side effects (FIG. 17).

### K Example 11-NTI164 for Treatment of ASD Level II/III-20 Weeks

### K.1 ASD Study Design and Methods

Example 10 above presents the results of the treatment at the 4 weeks (28 days) time point (n=14 active). This Example 11 presents the results of the treatment at the 20 week time point (n=12 active). As discussed in Example 10 above, patients commenced treatment of NTI164 at 5 mg/kg/day which was increased weekly by 5 mg/kg/day for a period of 4 weeks until 20 mg/kg/day or the maximum tolerated dose was achieved and (in this study) continued their maximum tolerated dose for 16 weeks (providing a total daily dosing period of 20 weeks).

The overall purpose of this study was to assess the continued safety and efficacy of NTI164 administered daily over a 20-week period. The secondary objective was to assess the efficacy of NTI164 in the treatment of symptoms associated with autism spectrum disorder. Efficacy was measured with various physician-led and parent-led standard questionnaires used in the art.

Patients (n=12)

The mean age of active patients at week 20 was 13.3 years of age with the youngest patient being 10 years and the oldest being 17 years of age (Error! Reference source not found). All active patients were diagnosed with ASD Level II/III and were assessed at baseline as being either ‘Mildly ill’, ‘Moderately ill’, ‘Markedly ill’ or ‘Severely ill’ on the CGI Severity scale (Error! Reference source not found.1).

### Dose

Based on paediatric trials undertaken around the world, the selected maximum dose for this study was 20 mg/kg/day.

To reduce the risk of side-effects, the study drug was up-titrated over the course of four weeks commencing at 5 mg/kg/day and increasing weekly by 5 mg until the maximum tolerated dose or 20 mg/kg/day was achieved. The maximum tolerated dose was then administered over the course of 20 weeks. The daily volume was administered over two doses, AM and PM.

The formula used to calculate each patient's dose was: Weight×Dose/NTI164 Concentration=Daily Dose/2=Twice-Daily Dose. During the first week of treatment, each patient received 5 mg/kg/day of NTI164. During the second week of treatment, each patient received 10 mg/kg/day of NTI164. During the third week of treatment, each patient received 15 mg/kg/day of NTI164. During the fourth week of treatment, each patient received 20 mg/kg/day of NTI164. During weeks 5-20 of treatment, each patient received their maximum tolerated dose or 20 mg/kg/day of NTI164.

NTI164 was prepared in oil for oral administration. The total concentration of the oil was 53 mg/ml.

At the end of week 20, participants had the option of either ending their participation and down-titrating 5 mg/kg/week until they ceased the study drug or continuing their maximum tolerated dose up to week 52.

### Primary Endpoints

Safety was monitored and measured using standard steps in the art. Safety was monitored and measured using full blood examinations, liver and kidney function tests and vital signs in addition to parent/carer and physician questionnaires completed at baseline and every four weeks up until week 20.

### Secondary Endpoints

Efficacy was monitored and measured using standard steps in the art.

Clinical Global Impression Scale-Severity of Illness (CGI-S). Reflects clinician's impression of severity of illness on a 7-point scale ranging from 1=not at all to 7=among the most extremely ill. [Time Frame: Baseline, Week 4, Week 8, Week 12, Week 20].

Vineland Adaptive Behaviour Scales, Third Edition (Vineland-3). Used to measure adaptive functioning across three core domains (Communication, Daily Living Skills, and Socialization), and two optional domains (Motor Skills and Maladaptive Behaviour); items are rated on a 3-point scale (0=never; 1=sometimes; 2=usually or often). The core domains sum to a total Adaptive Behaviour Composite. [Time Frame: Baseline, Week 20].

Social Responsiveness Scale, 2nd Edition-School-Age Form (SRS-2). Five domains are assessed including: Social Awareness, Social Cognition, Social Communication, Social Motivation, and Restricted Interests and Repetitive Behaviour. Items are scored on a 4-point scale (ranging from 1=not true to 4=almost always true). [Time Frame: Baseline, Week 20].

Clinical Global Impression Scale-Improvement-Caregiver (CGI-I-Ca). This is a 7-point scale measuring symptom change from baseline. Provided as baseline and post-baseline Caregiver and Clinician questionnaires. [Time Frame: Baseline, Week 4, Week 8, Week 12, Week 20].

Clinical Global Impression Scale-Improvement-Clinician (CGI-I-CI). This is a 7-point scale measuring symptom change from baseline. Provided as baseline and post-baseline Caregiver and Clinician questionnaires. [Time Frame: Baseline, Week 4, Week 8, Week 12, Week 20].

Clinical Global Impression Scale-Change in Target Behaviour (CGI-C). Reflects clinician's impression of change of behaviour on a 7-point scale ranging from 1=not at all to 7=very severe problem. Provided as Baseline and Post-Baseline questionnaires. [Time Frame: Baseline, Week 4, Week 8, Week 12, Week 20].

Clinical Global Impression Scale-Change in Attention (CGI-CA). Reflects clinician's impression of change in attention on a 7-point scale ranging from 1=not at all to 7=very severe problem. Provided as Baseline and Post-Baseline questionnaires. [Time Frame: Baseline, Week 4, Week 8, Week 12, Week 20].

Anxiety Scale for Children-Autism Spectrum Disorder-Parent Version (ASC-ASD-P). Parent/Caregiver form developed to detect symptoms of anxiety in youth with ASD. Composed of four subscales (Performance Anxiety, Uncertainty, Anxious Arousal, and Separation Anxiety), items are rated on a 4-point scale (0=never and 3=always). Subscales sum to equal a total score. [Time Frame: Baseline, Week 4, Week 8, Week 12, Week 20].

Anxiety Scale for Children-Autism Spectrum Disorder-Child Version (ASC-ASD-C). Child form developed to detect symptoms of anxiety in youth with ASD. Composed of four subscales (Performance Anxiety, Uncertainty, Anxious Arousal, and Separation Anxiety), items are rated on a 4-point scale (0=never and 3=always). Subscales sum to equal a total score. [Time Frame: Baseline, Week 4, Week 8, Week 12, Week 20].

Sleep Disturbance Scale for Children (SDSC). Six subscales including Disorders of Initiating and Maintaining Sleep, Sleep Breathing Disorders, Disorders of Arousal, Sleep Wake Transition Disorders, Disorders of Excessive Somnolence, and Sleep Hyperhydrosis. Items are rated on 5-point scale where 1=never and 5=always (daily). Subscale scores sum to equal a total score [Time Frame: Baseline, Week 4, Week 8, Week 12, Week 20].

### K.2 Study Results

### Safety Results

The safety data concludes that NTI164 at 5, 10, 15 and 20 mg/kg administered in two doses daily, is safe and well-tolerated in this study population. This conclusion is further supported by laboratory values. No changes were observed to patient's full blood examination, liver function or kidney function tests. Nor were there any changes observed to patient's vital signs.

### Efficacy Results

The Wilcoxon Signed-Rank Test and the Paired t-test were used to assess statistical significance of the analysed data sets.

Paired t-test: the mean difference of CGI-S between 20 weeks of treatment and baseline was-1.08, 95% confidence interval=−1.772, −0.3948, p value=0.005303.

The Wilcoxon Signed-Rank Test statistic was: −15, the corresponding p-value was 0.009654.

100% of patients (n=12) showed ‘much improved’ improvement of symptoms relating to severity of illness after 20 weeks of daily treatment with NTI164.

Table 15 below is a summary of results from the Wilcoxon Signed-Rank Tests and paired T-Tests on analysed datasets at week 20.

TABLE 15
Summary of Wilcoxon Signed-Rank Test and Paired T-Test on Analysed Datasets at 20 weeks

| | | Wilcoxon | |
|---|---|---|---|
| | | Signed- | Paired |
| Sub-DomainI | Scale | Rank Test | T-Test |
| Severity of illness | CGI-S | 0.010 | 0.005 |
| Adaptive behaviour composite (Total) | Vineland-3 | 0.003 | 0.001 |
| Communication | Vineland-3 | 0.004 | 0.002 |
| Daily living skills | Vineland-3 | 0.025 | 0.019 |
| Socialisation | Vineland-3 | 0.012 | 0.014 |
| Social responsive scale-Total T-score | SRS-2 | 0.013 | 0.012 |
| Social awareness-T-score | SRS-2 | 0.439 | 0.596 |
| Social cognition-T-score | SRS-2 | 0.036 | 0.028 |
| Social communication-T-score | SRS-2 | 0.018 | 0.019 |
| Social motivation-T-score | SRS-2 | 0.138 | 0.118 |
| Restricted interest and repetitive behaviour-T-score | SRS-2 | 0.014 | 0.009 |
| Social communication and interaction-T-score | SRS-2 | 0.021 | 0.029 |
| Anxiety scale for children-Child's total | ASC-ASD-C | 0.012 | 0.025 |
| Performance anxiety | ASC-ASD-C | 0.474 | 0.364 |
| Anxious arousal | ASC-ASD-C | 0.089 | 0.120 |
| Separation anxiety | ASC-ASD-C | 0.035 | 0.025 |
| Uncertainty | ASC-ASD-C | 0.035 | 0.033 |
| Anxiety scale for children-Parent's total | ASC-ASD-P | 0.053 | 0.034 |
| Performance anxiety | ASC-ASD-P | 0.096 | 0.070 |
| Anxious arousal | ASC-ASD-P | 0.229 | 0.333 |
| Separation anxiety | ASC-ASD-P | 0.033 | 0.025 |
| Uncertainty | ASC-ASD-P | 0.084 | 0.066 |
| Sleep disturbances scale for children-Total | SDSC | 0.018 | 0.016 |
| Disorders of initiating and maintaining sleep | SDSC | 0.026 | 0.010 |
| Sleep breathing disorders | SDSC | 0.042 | 0.047 |
| Disorders of arousal | SDSC | 0.480 | 0.522 |
| Sleep-wake transition disorders | SDSC | 0.072 | 0.094 |
| Disorders of excessive somnolence | SDSC | 0.706 | 1.000 |
| Sleep hyperhydrosis | SDSC | 1.000 | 1.000 |
| Anxiety, depression and mood scale-Total | ADAMS | 0.009 | 0.001 |
| Autism family experience questionnaire-Total | AFEQ | 0.888 | 0.961 |
| Experience of being a parent of a child with autism | AFEQ | 0.443 | 0.464 |
| Family life | AFEQ | 0.234 | 0.247 |
| Child development, understanding and social relationships | AFEQ | 0.765 | 0.799 |
| Child symptoms (feelings and behaviour) | AFEQ | 0.119 | 0.120 |

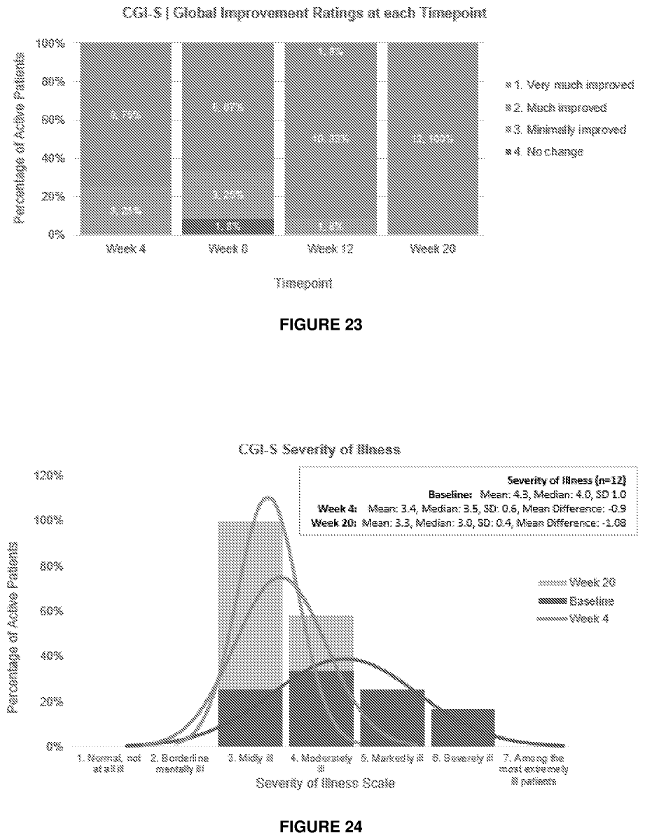

Global Improvement. 100% of active patients (n=12) showed improvement after 20 weeks of daily treatment with NTI164. All patients had a global improvement of ‘2. Much improved’. Three of these patients had previously scored ‘3. Minimally improved’ after 4 weeks of treatment. See FIGS. 22 and 23.

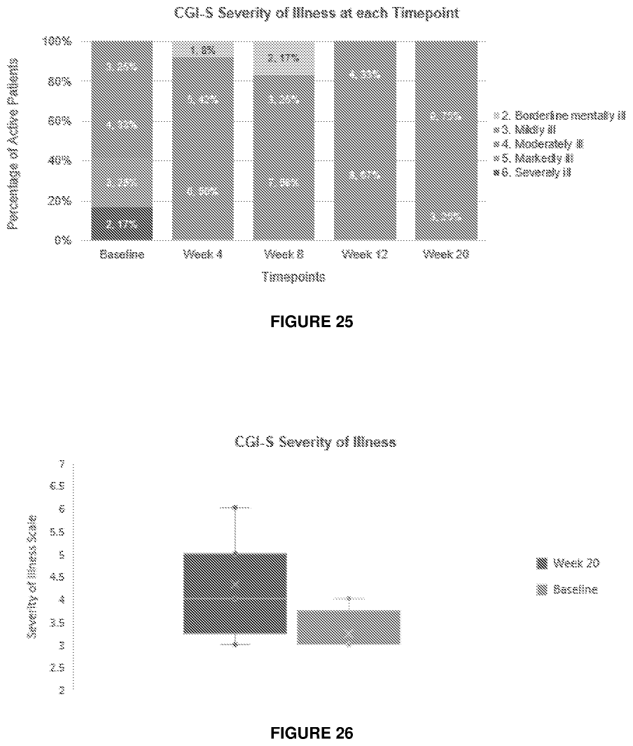

Severity of Illness. The average rating for the severity of illness at baseline was 4.3. This reduced to an average rating of 3.3 after 20 weeks of daily NTI164 treatment. See FIGS. 24 to 26.

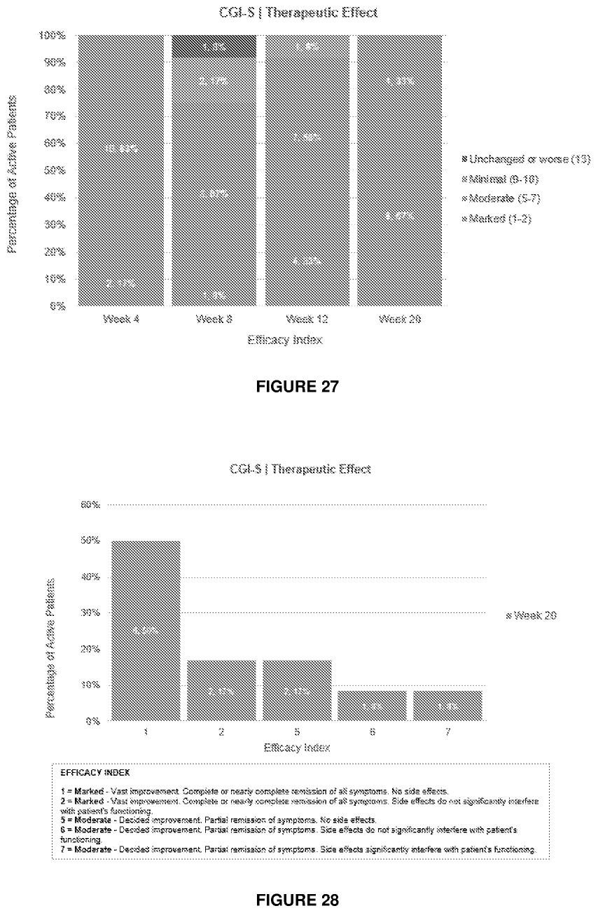

Therapeutic Effect. After 20 weeks of daily NTI164 treatment, 67% of active patients demonstrated the highest possible efficacy indexes of 1 and 2: Marked therapeutic effect-Vast improvement. Complete or nearly complete remission of all symptoms. 33% of patients had an efficacy index of either 5, 6 or 7: Moderate therapeutic effect-Decided improvement. Partial remission of symptoms. See FIGS. 27 to 28.

### Conclusion.

NTI164 was shown to be safe and well tolerated up to doses of 20/mg/kg/day. NTI164 has shown statistically significant efficacy in improving the symptoms associated with autism spectrum disorder after 20 weeks of daily therapy.

### Example 12-NTI164 for Treatment of ASD Level II/III-52 Weeks

### K.3 ASD Study Design and Methods

Example 10 above presents the results of the treatment at the 4 weeks (28 days) time point (n=14 active). Example above 11 presents the results of the treatment at the 20 week time point (n=12 active). This example presents the results of the treatment at the 52 week time point (n=11 active)

As discussed in Examples 10 and 11 above, patients commenced treatment of NTI164 at 5 mg/kg/day which was increased weekly by 5 mg/kg/day for a period of 4 weeks until 20 mg/kg/day or the maximum tolerated dose was achieved and (in this study) continued their maximum tolerated dose for 48 weeks (providing a total daily dosing period of 52 weeks).

The overall purpose of this study was to assess the continued safety and efficacy of NTI164 administered daily over a 52-week period. The secondary objective was to assess the efficacy of NTI164 in the treatment of symptoms associated with autism spectrum disorder. Efficacy was measured with various physician-led and parent-led standard questionnaires used in the art.

Patients (n=11)

The mean age of active patients at week 52 was 12.5 years of age with the youngest patient being 10 years and the oldest being 17 years of age. All active patients were diagnosed with ASD Level II/III and were assessed at baseline as being either ‘Mildly ill’, ‘Moderately ill’, ‘Markedly ill’ or ‘Severely ill’ on the CGI Severity scale.

### Dose

Based on paediatric trials undertaken around the world, the selected maximum dose for this study was 20 mg/kg/day.

To reduce the risk of side-effects, the study drug was up-titrated over the course of four weeks commencing at 5 mg/kg/day and increasing weekly by 5 mg until the maximum tolerated dose or 20 mg/kg/day was achieved. The maximum tolerated dose was then administered over the course of 48 weeks. The daily volume was administered over two doses, AM and PM.

The formula used to calculate each patient's dose was: Weight×Dose/NTI164 Concentration=Daily Dose/2=Twice-Daily Dose. During the first week of treatment, each patient received 5 mg/kg/day of NTI164. During the second week of treatment, each patient received 10 mg/kg/day of NTI164. During the third week of treatment, each patient received 15 mg/kg/day of NTI164. During the fourth week of treatment, each patient received 20 mg/kg/day of NTI164. During weeks 5-52 of treatment, each patient received their maximum tolerated dose or 20 mg/kg/day of NTI164.

NTI164 was prepared in olive oil (extra virgin) for oral administration. The total concentration of the oil was 53 mg/ml.

At the end of week 20, participants had the option of either ending their participation and down-titrating 5 mg/kg/week until they ceased the study drug or continuing their maximum tolerated dose up to week 52.

### Primary Endpoints

Safety was monitored and measured using standard steps in the art. Safety was monitored and measured using full blood examinations, liver and kidney function tests and vital signs in addition to parent/carer and physician questionnaires completed at baseline and every four weeks up until week 52.

### Secondary Endpoints

Efficacy was monitored and measured using standard steps in the art.

Clinical Global Impression Scale-Severity of Illness (CGI-S). Reflects clinician's impression of severity of illness on a 7-point scale ranging from 1=not at all to 7=among the most extremely ill. [Time Frame: Baseline, Week 4, Week 8, Week 12, Week 20, Week 52].

Vineland Adaptive Behaviour Scales, Third Edition (Vineland-3). Used to measure adaptive functioning across three core domains (Communication, Daily Living Skills, and Socialization), and two optional domains (Motor Skills and Maladaptive Behaviour); items are rated on a 3-point scale (0=never; 1=sometimes; 2=usually or often). The core domains sum to a total Adaptive Behaviour Composite. [Time Frame: Baseline, Week 20, Week 52].

Social Responsiveness Scale, 2nd Edition—School-Age Form (SRS-2). Five domains are assessed including: Social Awareness, Social Cognition, Social Communication, Social Motivation, and Restricted Interests and Repetitive Behaviour. Items are scored on a 4-point scale (ranging from 1=not true to 4=almost always true). [Time Frame: Baseline, Week 20, Week 52].

Clinical Global Impression Scale-Improvement-Caregiver (CGI-I-Ca). This is a 7-point scale measuring symptom change from baseline. Provided as baseline and post-baseline Caregiver and Clinician questionnaires. [Time Frame: Baseline, Week 4, Week 8, Week 12, Week 20, Week 52].

Clinical Global Impression Scale-Improvement-Clinician (CGI-I-CI). This is a 7-point scale measuring symptom change from baseline. Provided as baseline and post-baseline Caregiver and Clinician questionnaires. [Time Frame: Baseline, Week 4, Week 8, Week 12, Week 20, Week 52].

Clinical Global Impression Scale-Change in Target Behaviour (CGI-C). Reflects clinician's impression of change of behaviour on a 7-point scale ranging from 1=not at all to 7=very severe problem. Provided as Baseline and Post-Baseline questionnaires. [Time Frame: Baseline, Week 4, Week 8, Week 12, Week 20, Week 52].

Clinical Global Impression Scale-Change in Attention (CGI-CA). Reflects clinician's impression of change in attention on a 7-point scale ranging from 1=not at all to 7=very severe problem. Provided as Baseline and Post-Baseline questionnaires. [Time Frame: Baseline, Week 4, Week 8, Week 12, Week 20, Week 52].

Anxiety Scale for Children-Autism Spectrum Disorder-Parent Version (ASC-ASD-P). Parent/Caregiver form developed to detect symptoms of anxiety in youth with ASD. Composed of four subscales (Performance Anxiety, Uncertainty, Anxious Arousal, and Separation Anxiety), items are rated on a 4-point scale (0=never and 3=always). Subscales sum to equal a total score. [Time Frame: Baseline, Week 4, Week 8, Week 12, Week 20, Week 52].

Anxiety Scale for Children-Autism Spectrum Disorder-Child Version (ASC-ASD-C). Child form developed to detect symptoms of anxiety in youth with ASD. Composed of four subscales (Performance Anxiety, Uncertainty, Anxious Arousal, and Separation Anxiety), items are rated on a 4-point scale (0=never and 3=always). Subscales sum to equal a total score. [Time Frame: Baseline, Week 4, Week 8, Week 12, Week 20, Week 52].

Sleep Disturbance Scale for Children (SDSC). Six subscales including Disorders of Initiating and Maintaining Sleep, Sleep Breathing Disorders, Disorders of Arousal, Sleep Wake Transition Disorders, Disorders of Excessive Somnolence, and Sleep Hyperhydrosis. Items are rated on 5-point scale where 1=never and 5=always (daily). Subscale scores sum to equal a total score [Time Frame: Baseline, Week 4, Week 8, Week 12, Week 20, Week 52]

### K.4 Study Results

### Safety Results

The safety data concludes that NTI164 at 5, 10, 15 and 20 mg/kg administered in two doses daily, is safe and well-tolerated in this study population. This conclusion is further supported by laboratory values. No changes were observed to patient's full blood examination, liver function or kidney function tests. Nor were there any changes observed to patient's vital signs.

### Efficacy Results

The Wilcoxon Signed-Rank Test and the Paired t-test were used to assess statistical significance of the analysed data sets.

Paired t-test: the mean difference of CGI-S between 52 weeks of treatment and baseline was-1.1, 95% confidence interval=−2.080, −0.120, p value=0.0317.

Table 16 below is a summary of results from the paired T-Tests on analysed datasets at week 52.

TABLE 16
Summary of Paired T-Test on Analysed Datasets at 52 weeks

| | | Paired t-test | | | |
|---|---|---|---|---|---|
| | | Confidence | | Mean | Paired |
| Sub-Domain | Scale | Interval = 95% | | Difference | T-Test |
| Severity of illness | CGI-S | −2.080 | −0.120 | −1.1 | 0.03179 |
| Adaptive behaviour | Vineland-3 | 0.940 | 11.810 | 6.375 | 0.02756 |
| composite (Total) | | | | | |
| Communication | Vineland-3 | 4.264 | 8.236 | 6.25 | 0.0001442 |
| Daily living skills | Vineland-3 | 3.504 | 13.496 | 8.5 | 0.005041 |
| Socialisation | Vineland-3 | −2.131 | 15.131 | 6.5 | 0.1181 |
| Social responsive scale- | SRS-2 | −8.170 | −0.033 | −4.1 | 0.04852 |
| Total T-score | | | | | |
| Social awareness-T-score | SRS-2 | −4.213 | 9.213 | 2.5 | 0.4213 |
| Social cognition-T-score | SRS-2 | −7.216 | 0.816 | −3.2 | 0.1049 |
| Social communication-T-score | SRS-2 | −6.478 | 1.678 | −2.4 | 0.2158 |
| Social motivation-T-score | SRS-2 | −9.413 | −2.187 | −5.8 | 0.005474 |
| Restricted interest and | SRS-2 | −15.218 | 1.818 | −6.7 | 0.1089 |
| repetitive behaviour-T-score | | | | | |
| Social communication and | SRS-2 | −6.456 | 0.456 | −3.0 | 0.08113 |
| interaction-T-score | | | | | |

100% of patients (n=11) showed ‘much improved’ improvement of symptoms relating to severity of illness after 52 weeks of daily treatment with NTI164.

Vineland-3. The mean difference of the Adaptive Behaviour Composite domain of the Vineland-3 between 52 weeks of treatment and baseline was 6.375, 95% confidence interval=0.940, 11.810, p value=0.02756. See Table 17 for further presentation of the results.

TABLE 17
Vineland-3-The mean difference of the Adaptive Behaviour
Composite domain of the Vineland-3

| | P-value | P-value |
|---|---|---|
| | (Paired T-Test) | (Paired T-Test) |
| Vineland-3 Domain | 20 weeks | 52 weeks |
| Adaptive behaviour | 0.0005 | 0.0278 |
| composite | | |
| Communication | 0.002 | 0.0001 |
| Daily living skills | 0.019 | 0.0050 |
| Socialisation | 0.014 | 0.118 |

The mean difference of the Communication sub-domain of the Vineland-3 between 52 weeks of treatment and baseline was 6.25, 95% confidence interval=4.264, 8.236, p value=0.0001442.

The mean difference of the Daily Living Skills sub-domain of the Vineland-3 between 52 weeks of treatment and baseline was 8.5, 95% confidence interval=3.504, 13.496, p value=0.005041.

The mean difference of the Socialisation sub-domain of the Vineland-3 between 52 weeks of treatment and baseline was 6.5, 95% confidence interval=−2.131, 15.131, p value=0.1181.

One patient achieved scores within the normative mean range: Adaptive Behaviour Composite=102, percentile rank of 55; Communication=96, percentile rank of 39; Daily Living Skills=101, percentile rank of 53; Socialisation=109, percentile rank of 73.

Clinical Global Impression Scale-Severity of Illness (CGI-S). The mean difference of the Severity Illness domain of the CGI-S scale between 52 weeks of treatment and baseline was-1.1, 95% confidence interval=−2.080, −0.120, p value=0.03179.

Social Responsive Scale, 2^{nd} Edition (SRS-2). The mean difference of the total T-Score of the SRS-2 between 52 weeks of treatment and baseline was-4.1, 95% confidence interval=−8.170, −0.033, p value=0.04852. See Table 18 for further presentation of the results.

TABLE 18
SRS-2 Results

| | P-value | P-value |
|---|---|---|
| | (Paired T-Test) | (Paired T-Test) |
| SRS-2 Domain | 20 weeks | 52 weeks |
| Total | 0.012 | 0.049 |
| Social awareness | 0.596 | 0.421 |
| Social cognition | 0.028 | 0.105 |
| Social communication | 0.019 | 0.216 |
| Social motivation | 0.118 | 0.005 |
| Restricted interest and | 0.009 | 0.109 |
| repetitive behaviour | | |
| Social communication | 0.029 | 0.081 |
| and interaction | | |

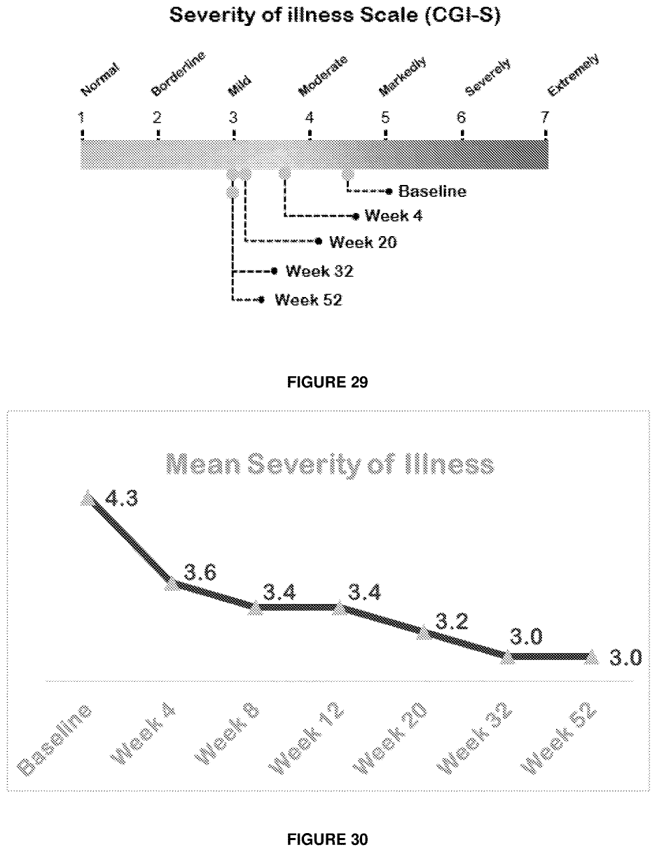

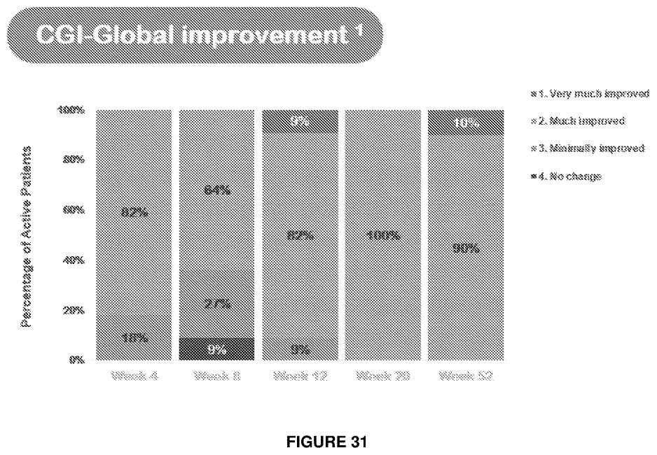

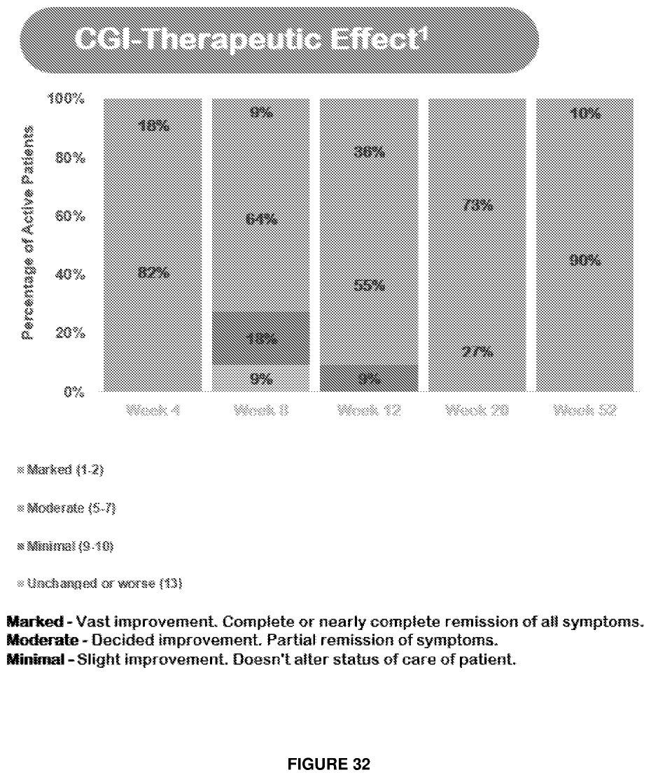

Please see FIGS. 29 to 32 for further presentation of the results.

### Safety Results.

The safety data concludes that NTI164 at 5, 10, 15 and 20 mg/kg administered in two doses daily up to 52-weeks, is safe and well-tolerated in this study population. A total of 6 adverse events were reported by four participants between week 20 and week 52. None of these adverse events were serious and were not considered to significantly interfere with the patient's functioning.

### Conclusion.

NTI164 was shown to be safe and well tolerated up to doses of 20/mg/kg/day. NTI164 has shown statistically significant efficacy in improving the symptoms associated with autism spectrum disorder after 52 weeks of daily therapy.

### Example 13-NTI164 for Treatment of Pans

### L.1 Background

The studies presented above using human microglial and neuronal cells have demonstrated that NTI164 has the ability to reduce microglial inflammation, suppress COX-2, suppress iNOS activation and “regulate” pro-inflammatory processes that allow cells to return back to “normal” status.

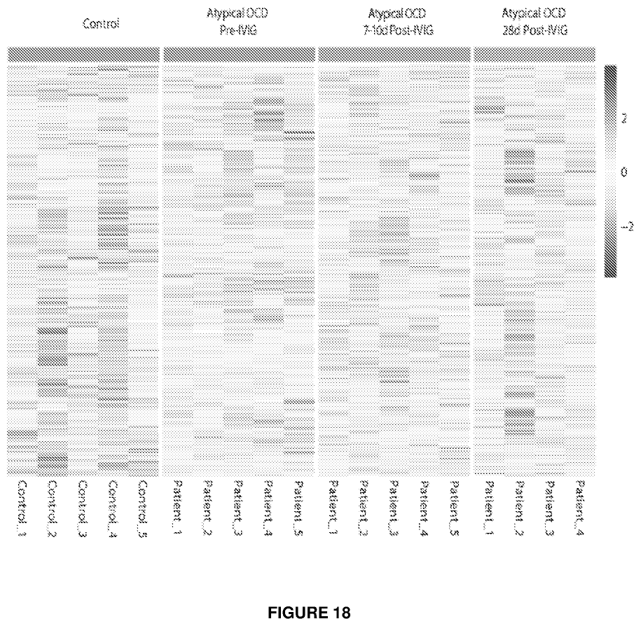

Despite ongoing debates about the clinical definition and etiology of paediatric acute-onset neuropsychiatric syndrome (PANS), experts agree that atypical, sudden-onset obsessive-compulsive disorder (OCD) associated with other change in function, exists as a phenomenon, and the prevalence may be increasing. Although many vulnerability genes have been identified for OCD, disease expression is driven by complex gene-environment interactions. Common environmental risk factors include perinatal stress and maternal inflammation during pregnancy (e.g., infections, autoimmune disease). Population studies and previous work have shown that many children with PANS or atypical OCD display evidence of immune dysfunction, such as recurrent infections and infection-triggered worsening of symptoms. When conventional psychiatric medications fail, immunomodulatory treatments, such as intravenous immunoglobulin (IVIG) and antibiotics have shown some therapeutic success in patients with atypical OCD. However, a lack of biomarkers and limited understanding of disease mechanisms has impeded further therapeutic discovery, including precision medicine approaches. A transcriptomic signature of immune dysfunction in children with atypical, sudden-onset OCD (PANS) using RNA sequencing of whole blood has been identified. PANS is an epigenetic immune-brain dysregulation syndrome, possibly related to microglial activation due to immune priming in pregnancy or early life. Differential gene expression and bioinformatic pathway-driven analysis has been used to identify common pathways of difference between patients and controls (FIG. 18).

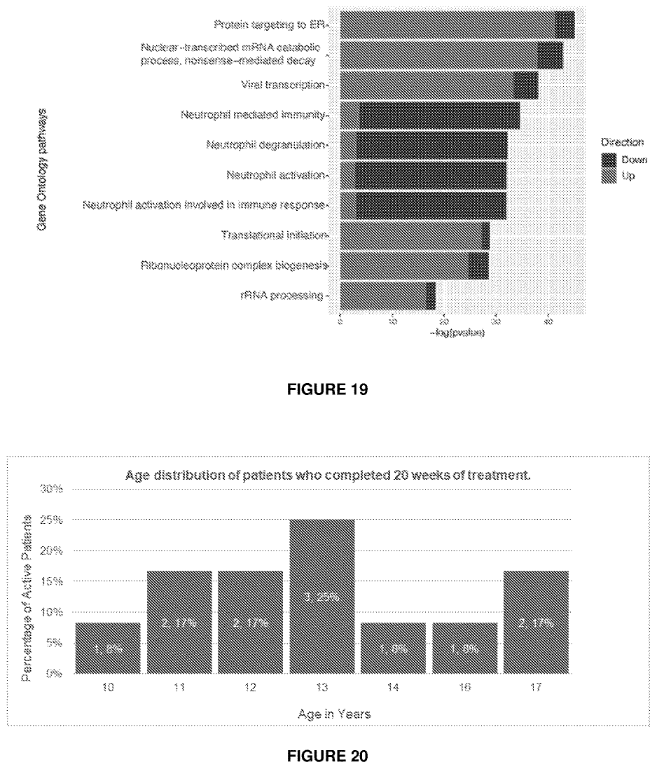

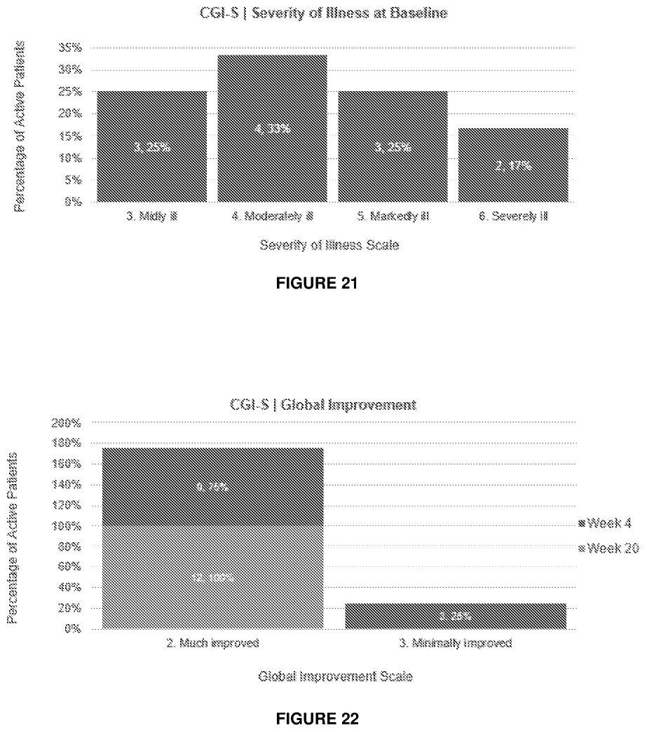

Using bioinformatic approaches, it has been shown that immune pathways and gene translation pathways are dysregulated in children with PANS compared to neurotypical controls (FIG. 19). Data shows that this disease signature is modifiable with IVIG treatment (FIG. 18) and associated with clinical improvements in these children.

IVIG is invasive, as it involves infusing immunoglobulin via a cannula every 4 weeks. The therapeutic benefits typically only lasts 2-3 weeks, with symptoms returning the week before another dose of IVIG is due. Therefore, alternative anti-inflammatory approaches are needed. NTI164 is a very attractive product for this purpose. NTI164 has clear anti-inflammatory effects, and low THC content, therefore it is appropriate for a paediatric application

### L.2 Study Aim

To examine the potential immunomodulatory, anti-inflammatory, behavioural and gene regulatory effects of NTI164 in children with PANS/PANDAS in an open-label setting, using our pre-post ‘omic’ approaches.

The study showed significant improvements in behavioural outcomes children with atypical, abrupt-onset OCD (PANS phenotype)

### L.3 Study Design and Methods

### Participants

### Patient Group:

A cohort of 15 children (aged 2-17 years) who fulfil PANS/PANDAS criteria were recruited from clinics.

Inclusion criteria of patient group: (1) Fulfil PANS criteria (E. Swedo S. From research subgroup to clinical syndrome: modifying the PANDAS criteria to describe PANS (pediatric acute-onset neuropsychiatric syndrome). Pediatrics & Therapeutics. 2012; 02 (02); Chang K, Frankovich J, Cooperstock M, et al. Clinical evaluation of youth with pediatric acute-onset neuropsychiatric syndrome (PANS): recommendations from the 2013 PANS Consensus Conference. J Child Adolesc Psychopharmacol. 2015; 25 (1): 3-13); and (2) Children's Yale-Brown Obsessive Compulsive Scale (CY-BOCS) score higher than 24 (moderate or higher) inferring significant impact. Patients can be on other medication (eg SSRI) but need to be established on this treatment for at least 3 months. Patients should not be on IVIG (can recruit if off IVIG for 3 months or longer).

### Research Control Group:

A research control group of 15 children provided a baseline comparison of transcriptomic signature and immune function only between participants with PANS/PANDAS and neurotypical children. Children without neurodevelopmental disorders who are undergoing planned blood investigations (e.g., endocrine investigation for short stature) were recruited. Hence, their research blood collection for this study can be aligned with a clinical collection to minimise patient discomfort. The research control group was recruited such that the sex ratio and age (mean, median, range) was not significantly different to the patient group, enabling data analysis of sex effects. Language interpreters were provided where necessary.

Inclusion criteria of neurotypical controls: (1) No definable immune, autoimmune, or inflammatory conditions; (2) No neurodevelopmental or neuropsychiatric disorders; and (3) Not receiving any immunomodulatory therapy.

### Treatment

An open-label clinical study of NTI164 in 15 children with PANS/PANDAS. The patient group were recruited under an ethically approved clinical trial process.

### The Study Protocol

The study involved the following phases:

Baseline: Patients were assessed and scoring as above completed, and baseline bloods taken as below.

Baseline/Up-titration Phase: Patients received a baseline dose of 5 mg/kg/day of NTI164 that was increased weekly by 5 mg/kg for a period of 4 weeks until the maximum tolerated dose or 20 mg/kg is achieved.

Treatment Phase: Patients received the maximum tolerated dose daily or 20 mg/kg/day for either an 8-week period.

Clinical Monitoring of Patient Group: All participants (n=15) were subject to routine clinical assessment using screening tools developed by our team. This assessment covers demographics, family medical history, maternal health during pregnancy, and child's medical history. Disease severity was assessed at baseline using the following gold-standard, validated clinical assessment tools that are part of standard care in neurology. The primary problem in this cohort is OCD, hence the CY-BOCS was the main outcome measure. However, these patients have broader clinical problems so gold standard measures for anxiety and depression tics, emotional control, and attention was also gathered: Revised Child Anxiety and Depression Scale-Parent Version (RCADS-P) (Becker, S. P., et al., Psychometric validation of the Revised Child Anxiety and Depression Scales-Parent Version (RCADS-P) in children evaluated for ADHD. Assessment, 2019. 26 (5): p. 811-82), Children's Yale-Brown Obsessive Compulsive Scale (CY-BOCS) (Scahill L, Riddle M A, McSwiggin-Hardin M, et al. Children's Yale-Brown Obsessive Compulsive Scale: reliability and validity. J Am Acad Child Adolesc Psychiatry. 1997; 36 (6): 844-852); Yale Global Tic Severity Scale (YGTSS) (Leckman J F, Riddle M A, Hardin M T, et al. The Yale Global Tic Severity Scale: initial testing of a clinician-rated scale of tic severity. J Am Acad Child Adolesc Psychiatry. 1989; 28 (4): 566-573); Clinical Global Impression (CGI) (Busner J, Targum S D. The clinical global impressions scale: applying a research tool in clinical practice. Psychiatry (Edgmont). 2007; 4 (7): 28-37); Children's Global Assessment Scale (CGAS) (Green B, Shirk S, Hanze D, Wanstrath J. The Children's Global Assessment Scale in clinical practice: an empirical evaluation. J Am Acad Child Adolesc Psychiatry. 1994; 33 (8): 1158-1164; Shaffer D, Gould M S, Brasic J, et al. A children's global assessment scale (CGAS). Arch Gen Psychiatry. 1983; 40 (11): 1228-1231); Strengths and Difficulties Questionnaire (SDQ) (Goodman R. Psychometric properties of the strengths and difficulties questionnaire. J Am Acad Child Adolesc Psychiatry. 2001; 40 (11): 1337-1345); Spence Children's Anxiety Scale (Spence)—The generalised anxiety disorder/overanxious disorder subscale (questions 1, 3, 4, 18, 20, 22) (Spence S H. A measure of anxiety symptoms among children. Behav Res Ther. 1998; 36 (5): 545-566); Conner's Abbreviated Rating Scale (Conner's) (Volpe R J, Gadow K D. Creating abbreviated rating scales to monitor classroom inattention-overactivity, aggression, and peer conflict: reliability, validity, and treatment sensitivity. School Psych Rev. 2019; 39 (3): 350-363); Affective Reactivity Index (ARI) (Stringaris A, Goodman R, Ferdinando S, et al. The Affective Reactivity Index: a concise irritability scale for clinical and research settings. J Child Psychol Psychiatry. 2012; 53 (11): 1109-1117).

Transcriptomics-RNA Sequencing: Methylomic and immune biomarker assessments was also carried out as part of a series of observational analysis.

### L.4 Study Results

### Summary of Results

TABLE 19
Summary of t-test scores for all endpoints

| | | | 95% | | |
|---|---|---|---|---|---|
| | | Mean | Confidence | | |
| Test | Subscale | Difference | Interval | | P value |
| RCADS-P | Total Score | −25.5 | 7.121 | 43.879 | 0.0161 |
| RCADS-P | Social Phobia | −6.2 | 3.560 | 8.773 | 0.00174 |
| RCADS-P | Panic Disorder | −2.8 | −1.172 | 6.839 | 0.1287 |
| RCADS-P | Major Depression | −2.7 | −1.461 | 6.794 | 0.1576 |
| RCADS-P | Separation Anxiety | −5.3 | 0.471 | 10.196 | 0.03712 |
| RCADS-P | Generalised Anxiety | −5.5 | 1.263 | 9.737 | 0.02062 |
| RCADS-P | Obsessive-Compulsive | −4.2 | 0.161 | 8.172 | 0.04413 |
| CGI-S | Severity of Illness | −0.9 | 0.680 | 1.197 | 0.0005371 |
| Yale Global Tic Severity Scale | Total Score | −7.3 | −22.421 | 2.421 | 0.07418 |
| Yale Global Tic Severity Scale | Motor Tic Severity | −3.1 | −7.461 | 0.128 | 0.05327 |
| Yale Global Tic Severity Scale | Vocal Tic Severity | +4.9 | −12.391 | 5.057 | 0.2123 |
| Yale Global Tic Severity Scale | Total Tic Severity | +1.8 | −12.821 | 15.821 | 0.7608 |
| CY-BOCS | Total Score | −3 | −24.461 | 23.794 | 0.958 |
| CY-BOCS | Obsession | −2.7 | −7.838 | 2.504 | 0.1567 |
| CY-BOCS | Compulsion | +0.6 | −17.591 | 22.258 | 0.6644 |
| Conner's | Total Score | −3.3 | −0.892 | 11.892 | 0.07793 |
| EQ-5D-Y | Total Health | +12.9 | −0.007 | 15.341 | 0.05014 |

### Primary Endpoints

Revised Children's Anxiety and Depression Scale, Parent-rated (RCADS-P)

The RCADS-P is a 47-item parent-reported questionnaire that measures symptoms of depression and anxiety in children and adolescents. The RCADS-P consists of six subscales helpful in screening children for high prevalence disorders, including: Separation anxiety disorder (SAD)
Social phobia (SP)
Generalised anxiety disorder (GAD)
Panic disorder (PD)
Obsessive compulsive disorder (OCD).
Major depressive disorder (MDD).

TABLE 20
Score ranges for each subscale of the RCADS-P

| Subscale | Range |
|---|---|
| Total Score | 0-141 |
| Social Phobia | 0-27 |
| Panic Disorder | 0-27 |
| Major Depression | 0-30 |
| Separation Anxiety | 0-18 |
| Generalised Anxiety | 0-18 |
| Obsessive-Compulsive | 0-18 |

The Paired t-test was used to assess statistical significance of the analysed data sets. The mean difference of the Total RCADS-P score between baseline and 12 weeks of treatment was-25.5, 95% confidence interval=7.121, 43.879, p value=0.0161 (see Table 19).

TABLE 21
Grouped means of the Total scores and Subscale scores of the Revised
Children's Anxiety and Depression Scale, Parent-rated (RCADS-P)
at week 12 (where a decrease in score demonstrates improvement).

| | Baseline | Week 12 | Max | Max | Mean |
|---|---|---|---|---|---|
| RCADS-P | Mean | Mean | decrease | increase | Difference |
| Total Score | 83.7 | 58.2 | −49 | Nil | −25.5 |
| Social Phobia | 19.2 | 13.0 | −10 | Nil | −6.2 |
| Panic Disorder | 10.2 | 7.3 | −10 | Nil | −2.8 |
| Major Depression | 17.3 | 14.7 | −7 | +3 | −2.7 |
| | | | | (n = 1) | |
| Separation Anxiety | 14.8 | 9.5 | −13 | Nil | −5.3 |
| Generalised Anxiety | 13.2 | 7.7 | −12 | Nil | −5.5 |
| Max score 18 | | | | | |
| Obsessive | 9.0 | 4.8 | −10 | Nil | −4.2 |
| Compulsive | | | | | |
| Max score 18 | | | | | |

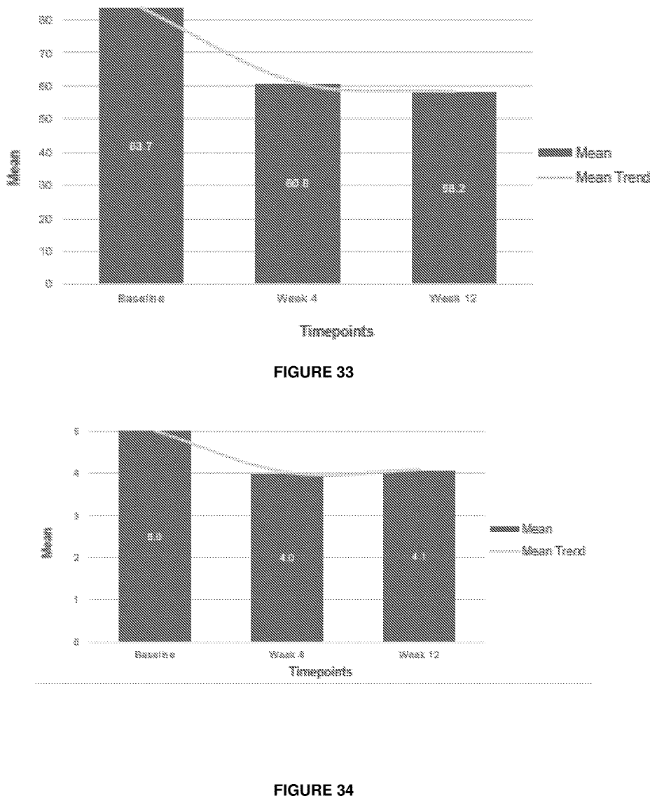

### See Tables 19, 20 and 21, and FIG. 33.

### Clinical Global Impression-Severity (CGI-S)

The Clinical Global Impression-Severity (CGI-S) scale was used to assess: (a) Severity of Illness: a comparison of baseline and week 12 of NTI164 treatment; and (b) Therapeutic Effect: rated based on drug effect only. This is a calculated score based on the degrees of therapeutic effect and side effects.

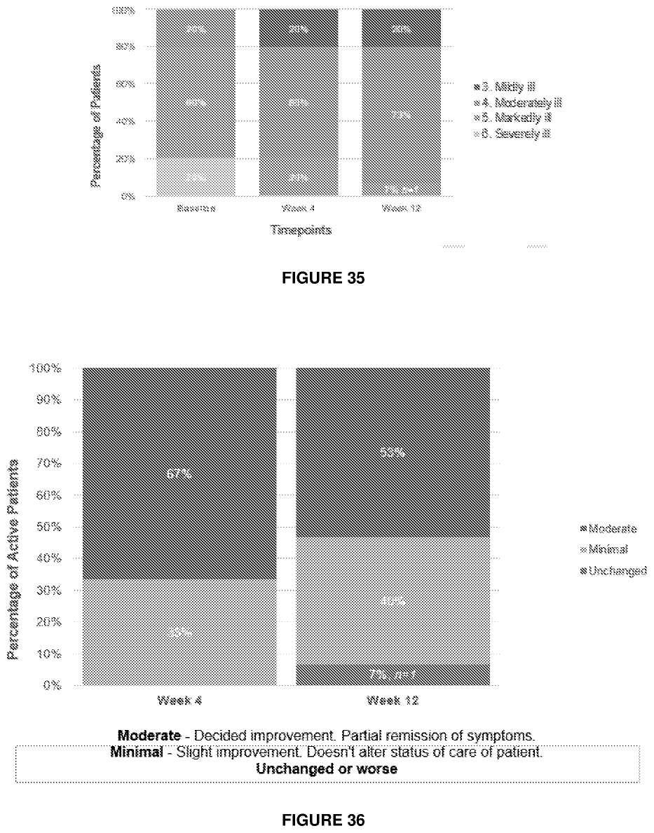

Severity of Illness. The average rating for the severity of illness at baseline was 5. This reduced to an average rating of 4.1 after 12 weeks of daily NTI164 treatment. See FIG. 34. The mean difference of the severity of illness scores between baseline and 12 weeks of treatment was-0.9, 95% confidence interval=0.680, 1.197, p value=0.0005371 (see Table 19). See FIG. 35.

Therapeutic Effect. After 12 weeks of daily NTI164 treatment, 53% of active patients demonstrated a Moderate improvement and 40% demonstrated a Minimal improvement, only one patient (7%) was rated as being Unchanged. See FIG. 36.

### Clinical Global Impression-Improvement (CGI-I)

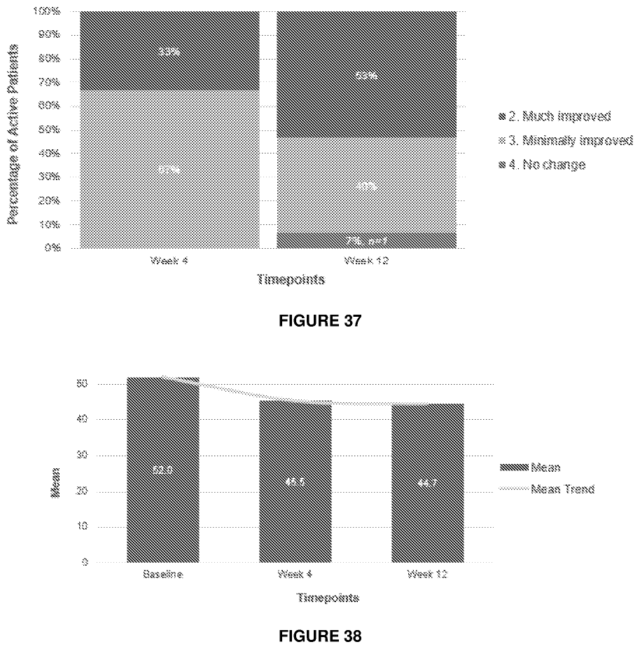

The Clinical Global Impression-Improvement (CGI-I) scale was used to assess the overall improvement of the patient where 53% of patients experienced Much improvement, 40% experienced Minimal improvement and only one patient experienced No change (7%). See FIG. 37.

### Secondary Endpoints

### Yale Global Tic Severity Scale (YGTSS)

The YGTSS is a tool used to quantify the severity of Tic symptoms in individuals aged 6-17. The YGTSS is made up of a semi-structured interview, followed by a questionnaire where individuals are asked to rate the severity of their Tic symptoms (both motor and vocal) in domains such as: number, frequency, intensity, complexity, and interference.

The Total Tic Severity Score has a range of 0-50, and the Global Tic Severity Score has a range of 0-100.

A higher score on all scales suggests a more severe Tic, or a greater impact the Tic has on the person's life

See Table 22.

TABLE 22
Grouped means of the Total scores and Subscale scores of the Yale Global Tic Severity
Scale (YGTSS) at week 12 (where a decrease in score demonstrates improvement).

| | Baseline | Week 12 | Max | Max | Mean |
|---|---|---|---|---|---|
| YGTSS | Mean | Mean | decrease | Increase | Difference |
| Total Yale Global Tic Severity Scale Score | 52.0 | 44.7 | −15 | Nil | −7.3 |
| {Total Tic Seventy Score + Impairment) (0-100) | | | | | |
| Total Motor Tic Severity | 15.8 | 12.7 | −5 | Nil | −3.1 |
| Total Vocal Tic Severity | 3.8 | 8.7 | 0 | +7 (n = 2) | +4.9 |
| Total Tic Severity Score | 19.5 | 21.3 | −5 | +5 (n = 1) | +1.8 |
| (Motor Tic Severity + Vocal Tic Severity) (0-50) | | | | | |

See FIG. 38.

### Children's Yale-Brown Obsessive-Compulsive Scale (CY-BOCS)

The CY-BOCS is a scale is designed to rate the severity of obsessive and compulsive symptoms in children and adolescents, ages 6-17 years and is administered by the Clinician.
“Obsessions” are considered as thoughts, ideas, or pictures that keep coming into mind even though you do not want them to. They may be unpleasant, silly, or embarrassing.
“Compulsions” are things that you feel you have to do although you may know that they do not make sense. Sometimes you may try to stop from doing them but this might not be possible. You might feel worried or angry or frustrated until you have finished what you have to do

### See Table 23.

TABLE 23
Total CY-BOCS score severity ranges.

| Severity | Total CY-BOCS Score |
|---|---|
| Subclinical | 0-7 |
| Mild | 8-15 |
| Moderate | 16-23 |
| Severe | 24-31 |
| Extreme | 32-40 |

TABLE 24
Grouped means of the Total scores and Subscale scores of the Children's
Yale-Brown Obsessive-Compulsive Scale (CY-BOCS) at week 12
(where a decrease in score demonstrates improvement)

| | Baseline | Week 12 | Max | Max | Mean |
|---|---|---|---|---|---|
| CY-BOCS | Mean | Mean | decrease | increase | Difference |
| CY-BOCS | 33.0 | 30.0 | −11 | +8 (n = 2) | −3.0 |
| Total Score | | | | | |
| Obsession subtotal | 17.3 | 13.7 | −5 | Nil | −3.6 |
| Compulsion subtotal | 15.8 | 16.3 | −6 | +10 (n = 2) | +0.6 |

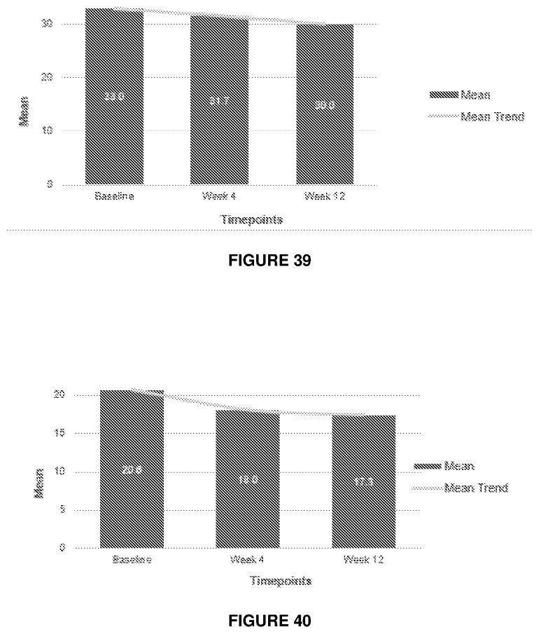

See FIG. 39.

### Conner's Scale (Assessment of Attention Deficit Hyperactivity Disorder (ADHD) Behaviours).

The Conner's scale is an assessment tool used to obtain observations about a youth's behaviour. The instrument is designed to assess Attention Deficit/Hyperactivity Order (ADHD) and its most common co-morbid problems in children and adolescents aged 6 to 18 years old. A Total Score of 15 and above may be suggestive of ADHD.

See Table 25.

TABLE 25
Grouped mean of the Total score of the Conner's scale at week 12
(where a decrease in score demonstrates improvement).

| | Baseline | Week 12 | Max | Max | Mean |
|---|---|---|---|---|---|
| Conner's | Mean | Mean | decrease | increase | Difference |
| Total Score | 20.6 | 17.3 | −10 | +3 (n = 1) | −3.3 |

See FIG. 40.

### EQ-5D-Y (Standardised Measure of Health-Related Quality of Life Domains)

The EQ-5D-Y records the patient's self-rated health on a vertical visual analogue scale (VAS) where the endpoints are labelled “The best health you can imagine” (equates to a score of 100) and “The worst health you can imagine” (equates to a score of 0).

See Table 26.

TABLE 26
Grouped means of the Total score of the EQ-5D-Y scale at week 12
(where a decrease in score demonstrates improvement).

| | Baseline | Week 12 | Max | Max | Mean |
|---|---|---|---|---|---|
| EO-5D-Y | Mean | Mean | decrease | increase | Difference |
| Health Score | 70.6 | 83.5 | −10 (n = 0) | +20 | +12.9 |

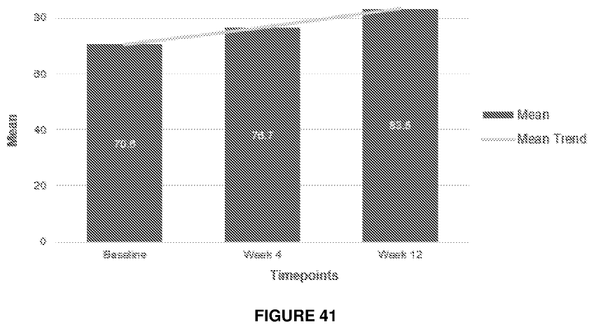

See FIG. 41.

### L.5 Discussion of Results

The results demonstrate that children with atypical, abrupt-onset OCD (PANS/PANDAS phenotype) have clinical improvements in neuropsychiatric symptoms (using CGI, CY-BOCS and RCADS-P as primary endpoints; Becker, S. P., et al., Psychometric validation of the Revised Child Anxiety and Depression Scales-Parent Version (RCADS-P) in children evaluated for ADHD. Assessment, 2019. 26 (5): p. 811-824). “Omic” and immune biomarker studies show changes consistent with clinical improvements.

Statistically significant p-values were observed when assessing the study's primary endpoint, the R-CADS-P scale, namely within the Total score (p=0.0161) and subscales: Social Phobia (p=0.00174), Separation Anxiety (0.03712), Generalised Anxiety (0.02062) and the Obsessive-Compulsiveness (p=0.04413).

For the CGI-S, the mean difference in the Severity of Illness subscale was-0.9 after 12 weeks of treatment and with 53% of patients experiencing a Moderate improvement based on therapeutic effect, 40% experiencing Minimal improvement and only one patient (7%) being rated as Unchanged.

For the CGI-I, all patients demonstrated improvement at the 4-week timepoint (33% Much improved, 67% Minimally improved); after 12 weeks of treatment, 53% of patients demonstrated Much improvement, 33% demonstrated Minimal improvement and only one patient (13%) was rated as having No change.

These results demonstrate that the daily administration of NTI164 in treating paediatric acute-onset neuropsychiatric syndrome is efficacious.

### M Example 14-NTI164 for Treatment of Rett Syndrome

### M.1 Background

Rett syndrome (RTT) is a pervasive neurological disorder. This genetic neurological disorder primarily affects females, and very rarely can be found in males. RTT is a neurodegenerative disease, characterised by a seemingly normal prenatal and perinatal period and healthy development for 6-18 months, and followed by a period of neurodevelopmental stagnation, and then rapid regression. During regression, infants will lose motor, cognitive and communication skills. In most cases, Rett syndrome is caused by a mutation within the methylcytosine-binding protein 2 (MECP2) gene.

### M.2 Study Aim

To examine the potential immunomodulatory, anti-inflammatory, behavioural and gene regulatory effects of NTI164 in female children with Rett Syndrome.

The open label study will show that: significant improvements in behavioural outcomes in female children with Rett Syndrome; NTI164 will assist with communication, attentiveness, and eye contact; and functional impairment resulting from Rett symptoms will be reduced with NTI164 treatment.

### M.3 Participants

### Patient Group:

A cohort of 14 female children (aged 5-20 years) with classical/typical RTT will be recruited from clinics.

Inclusion criteria of patient group will include: Girls and women, aged 5-20 years; Weight ≥12 kg; Classical/typical RTT; Documented disease-causing mutation in MECP2 gene; At least 6 months post regression at screening (ie. no loss or degradation in ambulation, hand function, speech, nonverbal communicative or social skills within 6 months of screening); Rett Syndrome Clinical Severity Scale rating of 10-36; CGI score of ≥4; and Stable pattern of seizures, or has had no seizures, within 8 weeks of screening.

Exclusion criteria of patient group will include: Current clinically significant cardiovascular, endocrine (such as hypo- or hyperthyroidism, type 1 diabetes, or uncontrolled type 2 diabetes), renal, hepatic, respiratory, or gastrointestinal disease (such as celiac disease or inflammatory bowel disease), or major surgery planned during the study; Known history or symptoms of long QT syndrome; QTcF interval >450 ms, history of risk factor for torsades de pointes or clinically significant QT prolongation deemed to increase risk; Treatment with insulin, IGF-1, or growth hormone within 12 weeks of baseline; Currently receiving treatment with DAYBUE™ (trofinetide); Currently using other unregistered drugs for the treatment of Rett syndrome such as Anavex; Currently using or has used recreational or medicinal Cannabis, cannabinoid-based medications (including Sativex®, or Epidiolex®) within the 12 weeks prior to screening and is unwilling to abstain for the duration of the trial; and Participant has any known or suspected hypersensitivity to cannabinoids or any of the excipients.

### Research Control Group:

A research control group of 14 female children will provide a baseline comparison of transcriptomic signature and immune function only between participants with classical/typical RTT and neurotypical children. Children without neurodevelopmental disorders who are undergoing planned blood investigations (e.g., endocrine investigation for short stature) will be recruited. Hence, their research blood collection for this study can be aligned with a clinical collection to minimise patient discomfort. The research control group will be recruited such that the sex ratio and age (mean, median, range) is not significantly different to the patient group, enabling data analysis of sex effects. Language interpreters will be provided where necessary. Inclusion criteria of neurotypical controls: (1) No definable immune, autoimmune, or inflammatory conditions; (2) No neurodevelopmental or neuropsychiatric disorders; and (3) Not receiving any immunomodulatory therapy.

### M.4 Treatment

An open-label clinical study of NTI164 in 14 female children with classical/typical RTT. The patient group will be recruited under an ethically approved clinical trial process.

### M.5 Study Protocol

The study involves the following phases:

Baseline: Patients will be assessed and scoring as above will be completed, and baseline bloods will be taken as below.

Baseline/Up-titration Phase: Patients will receive a baseline dose of 5 mg/kg/day of NTI164 that will be increased weekly by 5 mg/kg for a period of 4 weeks until the maximum tolerated dose or 20 mg/kg is achieved.

Treatment Phase: Patients will receive the maximum tolerated dose daily or 20 mg/kg/day for either an 8-week period.

Clinical Monitoring of Patient Group: All participants (n=14) will undergo a routine clinical assessment using screening tools developed by our team. This assessment covers demographics, family medical history, maternal health during pregnancy, and child's medical history. Disease severity will be assessed at baseline using the following gold-standard, validated clinical assessment tools that are part of standard care in neurology.

The Clinical data questionnaire will include:

Clinical Global Impression Scale-Improvement (CGI-I), This is a 7-point scale measuring symptom change from baseline. It will be provided as baseline and post-baseline Caregiver and Clinician questionnaires. [Timeframe: Baseline, Weeks 5, 13 & 15].

Rett Syndrome: Symptom Index Score (RTT-SIS). A questionnaire developed by A/Prof Ellaway which monitors difference and changes in range of symptoms associated with RTT. [Timeframe: Baseline, Week 13].

Clinical Global Impression Scale-Severity (CGI-S). Reflects clinician's impression of severity of illness on a 7-point scale ranging from 1=not at all to 7=among the most extremely ill. [Timeframe: Baseline, Weeks 5, 13 & 15].

Rett Syndrome Behaviour Questionnaire (RSBQ). The RSBQ is a 45-item rating scale completed by the caregiver that assesses a range of neurobehavioral features impaired in RTT. Symptoms assessed include mood disruption, maladaptive behaviours, fear/anxiety, repetitive movements, hand behaviours, breathing abnormalities, and gross motor skills (walking/standing). The caregiver rates items as 0=not true, 1=somewhat or sometimes true, or 2=very true. This instrument is validated and FDA-accepted for RTT studies and used widely to assess RTT symptoms in a number of RTT studies. Floor score is 4 (minimum score/worst), ceiling score (maximum score/best) is 75. [Timeframe: Baseline, Weeks 5, 13 & 15].

RTT-Clinician Domain Specific Concerns-Visual Analog Scale (RTT-DSC-VAS). Each area of functional impairment is rated on an 8-point Likert scale, from 0=normal function to 7=most severe impairment: hand function, ambulation and gross motor skills, the ability to communicate choices or preferences (including the use of nonverbal means such as eye contact and gestures), and the ability to communicate verbally. For each rating, function during the previous visit is assessed. The rater may rely on both the report of caregivers and the rater's own observation and examination. [Timeframe: Baseline, Weeks 5, 13 & 15].

Communication and Symbolic Behaviour Scales Developmental Profile™ Infant-Toddler Checklist (CSBS-DP-IT Social). The checklist consists of 24 items completed by the caregiver. Each item is scored using a three-level rating of frequency: “not yet”, “sometimes”, and “often.” The checklist is composed of three composite scores: Social, Speech, and Symbolic. The first 13 items make up the Social composite score, and include Emotion and Eye Gaze, Communication, and Gestures components. The items comprising the Social composite score are the modalities most commonly used by people with RTT to communicate [41,42]. The Social composite score has been used previously in a placebo-controlled study of mecasermin in the treatment of RTT [20]. Identification by the CSBS-DP-IT of a delay in skills suggests permanent rather than temporary delay.′ [Timeframe: Baseline, Weeks 5, 13 & 15].

Impact of Childhood Neurologic Disability Scale (ICND). The total score of the ICND evaluates the impact that a child's condition has on the child's and the family's everyday life at the present time and during the previous 3 months. The parent or caregiver evaluates the effect of four conditions or health problems (inattentiveness, impulsivity, or mood; ability to think and remember; neurologic or physical limitations; epilepsy) on 11 aspects of the child's or the family's life as: “A lot”, “Some”, “A little”, “Not at all”, or “Does not apply.” [Timeframe: Baseline, Week 13].

RTT Caregiver Burden Inventory (RTT-CBI). This is a syndrome-specific, caregiver-completed questionnaire that is based on the Caregiver Burden Inventory designed for Alzheimer's disease. The RTT-CBI assesses four different aspects of burden (physical, emotional, and social burden, and time dependence). Caregivers use a 5-point Likert scale to rate the frequency that each statement describes their feeling or experience as: 0=never; 1=rarely; 2=sometimes; 3=frequently, and 4=nearly always. Items 1-24 are negatively worded, with a total maximal score of 96. The total score is defined as the Total Burden Score. [Timeframe: Baseline, Week 13].

Overall Quality of Life Rating of the Impact of Childhood Neurologic Disability Scale (ICND-QOL). The quality of life of the individual is assessed by asking the caregiver to rate the subject's overall quality of life on a 6-point scale from 1 (“Poor”) to 6 (“Excellent”). [Timeframe: Baseline, Week 13].

Blood tests will be performed at each hospital visit and will include routine monitoring/safety tests including checking full blood count (FBC), electrolytes, creatinine, T and B cell subsets, immunoglobulin G/A/M levels, and liver and kidney function tests.

The primary problem in this cohort is OCD, hence the CY-BOCS will be the main outcome measure. However, these patients have broader clinical problems so gold standard measures for tics, emotional control, and attention will also be gathered:
Children's Yale-Brown Obsessive Compulsive Scale (CY-BOCS) (Scahill L, Riddle M A, McSwiggin-Hardin M, et al. Children's Yale-Brown Obsessive Compulsive Scale: reliability and validity. J Am Acad Child Adolesc Psychiatry. 1997; 36 (6): 844-852);
Yale Global Tic Severity Scale (YGTSS) (Leckman J F, Riddle M A, Hardin M T, et al. The Yale Global Tic Severity Scale: initial testing of a clinician-rated scale of tic severity. J Am Acad Child Adolesc Psychiatry. 1989; 28 (4): 566-573); Clinical Global Impression (CGI) (Busner J, Targum S D. The clinical global impressions scale: applying a research tool in clinical practice. Psychiatry (Edgmont). 2007; 4 (7): 28-37); Children's Global Assessment Scale (CGAS) (Green B, Shirk S, Hanze D, Wanstrath J. The Children's Global Assessment Scale in clinical practice: an empirical evaluation. J Am Acad Child Adolesc Psychiatry. 1994; 33 (8): 1158-1164; Shaffer D, Gould M S, Brasic J, et al. A children's global assessment scale (CGAS). Arch Gen Psychiatry. 1983; 40 (11): 1228-1231); Strengths and Difficulties Questionnaire (SDQ) (Goodman R. Psychometric properties of the strengths and difficulties questionnaire. J Am Acad Child Adolesc Psychiatry. 2001; 40 (11): 1337-1345); Spence Children's Anxiety Scale (Spence)—The generalised anxiety disorder/overanxious disorder subscale (questions 1, 3, 4, 18, 20, 22) (Spence S H. A measure of anxiety symptoms among children. Behav Res Ther. 1998; 36 (5): 545-566); Conner's Abbreviated Rating Scale (Conner's) (Volpe R J, Gadow K D. Creating abbreviated rating scales to monitor classroom inattention-overactivity, aggression, and peer conflict: reliability, validity, and treatment sensitivity. School Psych Rev. 2019; 39 (3): 350-363); Affective Reactivity Index (ARI) (Stringaris A, Goodman R, Ferdinando S, et al. The Affective Reactivity Index: a concise irritability scale for clinical and research settings. J Child Psychol Psychiatry. 2012; 53 (11): 1109-1117).

Transcriptomics-RNA Sequencing: Methylomic and immune biomarker assessments will be carried out as part of a series of observational analysis.

### M.6 Study Results

The results will demonstrate that female children with classical/typical RTT will demonstrate:
(1) The average CGI-I score is 3 or less at the end of the study treatment;
(2) An improvement in the clinically used, gold-standard measures of assessing symptoms, behaviour, concerns, development, disability and quality of life will be our secondary outcome measures, including: Rett Syndrome: Symptom Index Score (RTT-SIS); Rett Syndrome Behaviour Questionnaire (RSBQ); RTT-Clinician Domain Specific Concerns Visual Analog Scale (RTT-DSC-VAS); Communication and Symbolic Behaviour Scales Developmental Profile™ Infant-Toddler Checklist (CSBS-DP-IT Social); Impact of Childhood Neurological Disability Scale (ICND); RTT Caregiver Burden Inventory (RTT-CBI); Overall Quality of Life Rating of the Impact of Childhood Neurological Disability Scale (ICND-QOL);
(3) An improvement in the three domains of the Clinical Global Impression Scale Severity (CGI-S), Severity of Illness, Global Improvement and Therapeutic Effect; and
(4) That NTI164 was effective and safe in this paediatric population.

1. A method of treating a paediatric neurological disorder, said method comprising administering to a patient in need thereof a therapeutically effective amount of a composition, wherein the composition comprises the following cannabinoids:
w/w %
CBDA 40-60%;
CBD 1-5%;
CBG 1-10%;
CBDP 1-5%;
CBDB 1-5%;
CBGA 1-10%;
CBN 1-3%
and
THC<1%.

2. The method of claim 1 wherein, the cannabinoids of the composition are present in amounts selected from the group consisting of:
Composition 1 comprising
w/w %
CBDA 50%;
CBD 2%;
CBG 5%;
CBDP 2%;
CBDB 2%;
CBGA 5%;
CBN 3% and
THC<0.3%;
and
Composition 2 comprising
w/w %
CBDA 45%;
CBD 1%;
CBG 4%;
CBDP 1%;
CBDB 2%;
CBGA 4%;
CBN 2% and
THC<0.2%.

3. The method of claim 1 or 2, wherein the paediatric neurological disorder is selected from the group consisting of: PANS/PANDAS, Tourette syndrome, cerebral palsy, ataxia, attention deficit hyperactivity disorder (ADHD), Lennox Gastaut, Dravet syndrome, leukodystrophy and subtypes, Reye syndrome, Rett syndrome, Fragile-X syndrome, Phelan-McDermid syndrome, Angelman syndrome, Pitt-Hopkins syndrome, Prader-Willi syndrome, a metabolic disorder and a glycosylation disorder.

4. The method of claim 3, wherein the PANS is selected from the group consisting of: PANDAS and PITANDS.

5. The method of any one of the above-mentioned claims, wherein the composition further comprises an oil selected from the group consisting of: a synthetic oil; plant based oil; mineral oil; canola oil; and olive oil.

6. The method of any one of the above-mentioned claims, wherein the composition comprises less than 5% w/w terpenes.

7. The method of any one of the above-mentioned claims, wherein the composition comprises less than 2% w/w organic plant material.

8. The method of any one of the above-mentioned claims, wherein the composition comprises less than 2% w/w of plant phenols.

9. The method of any one of the above-mentioned claims, wherein the cannabinoid component of the composition is selected from the group consisting of: between 1 and 500 mg/ml; between 10 and 100 mg/ml; be at a concentration of 50 mg/ml.

10. The method of any one of the above-mentioned claims, wherein the CBDA component of the composition is selected from the group consisting of: between 1 and 500 mg/ml; between 10 and 100 mg/ml; be at a concentration of 50 mg/ml.

11. The method of any one of the above-mentioned claims, wherein the composition has a UPLC mass chromatogram corresponding to FIG. 3 utilising the conditions described in Example 1.

12. The method of any one of the above-mentioned claims, wherein the composition further comprises an additional active ingredient.

13. The method of claim 12, wherein additional active ingredient is selected from the group consisting of: diclofenac, prednisone, celecoxib and psylocibin.

14. A pharmaceutical composition adapted for treating a paediatric neurological disorder, said composition comprising the following cannabinoids:
w/w %
CBDA 40-60%;
CBD 1-5%;
CBG 1-10%;
CBDP 1-5%;
CBDB 1-5%;
CBGA 1-10%;
CBN 1-3%
THC<1%; and
together with a pharmaceutically acceptable carrier.

15. A dosage form adapted for treating a paediatric neurological disorder, said dosage form comprising the following composition:
w/w %
CBDA 40-60%;
CBD 1-5%;
CBG 1-10%;
CBDP 1-5%;
CBDB 1-5%;
CBGA 1-10%;
CBN 1-3%
THC<1%; and
wherein the CBDA component of the composition is selected from the group consisting of: between 1 mg and 1000 mg; between 1 mg and 500 mg; between 1 and 100 mg; less than 400 mg; less than 300 mg; less than 200 mg and less than 100 mg.

16. A dosage form adapted for treating a paediatric neurological disorder, said dosage form comprising the following composition:
w/w %
CBDA 40-60%;
CBD 1-5%;
CBG 1-10%;
CBDP 1-5%;
CBDB 1-5%;
CBGA 1-10%;
CBN 1-3%
THC<1%; and
wherein the CBDA component of the composition is selected from the group consisting of: 600 mg; 400 mg; 300 mg; 200 mg; 100 mg; 50 mg; 10 mg; 5 mg; 2 mg; 1 mg.

17. A kit adapted for treating a paediatric neurological disorder, said kit comprising the following composition:
w/w %
CBDA 40-60%;
CBD 1-5%;
CBG 1-10%;
CBDP 1-5%;
CBDB 1-5%;
CBGA 1-10%;
CBN 1-3%
THC<1%; and
together with instructions for use.

18. Use of a composition comprising the following cannabinoids:
w/w %
CBDA 40-60%;
CBD 1-5%;
CBG 1-10%;
CBDP 1-5%;
CBDB 1-5%;
CBGA 1-10%;
CBN 1-3%
and
THC<1%;
in the manufacture of a medicament for the treatment of a paediatric neurological disorder.

19. The use claim 18 wherein the cannabinoids of the composition are present in amounts selected from the group consisting of:
Composition 1 comprising
w/w %
CBDA 50%;
CBD 2%;
CBG 5%;
CBDP 2%;
CBDB 2%;
CBGA 5%;
CBN 3% and
THC<0.3%;
and
Composition 2 comprising
w/w %
CBDA 45%;
CBD 1%;
CBG 4%;
CBDP 1%;
CBDB 2%;
CBGA 4%;
CBN 2% and
THC<0.2%.

20. The use of claim 18 or 19, wherein the paediatric neurological disorder is selected from the group consisting of: PANS/PANDAS, Tourette syndrome, cerebral palsy, ataxia, attention deficit hyperactivity disorder (ADHD), Lennox Gastaut, Dravet syndrome, leukodystrophy and subtypes, Reye syndrome, Rett syndrome, Fragile-X syndrome, Phelan-McDermid syndrome, Angelman syndrome, Pitt-Hopkins syndrome, Prader-Willi syndrome, a metabolic disorder and a glycosylation disorder.

21. The use of claim 20, wherein the PANS is selected from the group consisting of: PANDAS and PITAND.

22. The use of any one of claims 18 to 21, wherein the composition further comprises an oil selected from the group consisting of: a synthetic oil; plant based oil; mineral oil; canola oil; and olive oil.

23. The use of any one of claims 18 to 22, wherein the composition comprises less than 5% w/w terpenes.

24. The use of any one of claims 18 to 22, wherein the composition comprises less than 2% w/w organic plant material.

25. The use of any one of claims 18 to 22, wherein the composition comprises less than 2% w/w of plant phenols.

26. The use of any one of claims 18 to 25, wherein the cannabinoid component of the composition is selected from the group consisting of: between 1 and 500 mg/ml; between 10 and 100 mg/ml; be at a concentration of 50 mg/ml.

27. The use of any one of claims 18 to 26, wherein the CBDA component of the composition is selected from the group consisting of: between 1 and 500 mg/ml; between 10 and 100 mg/ml; be at a concentration of 50 mg/ml.

28. The use of any one of claims 18 to 27, wherein the composition has a UPLC mass chromatogram corresponding to FIG. 3 utilising the conditions described in Example 1.

29. The use of any one of claims 18 to 28, wherein the composition further comprises an additional active ingredient.

30. The use of any claim 29, wherein the additional active ingredient is selected from the group consisting of: diclofenac, prednisone, celecoxib and psylocibin.

31. The methods, compositions, dosage forms, kits and uses as described by the foregoing examples.
